(19) United States
(12) Patent Application Publication
(10) Pub. No.: US 2005/0165025 A1
(43) Pub. Date: Jul. 28, 2005

(54) Combination therapy with 5HT 1A and 5HT 1B-receptor antagonists

(75) Inventors: Amedeo Leonardi, Milan (IT); Luciano Guarneri, Garbagnate Milanese (IT); Rodolfo Testa, Vignate (IT)

Correspondence Address: DARBY & DARBY P.C., P. O. BOX 5257, NEW YORK, NY, 10150-5257, US

(73) Assignee: Recordati Ireland Ltd., Ringaskiddy (IE)

(21) Appl. No.: 11/041,086

(22) Filed: Jan. 21, 2005

Related U.S. Application Data
(60) Provisional application No. 60/538,738, filed on Jan. 22, 2004.

(51) Int. Cl. A61K 31/496; A61K 31/495

(52) U.S. Cl. 514/252.12; 514/253.01; 514/254.06; 514/254.11; 514/253.06

(57) ABSTRACT

Described is the novel use of combinations of molecules endowed with antagonistic activity toward the serotonin 5-HT_{1A} or 5-HT_{1B} receptor, and of molecules simultaneously endowed with antagonistic activity at both said receptors. These compounds and their enantiomers, diastereoisomers, N-oxides, polymorphs, solvates, prodrugs, and pharmaceutically acceptable salts are useful in the treatment of patients with neuromuscular dysfunction of the lower urinary tract. Also described are the pharmaceutical compositions containing them. There is also provided a method of therapeutic treatment of urinary disorders in a mammal, including man, comprising administering to said mammal, including man, in need of such treatment, a therapeutically effective amount of a composition according to the invention.

### CROSS REFERENCE TO RELATED APPLICATION

The present application claims benefit of priority under 35 U.S.C. § 119(e) of provisional application No. 60/538,738, filed Jan. 22, 2004. The foregoing application is hereby incorporated by reference herein in its entirety.

### FIELD OF THE INVENTION

The invention is directed to treatment of disease of the lower urinary tract using a combination of HT_{1A} and HT_{1B} serotonin receptor antagonists.

### BACKGROUND OF THE INVENTION

In mammals, micturition (urination) is a complex process that requires the integrated action of the bladder, its internal and external sphincters, the musculature of the pelvic floor and neurological control over these muscles at three levels (in the bladder wall or sphincter itself, in the autonomic centers of the spinal cord and in the central nervous system at the level of the pontine micturition centre (PMC) in the brainstem (pons) under the control of the cerebral cortex) (De Groat, Neurobiology of Incontinence, Ciba Foundation Symposium 151:27, 1990). Micturition results from contraction of the detrusor muscle, which consists of interlacing smooth-muscle fibres, under the control of the parasympathetic autonomic system originating from the sacral spinal cord. A simple voiding reflex is triggered by sensory nerves for pain, temperature and distension that run from the bladder to the sacral spinal cord. However, sensory tracts from the bladder reach the PMC too, generating nerve impulses that normally suppress the sacral spinal suppression of cortical inhibition of the reflex arc, and relaxing the muscles of the pelvic floor and external sphincter. Finally, the detrusor muscle contracts and voiding occurs. Abnormalities of lower-urinary tract function, e.g., dysuria, incontinence and enuresis, are common in the general population. Dysuria includes urinary frequency, nocturia and urgency, and may be caused by cystitis (including interstitial cystitis), prostatitis or benign prostatic hyperplasia (BPH) (which affects about 70% of elderly males), or by neurological disorders. Incontinence syndromes include stress incontinence, urgency incontinence, overflow incontinence and mixed incontinence. Enuresis refers to the involuntary passage of urine at night or during sleep.

Previously, treatment of neuromuscular dysfunction of the lower urinary tract involved administration of compounds that act directly on the bladder muscles, such as flavoxate, a spasmolytic drug (Ruffman, J. Int. Med. Res. 16: 317, 1988) which is also active on the PMC (Guarneri et al., Drugs of Today, 30: 91, 1994), or anticholinergic compounds such as oxybutynin (Andersson, Drugs 36: 477, 1988) and tolterodine (Nilvebrant, Life Sci. 68: 2549, 2001). The use of α₁-adrenergic receptor antagonists for the treatment of BPH is common too, but is based on a different mechanism of action (Lepor, Urology, 42: 483, 1993). However, treatments that involve direct inhibition of the pelvic musculature (including the detrusor muscle) may have unwanted side effects, such as incomplete voiding or accommodation paralysis, tachycardia and dry mouth (Andersson, Drugs 35: 477, 1988). Thus, it would be preferable to utilize compounds that act via the central nervous system to affect, for example, the sacral spinal reflex and/or the PMC inhibition pathways in a manner that restores normal functioning of the micturition mechanism.

The descending bulbospinal pathway to the urinary bladder is essentially an inhibitory circuit, with 5-HT as,a key neurotransmitter (deGroat et al., in: Neurophysiology of Micturition and Its Modification in Animal Models ofHuman Disease. Maggi C. A. Ed., Harwood Academic Publishers, pp. 227-290, 1993).

At least 15 different populations of 5-HT receptors have been identified. These receptor types belong to 5-HT receptor families 5-HT₁-5-HT₇, and several of the families are composed of subpopulations. For example, 5-HT₁ receptors are a family of 5-HT receptors that are negatively coupled to adenylate cyclase and consist of 5-HT_{1A}, 5-HT_{1B}, 5-HT_{1D}, 5-HT_{1E} and 5-HT_{1F} subtypes (Gerhardt van Heerikhuitzen, Eur. J. Pharmacol. 334: 1, 1997).

Of particular interest to the present invention are the 5-HT_{1A} and 5-HT_{1B} receptors. Animal and human 5-HT_{1A} receptors act as somatodendritic and presynaptic receptors on nerve cells thus modulating neural firing, and at the postsynaptic level where they mediate inhibitory functions. Certain rodent species, including rat and mouse, possess 5-HT_{1B} receptors that serve primarily as terminal autoreceptors. In humans, the corresponding receptors that function in a similar manner were initially termed 5-HT_{1Dβ} (Weinshank et al., Proc. Natl. Acad. Sci. USA 89: 3630, 1992; Artig et al., Mol. Pharmacol. 41: 1, 1992). Rat 5-HT_{1B} receptors and human 5-HT_{1Dβ} receptors are considered species homologues, and there is >90% transmembrane sequence homology between them. It has been recommended that human 5-HT_{1Dβ} receptors be termed h5-HT_{1B} receptors (Hartig et al., Trends Pharmacol. Sci. 17: 103, 1996). Most agents that bind at rat 5-HT_{1B} receptors also bind at human cloned 5-HT_{1B} receptors.

In the central nervous system, several independent serotonergic cell clusters located in the raphe nuclei have been identified, possessing differential projection patterns. Serotonin within the dorsal horn of spinal cord arises primarily from neurons in the pontomedullary-nucleus raphe magnum (NRM) (Bowker et al. Brain Res 226:187, 1981).

Raphe neurons are activated by bladder distension (Lumb Prog Brain Res 67: 279, 1986; Oh et al. Soc Neuroscience Abstracts 12: 375, 1986). Furthermore, electrical stimulation of 5-HT-containing neurons of NRM and activation of postsynaptic 5-HT receptors in the spinal cord of cats inhibit bladder contractions and reflex firing in the sacral efferent pathways to the bladder (Morrison and Spillane J Auton Nervous System Supp 393, 1986; Sugaya et al. J Urol 159: 2172, 1998). Stimulation of the NRM also inhibits the firing of spinal dorsal horn neurons activated by afferents in the pelvic nerve (Lumb Prog Brain Res 67: 279, 1986).

During the firing of raphe neurons, serotonin released within the raphe region from dendrites and possibly axon terminals (Chazal and Ralston J Comp Neurol 259: 317, 1987) acts on somatodentritic 5-HT_{1A} receptors to inhibit neuronal activity through a local negative feedback mechanism (Aghajanian and Vander-Maelen Handbook Physiol 4: 237, 1986). Neutral antagonists acting at somatodendritic 5-HT_{1A} receptors, in contrast, increased the firing rate of raphe nuclei cells of rats in vitro (Corradetti et al. J Pharmacol Exp Ther 278: 679, 1996), as well as in cats and guinea pigs in vivo (Fornal et al. J Pharmacol Exp Ther 270:1345, 1996; Mundey et al. Br J Pharmacol 117: 750, 1996).

Neutral antagonists at somatodendritic 5-HT_{1A} receptors therefore, by increasing the firing of NRM neurons, lead to an increase of spinal 5-HT thus inhibiting the micturition reflex (Testa et al. J Pharmacol. Exp. Ther 290: 1258, 1999). The release of 5-HT is inhibited by the stimulation of presynaptic 5-HT_{1B} receptors (induced by the 5-HT itself) which are located on the synaptic terminals of serotonergic neurons (Bolanos-Jimenex et al., Eur. J. Pharmacol. 294: 531, 1995). The 5-HT_{1B} subtype receptor has been localized in all laminae in the spinal cord, where they represent approximately 18% of all 5-HT binding sites. Most of these 5-HT_{1B} receptors are located on the terminals of descending pathways from raphe nuclei (Gjerstad et al., Eur. J Pharmacol. 335: 127, 1997).

The use of 5-HT_{1A} receptor antagonists in treatment of urinary incontinence (UI) or overactive bladder (OAB) is disclosed, e.g., in U.S. Pat. Nos. 6,399,614, 6,271,234, 6,071,920 and 5,990,114, herein incorporated by reference in their entireties.

Additional compounds that are 5-HT_{1A} receptor antagonists are disclosed in U.S. Pat. Nos. 6,514,976, 6,358,958, and 6,239,135, herein incorporated by reference in their entireties. The compounds are disclosed as useful for treatment of urinary incontinence. These patents, however, provide no experimental support for treatment of urinary incontinence, either in human patients or in an animal model for lower urinary tract disease.

WO 99/05134, WO 99/14207, WO 99/14212 and WO 99/14213, herein incorporated by reference in their entireties, disclose compounds as useful for the treatment of different diseases, including urinary incontinence. The compounds are disclosed as 5-HT_{1B} receptor antagonists. None of these documents, however, provide experimental support for 5-HT_{1B} receptor binding, or treatment of urinary incontinence, either in human patients or in an animal model for lower urinary tract disease.

WO 95/31988 discloses combinations of 5-HT_{1A} and 5-HT_{1D} antagonists and their methods of use in treating CNS disorders. The document does not disclose the use of a combination of 5-HT_{1A} and 5-HT_{1D} antagonists for the treatment of urinary incontinence, either in human patients or in an animal model for lower urinary tract disease.

WO 99/13876 discloses the use of the combination of one class of robalzotan-like 5-HT_{1A} antagonists and one class of 5-HT_{1B} antagonists or partial agonists for treatment of different diseases, including urinary incontinence. The reference does not, however, provide experimental support for treatment of urinary incontinence, either in human patients or in an animal model for lower urinary tract disease.

Accordingly, none of the aforementioned documents provides support for treatment of urinary incontinence, either in human patients or in an animal model for lower urinary tract disease.

Patients with lower urinary tract conditions often respond to certain classes or subclasses of therapeutic agents. Furthermore, patients may respond initially to a therapeutic agent, but become non-responsive to the agent overtime. Additionally, patients may exhibit undesirable side effects when therapeutic agents are administered in concentrations required to treat lower urinary tract conditions. These side effects may be overcome by administering lower dosages of two or more therapeutic agents to achieve a therapeutic effect, wherein one or more of the lower dosages would not be sufficient to have a therapeutic when the respecitve therapeutic agent is used in monotherapy.

Accordingly, one of ordinary skill in the art will appreciate a continuing need to identify new treatment regimens for treatments of lower urinary tract disease(s). The new treatment regimens may include, for example, combination therapies that target two or more receptors involved in lower urinary tract conditions.

Accordingly, the present inventors have unexpectedly found that administration of a combination of compounds at least one of which is endowed with antagonistic activity at 5-HT_{1A} and at least one of which is endowed with antagonist activity at 5-HT_{1B} receptors, or a compound that has both 5-HT_{1A} and 5-HT_{1B} antagonistic activity, produces a synergistic effect and as such provides a very potent inhibition of the micturition reflex.

### SUMMARY OF THE INVENTION

The invention is based on the finding that combination therapy with 5-HT_{1A} and 5-HT_{1B} antagonists is useful in the treatment of neuromuscular dysfunction of the lower urinary tract in mammals.

Thus, the invention provides methods for treating neuromuscular dysfunction of the lower urinary tract in mammals, including without limitation, dysuria, incontinence, and enuresis. The methods involve administering to affected mammals in need of treatment of neuromuscular dysfunction of the lower urinary tract, an effective amount of a one or more compounds having antagonistic activity at 5-HT_{1A} and/or 5-HT_{1B} receptors. In certain embodiments, the methods involve administering a compound that has antagonist activity at both of 5-HT_{1A} and 5-HT_{1B} receptors. In other embodiments, compounds having antagonist activity at only one of 5-HT_{1A} or 5-HT_{1B} receptors are administered in combination, such that at least one compound having antagonist activity at 5-HT_{1A} receptor is administered in combination with at least one compound having antagonist activity at 5-HT_{1B} receptor.

In preferred embodiments, a compound having 5-HT_{1A} antagonist activity has a structure represented by formulas A-K below.

Formula A, disclosed in U.S. Pat. Nos. 6,071,920, 6,399,614 and U.S. Patent Application Publication No. U.S.-2002-0193383, hereby incorporated by reference in their entireties, disclose compounds of the form

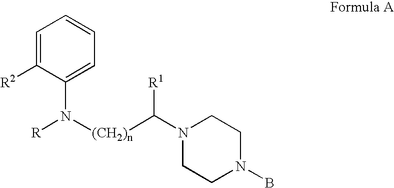

wherein for Formula A

R is a hydrogen atom, or alkylcarbonyl, cycloalkylcarbonyl, cycloalkylcarbonyl group substituted with one or more lower alkyl group or acyl group, or a monocyclic heteroarylcarbonyl group,

R¹ is a hydrogen atom or a lower alkyl group,

R² is an alkoxy, phenoxy, nitro, cyano, acyl, amino, acylamino, alkylsulphonylamino, alkoxycarbonyl, aminocarbonyl, N-alkylaminocarbonyl, N,N-dialkylaminocarbonyl, N-acylaminocarbonyl, halogen, trifluoromethyl or polyfluoroalkoxy group,

B is a mono- or bi-cyclic aryl, each optionally substituted with one or more lower alkyl, lower alkoxy, polyhaloalkoxy, halogen, hydroxyl, nitro, cyano, amido, amino, alkylamino, acylamino, alkylsulphonylamino, lower acyloxy, lower N-alkylaminocarbonyloxy, N,N-dialkylaminocarbonyloxy or acyl group, a mono- or bicyclic heteroaryl, each optionally substituted with one or more alkyl, alkoxy, halogen, nitro, cyano, amido, amino, alkylamino, acylamino, alkylsulphonylarnino or acyl group, or benzyl, optionally substituted with one or more alkyl, alkoxy, halogen, nitro, cyano, amido, amino, alkylamino, acylamino, alkylsulphonylamino, or acyl group,

and n is 1 or 2,

or a pharmaceutically acceptable salt, enantiomer, diastereomer, N-oxide, crystalline form, hydrate, solvate, active metabolite or prodrug thereof.

A preferred compound of Formula A is 1-[N-(2-nitrophenyl)-N-cyclohexylcarbonyl-2-aminoethyl]-4-(2-methoxyphenyl)piperazine.

Formula B, disclosed in U.S. Pat. No. 6,271,234, herein incorporated by reference in its entirety, discloses compounds of the form:

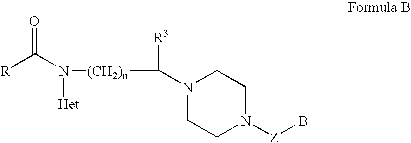

wherein for Formula B

n is 1 or 2,

Het is a monocyclic heteroaryl group,

R is a cycloalkyl or a monocyclic heteroaryl group,

R³ is a hydrogen atom or a lower alkyl group,

Z is a bond, —CH₂—, —CH₂CH₂—, —CH₂C(O)—, —CH₂CH(OH)—, —O—, —OCH₂—

or —C(O)— group, each of which is depicted with its left end being the end which attaches to the piperazine ring and the right end being the end which attaches to group B,

B is selected from the group consisting of a heteroaryl, unsubstituted aryl, and substituted aryl groups, where substituted aryl is represented by the formula

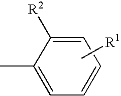

where R¹ is a single substituent selected from the group consisting of hydrogen, alkoxy, halogen, nitro, amino, acylamino, alkylamino, dialkylamino and alkylsulfonylamino, and R² is selected from the group consisting of alkoxy, polyfluoroalkoxy, cyano, halogen and aminocarbonyl,

and where the heteroaryl radical is selected from the group consisting of a mono or a bicyclic aromatic ring comprising from 5 to 12 ring atoms, where one or more of the ring atoms are selected from the group consisting of nitrogen, oxygen, and sulfur,

or a pharmaceutically acceptable salt, enantiomer, diastereomer, N-oxide, crystalline form, hydrate, solvate, active metabolite or prodrug thereof.

Further preferred are compounds of formula B wherein

n is 1 or 2,

Het is pyridine,

R is a cycloalkyl or a monocyclic heteroaryl group, wherein the monocyclic heteroaryl group is an aromatic group consisting from 5 to 6 ring atoms, and one or two of said ring atoms is a member selected from the group consisting of nitrogen, oxygen and sulfur,

R³ is a hydrogen atom or a lower alkyl group,

Z is a —CH₂—,

wherein B is selected from the group consisting of heteroaryl and substituted aryl groups, wherein said substituted aryl group is represented the following formula:

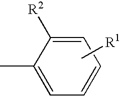

wherein R¹ is a single substituent selected from the group consisting of alkoxy, halogen, nitro, amino, acylamino, alkylamino, and alkylsulfonylamino, and R² is selected from the group consisting of alkoxy, polyfluoroalkoxy, cyano, halogen and aminocarbonyl, and wherein said heteroaryl group is selected from the group consisting of a monocyclic aromatic group consisting of 5 or 6 ring atoms, wherein one or more of said ring atoms are selected from the group consisting of nitrogen, oxygen, and sulfur, and wherein said acylamino is selected from the group consisting of acetylamino, pivaloylamino, butanoylamino, phenylacetylamino, and formylamino,

or a pharmaceutically acceptable salt, enantiomer, diastereomer, N-oxide, crystalline form, hydrate, solvate, active metabolite or prodrug (or a pharmaceutically acceptable salt) thereof.

Formula C, disclosed in U.S. Pat. No. 6,514,976, incorporated herein by reference in its entirety, discloses compounds of the form

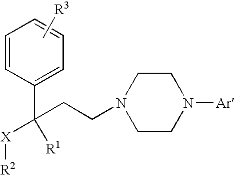

wherein for Formula C

Ar′ is a mono- or bi-cyclic aryl or heteroaryl radical, each of which may be optionally substituted independently with one to three substituents selected from the group consisting of hydrogen, C₁₋₆-alkyl, C₁₋₆-alkoxy, C₁₋₆-alkylthio, C₂₋₆-alkenyl, C₂₋₆-alkynyl, C₁₋₆-alkylhalo, C₃₋₈-cycloalkyl, C₃₋₈-cycloalkenyl or halo;

R¹ is hydrogen, C₁₋₆-alkyl, C₁₋₆-alkoxy, or C₁₋₆-alkylthio;

R² is phenyl, naphthyl or C₃-1₂-cycloalkyl, each of which may be optionally substituted independently with one or two substituents selected from the group consisting of hydrogen, C₁₋₆-alkyl, C₁₋₆-alkoxy, C₁₋₆-alkylthio, C₂₋₆-alkenyl, C₂₋₆-alkynyl, C₁₋₆-alkylhalo, C₃₋₈-cycloalkyl, C₃₋₈cycloalkenyl and halo;

R³ is selected from the group consisting of hydrogen, C₁₋₆-alkyl, C₁₋₆-alkoxy, C₁₋₆-alkylthio, C₂₋₆-alkenyl, C₂₋₆-alkynyl, C₁₋₆-alkylhalo, C₃₋₈-cycloalkyl, C₃₋₈-cycloalkenyl and halo;

X is —C(═O)—, —CHOH— or —CH₂—;

or a pharmaceutically acceptable salt, enantiomer, diastereomer, N-oxide, crystalline form, hydrate, solvate, active metabolite or prodrug thereof.

Formula D, disclosed in U.S. patent application Ser. No. 10/463,196, published as U.S. Patent Publication U.S. 2004/0072839 A1, incorporated herein by reference in its entirety, discloses compounds of the form:

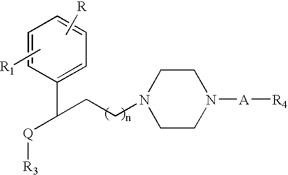

wherein for Formula D

R represents hydrogen or one or more substituents selected from the group consisting of (C₁-C₆)-alkyl, (C₁-C₆)-alkoxy, (C₁-C₆)-alkylthio, hydroxy, halo, (C₂-C₆)-alkenyl, (C₂-C₆)-alkynyl, (C₁-C₆)-haloalkyl, (C₁-C₆)-haloalkoxy, (C₁-C₆)-hydroxyalkyl, alkoxyalkyl, nitro, amino, (C₁-C₆)-aminoalkyl, (C₁-C₆)-alkylamino-(C₁-C₆)-alkyl, (C₁-C₆)-alkylamino, di-(C₁-C₆)-alkylamino, acylamino, (C₁-C₆)-alkylsulphonylamino, aminosulphonyl, (C₁-C₆)-alkylaminosulphonyl, cyano, aminocarbonyl, N-(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups;

R₁ represents a member selected from the group consisting of hydrogen, cycloalkyl, aryl, aryloxy, aralkyl, aralkoxy, heterocyclic, heterocycloxy, heterocycloalkyl and heterocycloalkoxy groups, each group being optionally substituted with one or more substituent R, defined as above;

Q represents —C(O)— or —CH(OR₂)— where R₂ represents a member selected from the group consisting of hydrogen, (C₁-C₆)-alkyl, (C₂-C₆)-alkenyl, (C₂-C₆)-alkynyl and cycloalkyl groups, wherein each group is optionally substituted with one or more groups selected from R₅ and R₆, where R₅ is selected from the group consisting of halo, (C₁-C₆)-alkoxy, (C₁-C₆)-haloalkoxy, cyano, (C₁-C₆)-alkoxycarbonyl, (C₁-C₆)-alkylcarbonyl, alkoxyalkyl, aminocarbonyl, N-(C₁-C₆)-alkylaminocarbonyl, N,N-di-(C₁-C₆)-alkylaminocarbonyl groups and R₆ is selected from the group consisting of aryl, heteroaryl, aryloxy, heteroaryloxy, arylalkoxy, and heteroarylalkoxy groups, each optionally substituted with R, or R₂ represents —C(O)— (C₁-C₆)-alkyl, —C(O)O—(C₁-C₆)-alkyl, —C(O)NR₇R₈ or —C(S)NR₇R₈ wherein R₇ and R₈ are independently hydrogen or (C₁-C₆)-alkyl;

R₃ represents hydrogen or a (C₁-C₆)-alkyl, (C₂-C₆)-alkenyl, (C₂-C₆)-alkynyl, cycloalkyl, aryl or heterocycle group, each group being optionally substituted with one or more substituent R or R₁, defined as above;

R₄ represents an aryl or heterocyclic group, each being optionally substituted with one or more substituent R, defined as above;

A represents a bond or (CH₂)ₙ; and

n=1 or 2,

or an enantiomer, optical isomer, diastereomer, N-oxide (e.g., N-piperazine oxide), crystalline form, hydrate, solvate or pharmaceutically acceptable salt thereof.

As referred to in the definition of R₆, aryl, heteroaryl, aryloxy, heteroaryloxy, arylalkoxy and heteroarylalkoxy group may be optionally substituted with one or more substituents selected from the group consisting of, (C₁-C₆)-alkyl, (C₁-C₆)-alkoxy, (C₁-C₆)-alkylthio, hydroxy, halo, (C₂-C₆)-alkenyl, (C₂-C₆)-alkynyl, (C₁-C₆)-haloalkyl, (C₁-C₆)-haloalkoxy, (C₁-C₆)-hydroxyalkyl, alkoxyalkyl, nitro, amino, (C₁-C₆)aminoalkyl, (C₁-C₆)-alkylamino(C₁-C₆)-alkyl, (C₁-C₆)-alkylamino, di(C₁-C₆)-alkylamino, acylamino, (C₁-C₆)-alkylsulphonylamino, aminosulphonyl, (C₁-C₆)-alkylaminosulphonyl, cyano, aminocarbonyl, N-(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, formyl, alkylcarbonylalkyl, alkanoyloxyalkyl, (C₁-C₆)-alkylaminocarbonylamino, (C₁-C₆)-alkylsulphinyl, (C₁-C₆)-alkylsulphonyl, and N, N-di-(C₁-C₆)-alkylaminosulphonyl groups.

Formula E, disclosed in U.S. patent application Ser. No. 10/463,222, published as U.S. Patent Publication U.S. 2004/0072822 A1, incorporated herein by reference in its entirety, describes compounds of the form

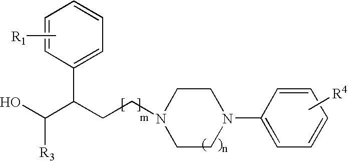

wherein for Formula E

R¹ represents a halogen atom,

R³ represents a (C₃-C₈)-cycloalkyl group,

R⁴ represents a (C₁-C₄)-alkoxy or (C₁-C₄)-haloalkoxy group,

m is 1 or 2, and

n is 1 or 2,

or a pharmaceutically acceptable salt, enantiomer, diastereomer, N-oxide, crystalline form, hydrate, solvate, active metabolite or prodrug (or a pharmaceutically acceptable salt) thereof.

Formula F, disclosed in U.S. patent application Ser. No. 10/463,221, published as U.S. Patent Publication U.S. 2004/0058962 A1, incorporated herein by reference in its entirety, describes compounds of the form

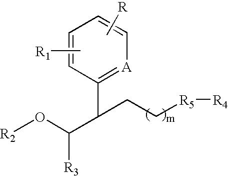

wherein for formula F

R is hydrogen or one or more substituents selected from the group consisting of alkyl, alkoxy, alkylthio, hydroxy, halo, alkenyl, alkynyl, polyhaloalkyl, monohaloalkoxy, polyhaloalkoxy, hydroxyalkyl, alkoxyalkyl, nitro, amino, aminoalkyl, alkylaminoalkyl, alkylamino, dialkylamino, acylamino, alkylsulphonylamino, aminosulphonyl, alkylaminosulphonyl, cyano, aminocarbonyl, N-alkylaminocarbonyl, N,N-dialkylaminocarbonyl, alkoxycarbonyl, alkylcarbonyl, alkylcarbonylalkyl, formyl, alkanoyloxyalkyl, alkylaminocarbonylamino, alkylsulphinyl, alkylsulphonyl, and N,N-dialkylaminosulphonyl groups;

R¹ is selected from the group consisting of hydrogen, cycloalkyl, aryl, aryloxy, aralkyl, aralkoxy, heterocyclic, heterocycloxy, heterocycloalkyl and heterocycloalkoxy groups, each group being optionally substituted with one or more substituent R, defmed as above;

R² is selected from the group consisting of hydrogen, alkyl, alkenyl, alkynyl and cycloalkyl groups, wherein each group is optionally substituted with one or more groups selected from R⁸ and R⁹, where R⁸ is selected from the group consisting of halo, alkoxy, monohaloalkoxy, polyhaloalkoxy, cyano, alkoxycarbonyl, alkylcarbonyl, alkoxyalkyl, aminocarbonyl, N-alkylaminocarbonyl, N,N-dialkylaminocarbonyl groups and R₉ is selected from the group consisting of aryl, heteroaryl, aryloxy, heteroaryloxy, arylkoxy, and heteroarylkoxy groups, each optionally substituted with R¹;

R³ is alkyl, alkenyl, alkynyl, cycloalkyl, aryl or heterocycle, each being optionally substituted with one or more substituent R or R¹, defined as above;

R⁴ is aryl or heterocyclic, each being optionally substituted with one or more substituents R, defined as above;

A is CH or N,

R⁵ is 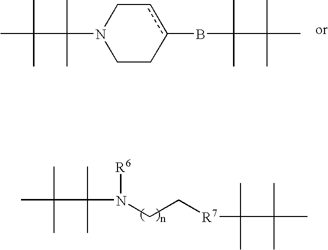
(where R⁴ is bound to the right of each group)

m and n are independently 1 or 2,

R⁶ is H or alkyl,

R⁷ is O, S, NR⁶ or CH₂; and

B is a bond, O, S, NR⁶ or CH₂; and 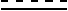
is a single or double bond,

or a pharmaceutically acceptable salt, enantiomer, diastereomer, N-oxide, crystalline form, hydrate, solvate, active metabolite or prodrug thereof.

Formula G, disclosed in published U.S. Patent Publications No. 2003/0181446 A1 and 2003/0162777 A1, incorporated herein by reference in their entireties, describe compounds of the form

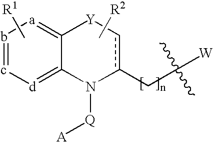

wherein for Formula G

W represents

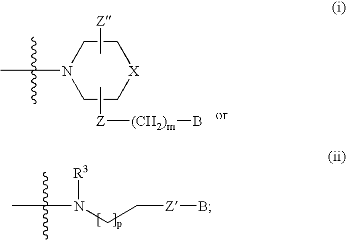

R¹ is one or more substituents selected from a group consisting of hydrogen, halogen, hydroxyl, alkyl, substituted alkyl, alkoxyl, substituted alkoxyl, nitro, aryl, substituted aryl, heterocycle, substituted heterocycle, alkenyl, substituted alkenyl, amino, alkylamino, dialkylamino, cyano, —SR³, —C(O)R³, —C(O)NR³R³, —NR³C(O)R³, —NR³SO₂R³, —NR³C(O)OR³ and —N(H)C(O)N(H)R³;

R³ is independently selected from a group consisting of hydrogen, alkyl, substituted alkyl, cycloalkyl, substituted cycloalkyl, aryl, substituted aryl, heterocycle and substituted heterocycle;

R² is one or two substituents selected from a group consisting of hydrogen, halogen, oxo, alkyl, substituted alkyl, alkenyl and substituted alkenyl groups;

Y represents a CH, CH₂, CR₂, CHR₂ group or a bond;

Q represents a carbonyl, thiocarbonyl or sulfonyl group;

A represents an alkyl, substituted alkyl, cycloalkyl, substituted cycloalkyl, alkenyl, substituted alkenyl, cycloalkenyl, substituted cycloalkenyl, aryl, substituted aryl, heterocycle, substituted heterocycle, alkylamino, substituted alkylamino, dialkylamino, substituted dialkylamino, cyclic amino, substituted cyclic amino, arylamino, substituted arylamino, arylalkylamino or substituted arylalkylamino group;

n is independently 1 or 2;

m is independently 0, 1 or 2;

p is independently 1, 2 or 3;

a, b, c and d are independently a carbon or nitrogen atom, or CH, CH₂ or NH group, with the proviso that no more than two of a, b, c and d may simultaneously be a nitrogen atom and/or NH,

X represents a bond, CH, CH₂, SO or SO₂ group or a carbon, nitrogen or sulphur atom and, when X is a nitrogen atom or CH group, the -Z-(CH₂)ₘ—B group is bound to said nitrogen atom or CH group, and when X is a carbon atom Z″ is not a hydrogen atom or oxo group and the Z-(CH₂)ₘ—B and Z″ groups are bound to said carbon;

Z represents a bond, an oxygen or sulphur atom or —CH(OH)—, —C(O)—NR³C(O)—, —NR³—C(O)—NR³—, or —NR³— group;

Z′ represents a bond or an oxygen or sulphur atom;

Z″ represents a hydrogen atom or hydroxyl, oxo, alkylcarbonyl or cyano group,

B represents a monocyclic aryl, substituted monocyclic aryl, bicyclic aryl, substituted bicyclic aryl, monocyclic heterocycle, substituted monocyclic heterocycle, bicyclic heterocycle or substituted bicyclic heterocycle; 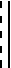
represents a single or double bond and, when Y═CH, the double bond is shifted so as to contain it; and

the term “substituted” for Formula G without further description refers to the instance where one or more hydrogen atoms on a radical are replaced independently with one or more atoms or groups selected from halogen, hydroxyl, oxo, nitro, cyano, alkyl, haloalkyl, polyhaloalkyl, alkylthio, alkoxyalkyl, alkenyl, alkynyl, cycloalkyl, cycloalkenyl, cycloalkynyl, alkoxyl, alkenyloxyl, alkynyloxyl, cycloalkoxyl, aryloxyl, substituted aryloxyl, cycloalkenyloxyl, cycloalkynyloxyl, arylalkoxyl, acyloxyl, alkylaminocarbonyloxyl, sulphonyloxyl, polyhaloalkylsulphonyloxyl, acyl, ureido, amino, alkylamino, dialkylamino, acylamino, diacylamino, N-alkyl-N-aroylamino, N-arylkyl-N-alkylsulphonylamino, alkylsulphonylamino, alkenylamino, dialkenylamino, arylamino, diarylamino, alkoxycarbonylamino, alkoxycarbonyl, acylamino, acylalkylamino, sulphonylamino, sulphonylalkylamino, cyanoamino, arylsufonyl, alkylarylsulfonyl, sulfamoyl, aryl, substituted aryl, arylalkylamino, substituted arylalkylamino, heterocycle, substituted heterocycle, aralkyl, aryloxyalkyl, heterocycloxyalkyl, heterocyclicalkyl, wherein the terms substituted heterocycle, substituted aryl, substituted aryloxyl and substituted arylalkylamino refer respectively to a heterocyclic, aryl, aryloxyl or arylalkylamino group wherein one or more of the hydrogen atoms on a ring of the heterocyclic, aryl, aryloxyl or arylalkylamino group is replaced by one or more of the substituents recited herein, with the proviso that if variable A or B is substituted with a first substituted heterocycle, substituted aryl, substituted aryloxyl or substituted arylalkylamino and said first substituted heterocycle, substituted aryl, substituted aryloxyl or substituted arylalkylamino is substituted with a second substituted heterocycle, substituted aryl, substituted aryloxyl and substituted arylalkylamino, said second substituted heterocycle, substituted aryl, substituted aryloxyl and substituted arylalkylamino may not be substituted with a third substituted heterocycle, substituted aryl, substituted aryloxyl and substituted arylalkylamino,

or a pharmaceutically acceptable salt, enantiomer, diastereomer, N-oxide, crystalline form, hydrate, solvate, active metabolite or prodrug (or a pharmaceutically acceptable salt) thereof.

Formula H, disclosed in published U.S. patent application Ser. No. 09/127,059, incorporated herein by reference in its entirety, describes compounds of the form

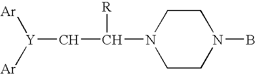

wherein for Formula H

each of Ar and Ar′ is independently selected from a group consisting of phenyl and pyridyl, each optionally substituted by one or more members selected from the group consisting of alkyl, alkoxy, cyano, nitro, amino, alkylsulfonylamino, and alkylamino;

Y is a member selected from the group consisting of nitrogen atom, CH, C—OH, C—CN and C—CONH₂;

R is a hydrogen atom or a lower alkyl group; and

B is (a) phenyl substituted with one or more substituents selected from the group consisting of alkoxy, halogen, cyano, nitro, amino, alkylsulfonylamino and alkylamino; (b) naphthyl, optionally substituted with one or more substituents selected from the group consisting of alkyl, alkoxy, halogen, cyano, nitro, amino, alkylsulfonylamino and alkylamino groups; (c) benzodioxanyl; or (d) indolyl,

or a pharmaceutically acceptable salt, enantiomer, diastereomer, N-oxide, crystalline form, hydrate, solvate, active metabolite or prodrug (or a pharmaceutically acceptable salt) thereof. A preferred compound of formula H is 1-[3-hydroxy-3,3 bis-(2-pyridyl)propyl]-4-(4-indolyl)piperazine.

Formula I, disclosed in WO 94/21610, incorporated herein by reference in its entirety, discloses compounds of the form

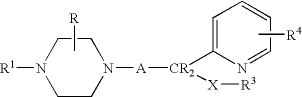

and the pharmaceutically acceptable acid addition salts thereof;

wherein for Formula I

R represents hydrogen or one or two same or different C₁₋₆-alkyl groups;

R¹ is a mono- or bicyclic aryl or heteroaryl radical;

R₂ is hydrogen or lower alkyl;

R³ is lower alkyl or cycloalkyl;

R⁴ is hydrogen or lower alkyl;

A is an alkylene chain of 1 to 3 carbon atoms optionally substituted by one or more lower alkyl groups; and

X is —CO—, —CR⁵OH— (where R⁵ is hydrogen, lower alkyl or cycloalkyl), —S—, —SO— or —SO₂— or X can also be —(CH₂)ₙ— (where n is 0, 1 or 2) when R³ is cycloalkyl.

Formula J, disclosed in U.S. Pat. No. 6,127,357, incorporated herein by reference in its entirety, discloses compounds of the form

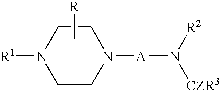

and the pharmaceutically acceptable acid addition salts thereof;

wherein for Formula J

A is an alkylene chain of 2 to 4 carbon atoms optionally substituted by one or more lower alkyl groups,

Z is oxygen or sulphur,

R is hydrogen or lower alkyl,

R¹ is a mono or bicyclic aryl or heteroaryl radical,

R² is a mono or bicyclic heteroaryl radical, and

R³ is hydrogen, lower alkyl, cycloalkyl, cycloalkenyl, cycloalkyl(lower)alkyl, aryl, aryl(lower)alkyl, heteroaryl, heteroaryl-(lower)alkyl, a group of formula —NR⁴R⁵, where R⁴ is hydrogen, lower alkyl, aryl or aryl-(lower)alkyl and R⁵ is hydrogen, lower alkyl, —CO(lower)alkyl, aryl, COaryl, aryl(lower)alkyl, cycloalkyl or cycloalkyl-(lower)alkyl or R⁴ and R⁵ together with the nitrogen atom to which they are attached represent a saturated heterocyclic ring which may contain a further hetero atom or R³ is a group of formula OR⁶, where R⁶ is lower alkyl, cycloalkyl, cycloalkyl(lower)alkyl, aryl, aryl(lower)alkyl, heteroaryl or heteroaryl(lower)alkyl.

Formula K, disclosed in U.S. Pat. No. 5,462,942, incorporated herein by reference in its entirety, discloses compounds of the form

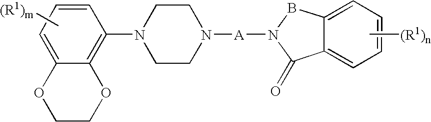

wherein for Formula K

R¹ is halogen, lower alkyl or alkoxy, hydroxy, trifluoromethyl or cyano,

m has the value 1 or 2,

n has the value 0 or 1,

A represents a C₂₋₆ alkylene chain which may be substituted with one more R substituent selected from the group consisting of lower alkyl and monocyclic (hetero)aryl groups, and

B is methylene, ethylene, carbonyl, sulfinyl, sulfonyl, or sulfur, and salts thereof.

A preferred compound of formula K is 2-[4-[4-(7-chloro-2,3-dihydro-1,4-benzodioxin-5-yl)piperazin-1-yl]butyl]1-2-benzisothiazol-3(2H)-one 1,1-dioxide.

In preferred embodiments, a compound having 5-HT_{1B} antagonist activity has a structure represented by formulas L to S below.

Formula L, disclosed in U.K. Patent Application GB 2 276 163, incorporated herein by reference in its entirety, discloses compounds of the form

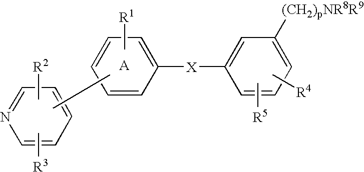

wherein for Formula L

R¹ represents a hydrogen or halogen atom, or C₁₋₆-alkyl, or C₁₋₆-alkoxy group;

R² and R³ independently represent a hydrogen or halogen atom, or a C₁₋₆-alkyl, hydroxyC₁₋₆-alkyl, C₁₋₆-alkoxyC₁₋₆-alkyl, C₁₋₆-alkoxy, hydroxy, —CN, —NO₂, —CO₂R⁶, —COR⁶, —C(O)NR⁶R⁷, OR —(CH₂)ₘOC(O)Cl₄alkyl group;

R⁴ and R⁵ independently represent a hydrogen or halogen atom, or a hydroxy, C₁₋₆-alkyl, or C₁₋₆-alkoxy group;

R⁶, R⁷, R⁸, and R⁹ independently represent a hydrogen atom or a C₁₋₆-alkyl group;

or —NR⁶R⁷ forms a saturated heterocyclic ring which has 5 or 6 members which, when there are 6 ring members, may optionally contain in the ring one oxygen or sulfur atom;

X represents —C(O)NH—, —NHC(O)—, —CH₂NH— or —NHCH₂—;

m represents zero or an integer from 1 to 3; and

p represents an integer from 2 to 4,

or a pharmaceutically acceptable salt, enantiomer, diastereomer, N-oxide, crystalline form, hydrate, solvate, active metabolite or prodrug thereof.

A preferred compound of formula L is 3-[3-(dimethylamino)propyl]-4-hydroxy-N-[4-(4-pyridinyl)phenyl]benzamide.

Formula M, disclosed in U.S. Pat. No. 5,968,954, incorporated herein by reference in its entirety, describes compounds of the form

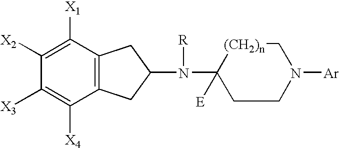

wherein for Formula M

n represents 1 or 2;

Ar represents

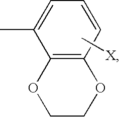

wherein X represents a hydrogen or fluorine atom, or Ar represents

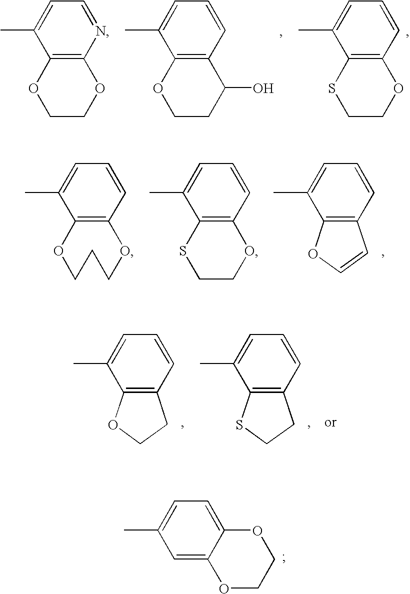

R represents a hydrogen atom, or C₁₋₅-alkyl, or aralkyl group,

E represents a hydrogen atom or methyl group, and

X₁, X₂, X₃, and X₄ independently represent a hydrogen or halogen atom, or C₁-C₅-alkyl, C₁-C₅-alkoxy, trifluoromethyl, hydroxy, cyano, nitro, —NR¹R , —C(O)NR¹R², —COOR³, —OC(O)R⁴,

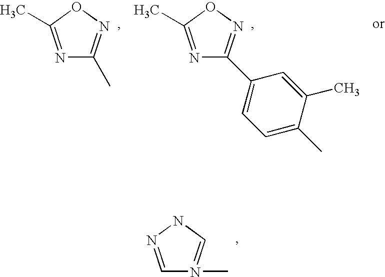

R¹, R², and R³ independently represent a hydrogen atom or C₁-C₅-alkyl group, and R⁴ represents a C₁-C₅-alkyl group,

or, independently, a pair of X₁ and X₂, X₂ and X₃, or X₃ and X₄, together with the carbon atoms of the phenyl ring to which they are attached, form a 5-membered or 6-membered ring composed of atoms selected from the atoms carbon, oxygen, nitrogen, and sulfur,

or a pharmaceutically acceptable salt, enantiomer, diastereomer, N-oxide, crystalline form, hydrate, solvate, active metabolite or prodrug thereof.

Additional compounds of formula M are disclosed in WO 02/074764.

Formula N, disclosed in WO 97/17350, incorporated herein by reference in its entirety, describes compounds of the form

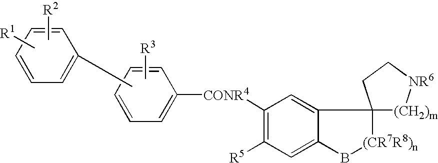

wherein for Formula N

R¹ represents a hydrogen or halogen atom, or C₁₋₆-alkyl, C₃₋₆-cycloalkyl, COC₁₋₆-alkyl, C₁₋₆-alkoxy, hydroxy, hydroxyC₁₋₆-alkyl, hydroxyC₁₋₆-alkoxy, C₁₋₆-alkoxyC₁₋₆alkoxy, acyl, nitro, trifluoromethyl, cyano, SR⁹, SOR⁹, SO₂R⁹, NR⁹CONR¹⁰R¹¹, NR¹⁰SO₂R¹¹, SO₂NR¹⁰R¹¹, CO₂R¹⁰, CONR¹⁰R¹¹, CO₂NR¹⁰R¹¹, CONR¹⁰(CH₂)ₐCO₂R¹¹, (CH₂)ₐNR¹⁰ R¹¹, (CH₂)ₐCONR¹⁰R¹¹, (CH₂)ₐNR¹⁰ COR¹¹, (CH₂)ₐCO₂C₁₋₆-alkyl, CO₂(CH₂)ₐOR¹⁰, NR¹⁰R¹¹, N═CNR⁹NR¹⁰R¹¹, NR¹⁰CO(CH₂)ₐNR¹⁰R¹¹, NR¹⁰CO₂R¹¹, CONHNR¹⁰R¹¹, CR¹⁰═NOR¹¹, CNR¹⁰═NOR¹¹, where R⁹, R¹⁰, and R¹¹ are independently hydrogen or C₁₋₆-alkyl and “a” is an integer from 1 to 4; or R¹ is a 5- to 7-membered heterocyclic ring containing 1 to 4 heteroatoms selected from oxygen, nitrogen, and sulfur, optionally substituted with one or more substituents defined as R² or R³ below;

R² and R³ are independently hydrogen, halogen, C₁₋₆-alkyl, C₃₋₆-cycloaklyl, C₃₋₆-cycloalkenyl, C₁₋₆-alkoxy, hydroxyC₁₋₆-alkyl, C₁₋₆-alkylOC₁₋₆-alkyl, acyl, aryl, acyloxy, hydroxy, nitro, trifluoromethyl, cyano, CO₂R¹⁰, CONR¹⁰R¹¹, NR¹⁰OR¹¹, where R¹⁰ and R¹¹ are independently hydrogen or C₁₋₆-alkyl;

R⁴ is hydrogen or C₁₋₆-alkyl;

R⁵ is hydrogen or C₁₋₆-alkyl, or R⁴ and R₅ together from a group -A-, where A is (CR¹³R¹⁴)q where q is 2, 3, or 4, and R¹³ and R¹⁴ are independently hydrogen or C₁₋₆-alkyl or A is (CR¹³R¹⁴)ᵣ-D where r is 0, 1, 2, or 3 and D is oxygen, sulfur, or CR¹³═CR¹⁴;

R⁶ is a group —(CH₂)ₚR¹⁵, where R¹⁵ is OR¹⁶ or SR¹⁶ where R¹⁶ is hydrogen or C₁₋₆-alkyl or R¹⁵ is NR¹⁰OR¹¹ where R¹⁰ and R¹¹ are as defined for R¹;

R⁷ and R⁸ are independently hydrogen or C₁₋₆-alkyl;

B is oxygen, CR¹⁷R¹⁸ or NR¹⁹ where R¹⁷, R¹⁸, and R¹⁹ are independently hydrogen or C₁₋₆-alkyl or B is a group S(O)_{b} where b is 1, 2, or 3;

m is 1, 2, or 3; and

n is 1, 2, or 3,

or a pharmaceutically acceptable salt, enantiomer, diastereomer, N-oxide, crystalline form, hydrate, solvate, active metabolite or prodrug thereof.

Formula O, disclosed in WO 97/17351, incorporated herein by reference in its entirety, describes compounds of the form

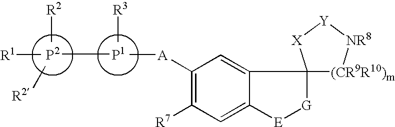

wherein for formula O

P¹ and P² are independently phenyl, bicyclic aryl, a 5- to 7-membered heterocyclic ring containing 1 to 4 heteroatoms selected from oxygen, nitrogen, or sulfur, or a bicyclic heterocyclic ring containing one to three heteroatoms selected from oxygen, nitrogen, or sulfur;

R¹ represents a hydrogen or halogen atom, or C₁₋₆-alkyl, C₃₋₆-cycloalkyl, COC₁₋₆-alkyl, C₁₋₆-alkoxy, hydroxy, hydroxyC₁₋₆-alkyl, hydroxyC₁₋₆-alkoxy, C₁₋₆-alkoxyC₁₋₆alkoxy, acyl, nitro, trifluoromethyl, cyano, SR⁹, SOR⁹, SO₂R⁹, SO₂NR¹⁰R¹¹, CO₂R¹⁰, NR¹⁰SO₂R¹¹, CONR¹⁰R₁₁, CO₂NR¹⁰R¹¹, CONR¹⁰(CH₂)ₚCO₂R¹¹, (CH₂)ₚNR¹⁰R¹¹, (CH₂)ₚCONR¹⁰R¹¹, (CH₂)ₚNR¹⁰COR¹¹, CONR¹⁰(CH₂)ₚCO₂C₁₋₆-alkyl, CO₂(CH₂)ₚOR¹⁰, CONHNR¹⁰R¹¹, NR¹⁰R¹¹, N═CNR⁹NR¹⁰R¹¹, NR¹⁰CO₂R¹¹, NR¹⁰CO(CH₂)ₚNR¹⁰R¹¹, NR¹⁰CONR¹⁰R¹¹, CR¹⁰═NOR¹¹, CNR¹⁰═NOR¹¹, or NR¹²COR¹³, where R⁹, R¹⁰, and R¹¹ are independently hydrogen or C₁₋₆-alkyl, p is 1 to 4, R¹² is hydrogen, C₁₋₆-alkyl or together with R² forms a group (CH₂)_{q} where q is 2, 3, or 4 and R1³ is hydrogen, C₁₋₆-alkyl, aryl, or aryl substituted with one or more substituents selected from R² and R³, as defined below; or RI is a 5- to 7-membered heterocyclic ring containing 1 to 4 heteroatoms selected from oxygen, nitrogen, or sulfur, optionally substituted with one or more substituents selected from R² and R³, as defined below;

R² and R³ are independently hydrogen, halogen, C₁₋₆-alkyl, C₃₋₆-cycloalkyl, C₃₋₆-cycloalkenyl, C₁₋₆-alkoxy, hydroxyC₁₋₆-alkyl, C₁₋₆alkylOC₁₋₆-alkyl, acyl, aryl, acyloxy, hydroxy, nitro, trifluoromethyl, cyano, CO₂R¹⁰, CONR¹⁰OR¹¹, NR¹⁰R¹¹ where R¹⁰ and R¹¹ are independently hydrogen or C₁₋₆-alkyl, or R² and R³ together form a group —(CH₂)ᵣ—R¹⁴—(CH₂)ₛ— where R¹⁴ is O, S, CH₂, or NR¹⁵ where R¹⁵ is hydrogen or C₁₋₆-alkyl and r and s are independently 0, 1, or 2;

A is a group DR⁶—C(═B)— or a group —C(═B)-DR⁶ where B is oxygen or sulfur and D is nitrogen, carbon or a CH group; and

R⁶ is hydrogen or C₁₋₆-alkyl and R⁷ is C₁₋₆-alkyl, Cil₆-alkoxy, or halogen, or R⁶ and R⁷ together form a group -M- where M is (CR¹⁶R¹⁷)t where t is 1, 2, or 3 and R¹⁶ and R¹⁷ are independently hydrogen or C₁₋₆-alkyl or M is (CR¹⁶R¹⁷)ᵤ-J wherein u is 0, 1, or 2 and J is oxygen, sulfur, CR¹⁶═CR¹⁷, CR¹⁶═N, or N═N;

R⁸ is hydrogen or C₁₋₆-alkyl;

R⁹ and R¹⁰ are independently hydrogen or C₁₋₆-alkyl;

E is oxygen, CR¹⁸R¹⁹, or NR²⁰ where R¹⁸, R¹⁹ and R²⁰ are independently hydrogen or C₁₋₆-alkyl or E is S(O)ᵥ where v is 0, 1, or 2;

G is C═O or CR²¹R²² where R²¹ and R²² are independently hydrogen or C₁₋₆-alkyl;

X and Y are independently CR⁹R¹⁰ where R⁹ and R¹⁰ are defmed as above; and m is 1, 2, or 3, provided that P¹ and P² are not both phenyl,

or a pharmaceutically acceptable salt, enantiomer, diastereomer, N-oxide, crystalline form, hydrate, solvate, active metabolite or prodrug thereof.

Formula P, disclosed in WO 01/23374, incorporated herein by reference in its entirety, describes compounds of the form:

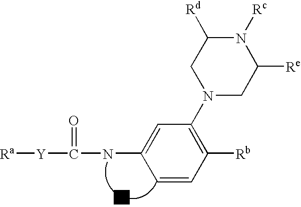

wherein for formula P

R^{a} is a group of formula (i)

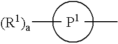

wherein

P¹ is phenyl, naphthyl, or heteroaryl;

R¹ is halogen, C₁₋₆-alkyl, C₃₋₆-cycloalkyl, COC₁₋₆-alkyl, C₁₋₆-alkoxy, hydroxy, hydroxyC₁₋₆-alkyl, nitro, trifluoromethyl, cyano, SR⁶, SOR⁶, S0₂R⁶, SO₂NR⁶R⁷, CO₂R⁶, CONR⁶R⁷, OCONR⁶R⁷, NR⁶R⁷, NR⁶CO₂R⁷, NR⁶CONR⁷R⁸, CR⁶═NOR⁷, where R⁶, R⁷ and R⁸ are independently hydrogen or C₁₋₆-alkyl;

a is 0, 1, 2 or 3;

or R^{a} is a group of formula (ii)

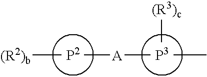

wherein

P² is phenyl, naphthyl, heteroaryl, or a 5- to 7-membered heterocyclic ring;

P³ is phenyl, naphthyl, or heteroaryl;

A is a bond or oxygen, carbonyl, CH₂ or NR⁴ where R⁴ is hydrogen or C₁₋₆-alkyl;

R² is as defined above for R¹ in formula (i) or R² is heteroaryl, optionally substituted by C₁₋₆-alkyl, halogen, or COC₁₋₆-alkyl, or is a 5- to 7-membered heterocyclic ring optionally subsituted by oxo;

R³ is halogen, C₁₋₆-alkyl, C₃₋₆-cycloalkyl, C₁₋₆-alkoxy, COC₁₋₆-alkyl, hydroxy, nitro, trifluoromethyl, cyano, CO₂R⁶, CONR⁶R⁷, NR⁶R⁷ where R⁶ and R⁷ are as defined above;

b and c are independently 0, 1, 2, or 3;

Y is a single bond, CH₂, O, or NR⁵ where R⁵ is hydrogen or C₁₋₆-alkyl;

W is —(CR⁹R¹⁰)ₜ— where t is 2, 3, or 4 and R⁹ and R¹⁰ are independently hydrogen or C₁₋₆-alkyl or W is a group CH═CH;

R^{b} is hydrogen, halogen, hydroxy, C₁₋₆-alkyl, trifluoromethyl, COC₁₋₆-alkyl, cyano or C₁₋₆-alkoxy;

R^{c} is hydrogen or C₁₋₆-alkyl; and

R^{d} and Re are independently C₁₋₄-alkyl,

or a pharmaceutically acceptable salt, enantiomer, diastereomer, N-oxide, crystalline form, hydrate, solvate, active metabolite or prodrug thereof.

Formula Q, disclosed in WO 02/074768, incorporated herein by reference in its entirety, describes compounds of the form

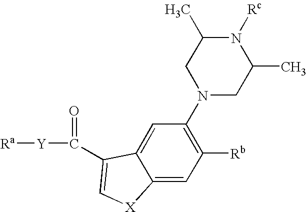

wherein for Formula Q

R^{a} is a group of formula (i)

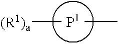

P¹ is phenyl, naphthyl, or heteroaryl;

R¹ is halogen, C₁₋₆-alkyl, C₃₋₆-cycloalkyl, COC₁₋₆-alkyl, C₁₋₆-alkoxy, hydroxy, hydroxyC₁₋₆-alkyl, nitro, haloC₁₋₆-alkyl, cyano, SR⁶, SOR⁶, SO₂R⁶, SO₂NR⁶R⁷, CO₂R⁶, CONR⁶R⁷, OCONR⁶R⁷, NR⁶R⁷, NR⁶CO₂R⁷, NR⁶CONR⁷R⁸, CR⁶═NOR⁷, where R⁶, R⁷and

R⁸are independently hydrogen or C₁₋₆-alkyl;

a is 0, 1, 2 or 3;

or R^{a} is a group of formula (ii)

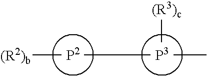

wherein

P² is phenyl, naphthyl, heteroaryl, or a 5- to 7-membered heterocyclic ring;

P³ is phenyl, naphthyl, or heteroaryl;

R² is as defined above for R¹ in formula (i) or R² is heteroaryl, optionally substituted by C₁₋₆-alkyl, halogen, or COC₁₋₆-alkyl, or is a 5- to 7-membered heterocyclic ring optionally subsituted by oxo;

R³ is halogen, C₁₋₆-alkyl, C₃₋₆-cycloalkyl, C₁₋₆-alkoxy, COC₁₋₆-alkyl, hydroxy, nitro, haloC₁₋₆-alkyl, cyano, CO₂R⁶, CONR⁶R⁷, NR⁶R⁷ where R⁶ and R⁷ are as defined above;

b and c are independently 0, 1, 2, or 3;

Y is a single bond, CH₂, or NH;

X is oxygen, sulfur, or N—R⁵ where R⁵ is hydrogen or C₁₋₆-alkyl;

R^{b} is hydrogen, halogen, C₁₋₆-alkyl, haloC₁₋₆-alkyl, COC₁₋₆-alkyl, or cyano; and

R^{c} is hydrogen or C₁₋₆-alkyl,

or a pharmaceutically acceptable salt, enantiomer, diastereomer, N-oxide, crystalline form, hydrate, solvate, active metabolite or prodrug thereof.

Formula R, disclosed in U.S. Pat. No. 5,801,170, incorporated herein by reference in its entirety, discloses compounds of the form

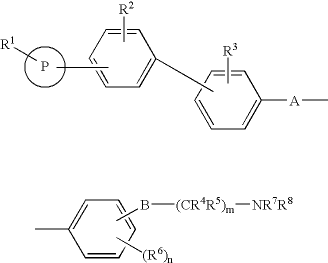

wherein for Formula R

P is a 5 to 7-membered heterocyclic ring containing 1 to 3 heteroatoms selected from oxygen, nitrogen or sulphur,

R¹, R² and R³ are independently hydrogen, halogen, C₁₋₆-alkyl, C₃₋₆-cycloalkyl, C₃₋₆-cycloalkenyl, C₁₋₆-alkoxy, hydroxy, C₁₋₆-alkyl, C₁₋₆-alkyl, OC₁₋₆-alkyl, acyl, aryl, acyloxy, hydroxy, nitro, trifluoromethyl, cyano, CO₂R⁹, CONR¹⁰R¹¹, NR¹⁰R¹¹ where R⁹, R¹⁰ and R¹¹ are independently hydrogen or C₁₋₆-alkyl;

R⁴ and R⁵ are independently hydrogen or C₁₋₆ alkyl;

R⁶ is hydrogen, halogen, hydroxy, C₁₋₆ alkyl or C₁₋₆ alkoxy;

R⁷ and R⁸ are independently hydrogen, C₁₋₆ alkyl, aralkyl, or together with the nitrogen atom to which they are attached form an optionally substituted 5- to 7-membered heterocyclic ring containing one or two heteroatoms selected from oxygen, nitrogen or sulphur;

A is CONH or NHCO;

B is oxygen, S(O)ₚ where p is 0, 1 or 2, NR¹² where R¹² is hydrogen, C₁₋₆-alkyl or phenylC₁₋₆-alkyl, or B is CR⁴═CR⁵ or CR⁴Rs where R⁴ and R⁵ are independently hydrogen or C₁₋₆-alkyl;

m is an integer from 1 to 4;

and n is an integer from 1 or 2;

or a salt thereof.

A preferred compound of formula R is N-[3-[2-(dimethylamino)ethoxy]-4-methoxyphenyl-2′methyl-4′-(5-methyl-1,2,4-oxadiazol-3-yl)-[1,1′ -biphenyl]-4-carboxamide.

Formula S, disclosed in U.S. Pat. No. 5,972,951, incorporated herein by reference in its entirety, discloses compounds of the form

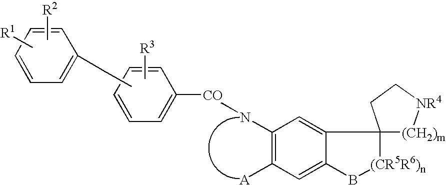

wherein for Formula S

R¹ is hydrogen, halogen, C₁₋₆-alkyl, C₃₋₆-cycloalkyl, COC₁₋₆-alkyl, C₁₋₆-alkoxy, hydroxy, hydroxyC₁₋₆alkyl, hydroxyC₁₋₆alkoxy, C₁₋₆alkoxyC₁₋₆alkoxy, acyl, nitro, trifluoromethyl, cyano, SR⁹, SOR⁹, SO₂R⁹, SO₂NR¹⁰R¹¹, CO₂R¹⁰, NR¹⁰SO₂R¹¹, CONR¹⁰R¹¹, CO₂NR¹⁰R¹¹, CONR¹⁰(CH₂)ₚCO₂R¹¹, (CH₂)ₚNR¹⁰R¹¹, (CH₂)ₚCONR¹⁰R¹¹, (CH₂)ₚNR¹⁰COR¹¹, (CH₂)ₚCO₂C₁₋₆alkyl, CO₂(CH₂)ₚOR¹⁰, CONHNR¹⁰R¹¹, NR¹⁰R¹¹, NR¹⁰CO₂R¹¹, NR¹⁰CO₂R¹¹, NR¹⁰CO(CH₂)ₚNR¹⁰R¹¹, NR¹⁰CONR¹⁰R¹¹, CR¹⁰═NOR¹¹, CNR¹⁰═NOR¹¹, where R⁹, R¹⁰ and R¹¹ are independently hydrogen or C₁₋₆alkyl and p is 1 to 4; or R¹ is an optionally substituted 5 to 7-membered heterocyclic ring containing 1 to 4 heteroatoms selected from oxygen, nitrogen or sulphur;

R² and R³ are independently hydrogen, halogen, C₁₋₆-alkyl, C₃₋₆-cycloalkyl, C₃₋₆-cycloalkenyl, C₁₋₆-alkoxy, hydroxyC₁₋₆-alkyl, C₁₋₆-alkyl, OC₁₋₆-alkyl, acyl, aryl, acyloxy, hydroxy, nitro, trifluoromethyl, cyano, CO₂R¹⁰, CONR¹⁰R¹¹, NR¹⁰R¹¹ where R¹⁰ and R¹¹ are independently hydrogen or C₁₋₆-alkyl;

R⁴ is hydrogen or C₁₋₆-alkyl;

R⁵ and R⁶ are independently hydrogen or C₁₋₆-alkyl;

A is (CR¹³R¹⁴) q where q is 2, 3 or 4 and R¹³ and R¹⁴ are independently hydrogen or C₁₋₆-alkyl or A is (CR¹³R¹⁴)ᵣ-D where r is 0, 1, 2 or 3 and D is oxygen, sulphur or CR¹³═CR¹⁴.

B is oxygen, CR¹⁵R¹⁶ or NR¹⁷ where R¹⁵, R¹⁶ and R¹⁷ are independently hydrogen or C₁₋₆alkyl or B is S(O) b where b is 0, 1 or 2;

m is 1, 2 or 3;

n is 1, 2 or 3;

or a salt or N-oxide thereof.

A preferred compound of formula S is 1′-methyl-5-[2′-methyl-4′-(5-methyl-1,2,4-oxadiazol-3-yl)biphenyl-4-yl]carbonyl]-2,3,6,7-tetrahydrospiro[furo[2,3-]indole-3 ,4′-piperidine.

In preferred embodiments, a compound having both 5-HT_{1A} and 5-HT_{1B} antagonist activities has a structure represented by formulas T, U, V or W below.

Formula T, disclosed in WO 98/14433 and U.S. Pat. No. 6,472,388, incorporated herein by reference in their entireties, describes compounds of the form 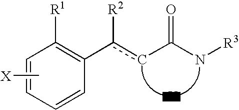
wherein for Formula T

R¹ is a member selected from the group consisting of G¹, G², G³, G⁴, G⁵, G⁶ and G⁷,

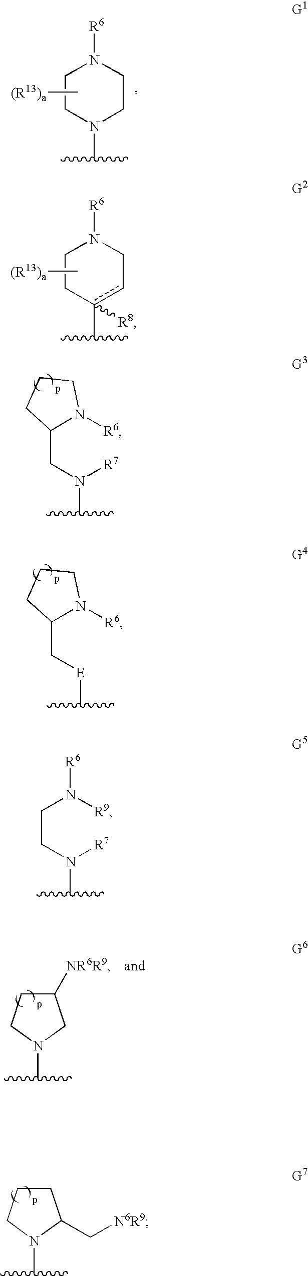

a is an integer from zero to eight;

each R¹³ is, independently, C₁₋₄-alkyl or a C₁₋₄-methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;

E is oxygen, sulfur, SO or SO₂;

X is hydrogen, chloro, fluoro, bromo, iodo, cyano, C₁₋₆-alkyl, hydroxy, trifluoromethyl, C₁₋₆-alkoxy, —SOₜC₁₋₆-alkyl wherein t is zero one or two, —CO₂R¹⁰ or —CONR¹¹R¹²;

Y is an optionally substituted C₁₋₄-heteroalkyl bridge that, together with the atoms to which it is attached, forms a five to seven membered heterocycle containing two to four heteroatoms selected from the group consisting of 1,3-oxazolidin-4-on-5-yl, 1,3-oxazolidin-2,4-dion-5-yl, 4,5-dihydro-1,2-oxazolidin-3-on-4-yl, 1,3-thiazolidin-4-on-5-yl, 1,3-thiazolidin-2,4-dion-5-yl, 1,3-pyrazolidin-4-on-5-yl, 1,3-imidazolidin-2,4-dion-5-1,2-pyrazolidin-3-on-4-yl, 1,2-thiazolidin-1,1,3-trion-4-yl, 1,2-thiazolidin-3-on-4-yl, tetrahydro-1,2-oxazin-3-on-4-yl, tetrahydro-1,3-oxazin-4-on-5-yl, tetrahydro-1,3-oxazin-2,4-dion-5-yl, morpholin-3-on-2-yl, morpholin-3,5-dion-2-yl, 2,3-dihydro-1,4-oxazin-3-on-2-yl, tetrahydro-1,3-thiazin-4-on-5-yl, tetrahydro-1,3-thiazin-2,4-dion-5-yl, tetrahydro-1,2-thiazin-3-on-4-yl, thiomorpholin-3-on-2-yl, thiomorpholin-3,5-dion-2-yl, 2,3-dihydro-1,4-thiazin-3-on-2-yl, hexahydro-1,2-diazin-3-on-4-yl, 4,5-dihydro-2H-pyridazin-3-on-4-yl, hexahydro-1,3-diazin-4-on-5-yl, hexahydro-1,3-diazin-2,4-dion-5-yl, piperazin-2-on-3-yl, piperazin-2,6-dion-3-yl, tetrahydro-1,3,4-thiadiazin-5-on-6-yl, 5,6-dihydro-1,3,4-thiadiazin-5-on-6-yl, 1,3,4-oxadiazin-5-on-6-yl, 5,6-dihydro-1,2,4-oxadiazin-5-on-6-yl, tetrahydro-1,2,4-oxadiazin-5-on-6-yl, 1,2,4-triazin-5-on-6-yl, tetrahydro-1,2,4-oxadiazin-5-on-6-yl, 5,6-dihydro-1,2,4-oxadiazin-5-on-6-yl, 1,2,4-oxadiazin-3,5-dion-6-yl, 1,2,4-trazin-6-on-5-yl, hexahydro-1,2-oxazepin-3-on-2-yl, hexahydro-1,3-oxazepin4-on-5-yl, hexahydro-1,4-oxazepin-3-on-2-yl, hexahydro-1,4-oxazepin-3,5-dion-2-yl, hexahydro-1,4-oxazepin-3,5-dion-6-yl, 2,3,5,6-tetrahydro-1-4-oxazepin-5,7-dion-4-yl, hexahydro-1,4-oxazepin-5-on-6-yl, hexahydro-1,3-oxazepin-2,4-dion-5-yl, hexahydro-1,2-thiazepin-3-on-4-yl, hexahydro-1,4-thiazepin-3-on-2-yl, 2,3,4,5-tetrahydro-1,4-thiazepin-3-on-2-yl, hexahydro-1,4-thiazepin-3,5-dion-2-yl, hexahydro-1,4-thiazepin-3,5-dion-6-yl, 2,3,6,7-tetrahydro-1,4-thiazepin-5-on-6-yl, 6,7-dihydro-1,4-thiazepin-5-on-6-yl, hexahydro-1,3-thiazepin-2,4-dion-5-yl, hexahydro-1,2-diazepin-3-on-4-yl, hexahydro-1,3-diazepin-2,4-dion-5-yl, hexahydro-1,4diazepin-2-on-3-yl, hexahydro-1,4-diazepin-5-on-6-yl, hexahydro-1,4diazepin-5,7-dion-6-yl, hexahydro-1,3,5-thiadiazepin-3-on-7-yl, 4,5,6,7-tetrahydro-1,3,5-thiadiazepin-6-on-7-yl, and 2,3,5,6-tetrahydro-1,2,4-triazepin-3,5-dion-7-yl;

wherein the substituents on any of the carbon atoms capable of supporting an additional bond, of said C,₄-heteroalkyl bridge, are chloro, fluoro, C₁₋₆-alkyl, C₁₋₆-alkoxy, trifluoromethyl or cyano; wherein the substituents on any of the nitrogen atoms capable of supporting an additional bond, of said C₁₋₄-heteroalkyl bridge, are C₁₋₆-alkyl or trifluoromethyl;

R² is hydrogen, C₁₋₄-alkyl, phenyl or naphthyl, wherein said phenyl or naphthyl may optionally be substituted with one or more substituents independently selected from chloro, fluoro, bromo, iodo, C₁₋₆-alkyl, C₁₋₆-alkoxy, trifluoromethyl, cyano and —SOₖC₁₋₆-alkyl wherein k is zero, one or two;

R³ is —(CH₂)ₘB, wherein m is zero, one, two or three and B is hydrogen, phenyl, naphthyl or a 5 or 6 membered heteroaryl group containing from one to four heteroatoms in the ring, and wherein each of the foregoing phenyl, naphthyl and heteroaryl groups may optionally be substituted with one or more substituents independently selected from chloro, fluoro, bromo, iodo, C₁₋₆-alkyl, C₁₋₆-alkoxy, C₁₋₆-alkoxyC₁₋₆-alkyl, trifluoromethyl, trifluoromethoxy, cyano, hydroxy, —COOH and —SOₙC₁₋₆-alkyl wherein n is zero, one or two;

R⁶ is selected from the group consisting of hydrogen, C₁₋₆-alkyl optionally substituted with C₁₋₆-alkoxy or one to three fluorine atoms, or (C₁₋₄-alkyl)aryl wherein the aryl moiety is phenyl, naphthyl, or heteroaryl—(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties may optionally be substituted with one or more substituents independently selected from the group consisting of chloro, fluoro, bromo, iodo, C₁₋₆-alkyl, C₁₋₆-alkoxy, trifluoromethyl, cyano and —SO_{g}C₁₋₆-alkyl, wherein g is zero, one or two;

R⁷ is selected from the group consisting of hydrogen, C₁₋₆-alkyl, (C₁₋₄-alkyl)aryl wherein the aryl moiety is phenyl, naphthyl, or heteroaryl—(CH₂)ᵣ—, wherein the heteroaryl moiety is selected from the group consisting of pyridyl, pyrimidyl, benzoxazolyl. benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, C₁₋₆-alkyl, C₁₋₆-alkoxy, trifluoromethyl, —C(O)—C₁₋₆-alkyl, cyano and —SOⱼC₁₋₆-alkyl, wherein j is zero, one or two;

or R⁶ and R⁷ taken together form a 2 to 4 carbon chain;

R⁸ is hydrogen or C₁₋₃-alkyl;

R⁹ is hydrogen or C₁₋₆-alkyl;

or R⁶ and R⁹, together with the nitrogen atom to which they are attached, form a 5- to 7-membered heteroalkyl ring that may contain from zero to four heteroatoms selected from nitrogen, sulfur and oxygen;

p is one, two, or three;

each of R¹⁰, R¹¹ and R¹² is selected, independently, from the radicals set forth in the definition of R²; or R¹¹ and R¹², together with the nitrogen to which they are attached, form a 5- to 7-membered heteroalkyl ring that may contain from zero to four heteroatoms selected from nitrogen, sulfur and oxygen; and

the broken lines indicate optional double bonds, with the proviso that when the broken line in G² is a double bond that R⁸ is absent;

or a pharmaceutically acceptable salt, enantiomer, diastereomer, N-oxide, crystalline form, hydrate, solvate, active metabolite or prodrug thereof.

A preferred compound of formula T is (Z)-4-(3,4-dichlorophenyl)-2-[2-(4-methylpiperazin-1-yl)benzylidene]thiomorpholin-3-one (elzasonan).

Formula U, disclosed in U.S. Pat. No. 6,222,034, incorporated herein by reference in its entirety, describes compounds of the form

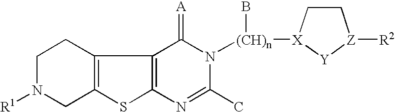

wherein for formula U

R¹ is hydrogen, C₁₋₄-alkyl, acetyl or benzoyl, a phenylalkyl C₁₋₄ radical, wherein the aromatic ring is unsubstituted or substituted by halogen, C₁₋₄-alkyl, trifluoromethyl, hydroxyl, C₁₋₄-alkoxy, amino, cyano or nitro groups, a naphthylalkyl C₁₋₃-radical, a phenylalkanone C₂₋₃-radical or a phenylcarbamoylalkyl C₂ radical, wherein the phenyl ring is unsubstituted or substituted by halogen,

R² is phenyl, pyridyl, pyrimidyl or pyrazinyl, each of which is unsubstituted or carries substituents selected from the group consistinrg of:

(i) one to three of the following: halogen, C₁₋₄-alkyl, trifluoromethyl, trifluoromethoxy, hydroxyl, C₁₋₄-alkoxy, amino, monomethylamino, dimethylamino, cyano and nitro, and

(ii) one phenyl-C₁₋₂-alkyl or phenyl-C₁₋₂-alkoxy, wherein the phenyl ring is unsubstituted or substituted by halogen, methyl, trilfuoromethyl or methoxy, or

is one of the foregoing unsubstituted or substituted phenyl, pyridyl, pyrimidyl or pyrazinyl radicals wherein two adjacent ring carbon atoms are bridged to form a benzo-fused or a pyridino-fused bicyclic wherein the bridging moiety is unsubstituted or substituted by one or two substituents selected from the group consisting of: halogen, C₁₋₄-alkyl, hydroxyl, trifluoromethyl, C₁₋₄-alkoxy, amino, cyano and nitro, or

is one of the foregoing unsubstituted or substituted phenyl, pyridyl, pyrimidyl or pyrazinyl radicals wherein two adjacent ring carbon atoms are bridged to form a 5- or 6-membered ring consisting of carbon ring members or carbon ring members and one or two oxygen atoms as ring members,

A is NH or an oxygen atom,

B is hydrogen or methyl,

C is hydrogen, methyl or hydroxyl,

X is a nitrogen atom,

Y is CH₂, CH₂—CH₂, CH₂—CH₂—CH₂ or CH₂—CH,

Z is a nitrogen atom, carbon atom or CH, wherein the linkage between Y and Z is a single or a double bond, and

n is 2, 3 or 4,

or a pharmaceutically acceptable salt, enantiomer, diastereomer, N-oxide, crystalline form, hydrate, solvate, active metabolite or prodrug thereof.

Formula V, disclosed in U.S. Pat. No. 6,355,647, incorporated herein by reference in its entirety, describes compounds of the form

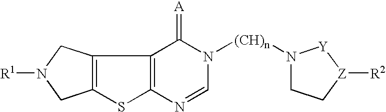

wherein for Formula V

R¹ is a hydrogen atom, a C₁₋₄-alkyl group, an acetyl group, a C₁₋₃-alkyl carboxylate radical, or is a phenyl-C₁₋₄-alkyl radical where the aromatic ring is unsubstituted or substituted by halogen, C₁₋₄-alkyl, trifluoromethyl, hydroxyl, C₁₋₄-alkoxy, amino, cyano or nitro groups,

R² is a phenyl, pyridyl, pyrimidinyl or pyrazinyl group which is unsubstituted or mono- or disubstituted by halogen atoms, C₁₋₄-alkyl, trifluoromethyl, trifluoromethoxy, hydroxyl, C₁₋₄-alkoxy, amino, monomethylamino, dimethylamino, cyano or nitro groups, and may be fused to a benzene nucleus which may be mono- or disubstituted by halogen atoms, C₁₋₄-alkyl, hydroxyl, trifluoromethyl, C₁₋₄-alkoxy, amino, cyano or nitro groups and may contain 1 nitrogen atom, or to a 5- or 6-membered ring which may contain 1-2 oxygen atoms,

A is NH or an oxygen atom,

Y is CH₂, CH₂—CH₂, CH₂—CH₂—CH₂ or CH₂—CH,

Z is a nitrogen atom, carbon atom or CH, where the linkage between Y and Z may also be a double bond, and

n is 2, 3 or 4,

or a pharmaceutically acceptable salt, enantiomer, diastereomer, N-oxide, crystalline form, hydrate, solvate, active metabolite or prodrug thereof.

Formula W, disclosed in U.S. Pat. No. 6,414,157, incorporated herein by reference in its entirety, describes compounds of the form

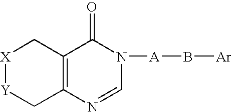

wherein for Formula W

one of the two radicals X and Y is CH₂ and the other is NR¹,

R¹ is hydrogen, C₁₋₈-alkyl, CO—C₁₋₄-alkyl, CO₂tBu, CO-aryl or phenylalkyl C₁₋₄-radical which in turn may be substituted on the aromatic system by F, Cl, Br, I, C₁₋₄-alkyl, C₁₋₄-alkoxy, trifluoromethyl, hydroxyl, amino, cyano or nitro,

A is C₁₋₁₀-alkylene or C₂₋₁₀-alkylene which comprises at least one group Z which is selected from O, S, NR², cyclopropyl, CO₂, CHOH, or a double or triple bond,

R² is hydrogen and C₁₋₄-alkyl,

B is 1,4-piperidinylene, 1,2,3,6-tetrahydro-1,4-pyridinylene, 1,4-piperazinylene or the corresponding cyclic compounds enlarged by one methylene group, with the linkage to A being via an N atom of B, and

Ar is phenyl which is unsubstituted or substituted by C₁₋₆-alkyl, O—C₁₋₈-alkyl, F, Cl, Br, I, trifluoromethyl, NR², CO₂R², cyano or phenyl, or is tetratinyl, indanyl, fused aromatic systems (e.g., naphthalene) which is unsubstituted or substituted by C₁₋₄-alkyl or O—C₁₋₄-alkyl, anthracene or 5- or 6-membered aromatic heterocycles having 1 or 2 heteroatoms which are selected, independently of one another, from O and N, which may be fused to other aromatic radicals,

or a pharmaceutically acceptable salt, enantiomer, diastereomer, N-oxide, crystalline form, hydrate, solvate, active metabolite or prodrug thereof.

### BRIEF DESCRIPTION OF DRAWINGS

FIG. 1 depicts the effect of co-administration of N-[2-[4-(2-methoxyphenyl)piperazin-1-yl]ethyl]-N-(2-pyridyl)cyclohexanecarboxamide with 3-[3-(dimethylamino)propyl]-4-hydroxy-N-[4-(4-pyridinyl)phenyl]benzamide in anaesthetized rats. Data represent the disappearance time (min) of bladder contractions in rats treated with N-[2-[4-(2-methoxyphenyl)piperazin-1-yl] ethyl]-N-(2-pyridyl)cyclohexanecarboxamide hydrochloride (WAY 100635) alone (0.01 mg/kg—open bar), 3-[3-(dimethylamino)propyl]-4-hydroxy-N-[4-(4-pyridinyl)phenyl]benzamide dihydrochloride (GR55562) alone (3 mg/kg—dashed bar) or the combination of N-[2-[4-(2-methoxyphenyl)piperazin-1-yl]ethyl]-N-(2-pyridyl)cyclohexanecarboxamide hydrochloride (0.01 mg/kg) and 3-[3-(dimethylamino)propyl]-4-hydroxy-N-[4-(4-pyridinyl)phenyl]benzamide dihydrochloride (3 mg/kg) (solid bar). The disappearance time of contractions induced by the association was significantly (p<0.01) different from that induced by administration of the single compounds alone.

FIG. 2 depicts the effect of co-administration of 1-[N-(2-nitrophenyl)-N-cyclohexylcarbonyl-2-aminoethyl]-4-(2-methoxyphenyl)piperazine with 3-[3-(dimethylamino)propyl]-4-hydroxy-N-[4-(4-pyridinyl)phenyl]benzamide in anaesthetized rats. Data represent the disappearance time (min) of bladder contractions in rats treated with 1-[N-(2-nitrophenyl)-N-cyclohexylcarbonyl-2-aminoethyl]-4-(2-methoxyphenyl)piperazine mesylate (REC 15/3079) alone (0.03 mg/kg—open bar), 3-[3-(dimethylamino)propyl]-4-hydroxy-N-[4-(4-pyridinyl)phenyl]benzamide dihydrochloride (GR 55562) alone (3 mg/kg—dashed bar) and that of the co-administration of 1-[N-(2-nitrophenyl)-N-cyclohexylcarbonyl-2-aminoethyl]-4-(2-methoxyphenyl)piperazine mesylate (0.03 mg/kg) and 3-[3-(dimethylamino)propyl]-4-hydroxy-N-[4-(4-pyridinyl)phenyl]benzamide dihydrochloride (3 mg/kg) (solid bar). The disappearance time induced by the association was significantly (p<0.01) different from that induced by administration of the single compounds alone.

FIG. 3 depicts the effect of co-administration of 2-[4-[4-(7-chloro-2,3-dihydro-1,4-benzodioxin-5-yl)piperazin-1-yl]butyl]-1,2-benzisothiazol-3(2H)-one 1,1-dioxide with 3-[3-(dimethylamino)propyl]-4-hydroxy-N-[4-(4-pyridinyl)phenyl]benzamide in anaesthetized rats. Data represent the disappearance time (min) of bladder contractions in rats treated with 2-[4-[4-(7-chloro-2,3-dihydro-1,4-benzodioxin-5-yl)piperazin-1-yl]butyl]-1,2-benzisothiazol-3(2H)-one 1,1-dioxide dihydrochloride (Rec 0/031 1-DU 125530) alone (0.01 mg/kg—open bar), 3-[3-(dimethylamino)propyl]-4-hydroxy-N-[4-(4-pyridinyl)phenyl]benzamide dihydrochloride (GR55562) alone (3 mg/kg—dashedbar) and that of the co-administration of 2-[4-[4-(7-chloro-2,3-dihydro-1,4-benzodioxin-5-yl)piperazin-1-yl]butyl]-1,2-benzisothiazol-3(2H)-one 1,1-dioxide dihydrochloride (0.01 mg/kg) and 3-[3-(dimethylamino)propyl]-4-hydroxy-N-[4-(4-pyridinyl)phenyl]benzamide dihydrochloride (3 mg/kg) (solid bar). The disappearance time induced by the association was significantly (p<0.01) different from that induced by administration of the single compounds alone.

FIG. 4 depicts the effect of co-administration of 1-[3-hydroxy-3,3 bis-(2-pyridyl)propyl]-4-(4-indolyl)piperazine with 3-[3-(dimethylamino)propyl]-4-hydroxy-N-[4-(4-pyridinyl)phenyl]benzamide in anaesthetized rats. Data represent the disappearance time (min) of bladder contractions in rats treated with 1-[3-hydroxy-3,3 bis-(2-pyridyl)propyl]-4-(4-indolyl)piperazine (Rec 27/0206) alone (0.1 mg/kg—open bar), 3-[3-(dimethylamino)propyl]-4-hydroxy-N-[4-(4-pyridinyl)phenyl]benzamide dihydrochloride (GR 55562) alone (3 mg/kg—dashed bar) and that of the co-administration of 1-[3-hydroxy-3,3 bis-(2-pyridyl)propyl]-4-(4-indolyl)piperazine (0.1 mg/kg) and 3-[3-(dimethylamino)propyl]-4-hydroxy-N-[4-(4-pyridinyl)phenyl]benzamide dihydrochloride (3 mg/kg) (solid bar). The disappearance time induced by the association was significantly (p<0.05) different from that induced by administration of the single compounds alone.

FIG. 5 depicts the effect of co-administration of 2-[4-[4-(7-chloro-2,3-dihydro-1,4-benzodioxin-5-yl)piperazin-1-yl]butyl]-1,2-benzisothiazol-3 (2H)-one 1,1-dioxide with 1′-methyl-5-[(2′-methyl-4′-(5-methyl-1,2,4-oxadiazol-3-yl) biphenyl-4-yl]carbonyl]-2,3,6,7-tetrahydrospiro[furo[2,3-f]indole-3,4′-piperidine in anaesthetized rats. Data represent the disappearance time (min) of bladder contractions in rats treated with 2-[4-[4-(7-chloro-2,3-dihydro-1,4-benzodioxin-5-yl)piperazin-1-yl]butyl]-1,2-benzisothiazol-3(2H)-one 1,1-dioxide dihydrochloride (Rec 0/031 I-DU 125530) alone (0.01 mg/kg—open bar), 1′-methyl-5-[(2′-methyl-4′-(5-methyl-1,2,4-oxadiazol-3-yl) biphenyl-4-yl]carbonyl]-2,3,6,7-tetrahydrospiro[furo[2,3-f]indole-3,4′-piperidine hydrochloride (SB 224289) alone (1 mg/kg—dashed bar) and that of the co-administration of 2-[4-[4-(7-chloro-2,3-dihydro-1,4-benzodioxin-5-yl)piperazin-1-yl]butyl]-1,2-benzisothiazol-3(2H)-one 1,1-dioxide dihydrochloride (0.01 mg/kg) and 1′-methyl-5-[(2′-methyl-4′-(5-methyl-1,2,4-oxadiazol-3-yl) biphenyl-4-yl]carbonyl]-2,3,6,7-tetrahydrospiro[furo[2,3-f]indole-3,4′-piperidine hydrochloride (1 mg/kg) (solid bar). The disappearance time induced by the association was significantly (p<0.01) different from that induced by administration of the single compounds alone.

FIG. 6 depicts the effect of co-administration of 1-[3-hydroxy-3,3 bis-(2-pyridyl)propyl]-4-(4-indolyl)piperazine with 1′-methyl-5-[(2′-methyl-4′-(5-methyl-1,2,4-oxadiazol-3-yl) biphenyl-4-yl]carbonyl]-2,3,6,7-tetrahydrospiro[furo[2,3-f]indole-3,4′ -piperidine in anaesthetized rats. Data represent the disappearance time (min) of bladder contractions in rats treated with 1-[3-hydroxy-3,3 bis-(2-pyridyl)propyl]-4-(4-indolyl)piperazine (Rec 27/0206) alone (0.1 mg/kg—open bar), 1 ′-methyl-5-[(2′-methyl-4′-(5-methyl-1,2,4-oxadiazol-3-yl) biphenyl-4-yl]carbonyl]-2,3,6,7-tetrahydrospiro[furo[2,3-f]indole-3,4-piperidine hydrochloride (SB 224289) alone (1 mg/kg—dashed bar) and that of the co-administration of 1-[3-hydroxy-3,3 bis-(2-pyridyl)propyl]-4-(4-indolyl)piperazine (0.1 mg/kg) and 1′-methyl-5-[(2′-methyl-4′-(5-methyl-1,2,4-oxadiazo]-3-yl) biphenyl-4-yl]carbonyl]-2,3,6,7-tetrahydrospiro]furo[2,3-f]indole-3,4′-piperidine hydrochloride (1 mg/kg) (solid bar). The disappearance time induced by the association was significantly (p<0.05) different from that induced by administration of the single compounds alone.

FIG. 7 depicts the effect of co-administration of 1-[3-hydroxy-3,3 bis-(2-pyridyl)propyl]-4-(4-indolyl)piperazine with N-[3-[2-(dimethylamino)ethoxy]-4-methoxyphenyl-2′-methyl-4′-(5-methyl-1,2,4-oxadiazo]-3-yl)-[1,1′-biphenyl]-4-carboxamide in anaesthetized rats. Data represent the disappearance time (min) of bladder contractions in rats treated with 1-[3-hydroxy-3,3 bis-(2-pyridyl)propyl]-4-(4-indolyl)piperazine (Rec 27/0206) alone (0.1 mg/kg—open bar), N-[3-[2-(dimethylamino)ethoxy]-4-methoxyphenyl-2′-methyl-4′-(5-methyl-1,2,4-oxadiazo]-3-yl)-[1,1′-biphenyl]-4-carboxamide hydrochloride (SB 216641) alone (0.3 mg/kg—dashed bar) and that of the co-administration of 1-[3-hydroxy-3,3 bis-(2-pyridyl)propyl]-4-(4-indolyl)piperazine (0.1 mg/kg) and N-[3-[2-(dimethylamino)ethoxy]-4-methoxyphenyl-2′-methyl-4′-(5-methyl-1,2,4-oxadiazo]-3-yl)-[1,1′-biphenyl]-4-carboxamide hydrochloride (0.3 mg/kg) (solid bar). The disappearance time induced by the association was significantly (p<0.01) different from that induced by administration of the single compounds alone.

FIG. 8 depicts the effect of co-administration of 1-cyclohexyl-4-[4-(2-methoxyphenyl)-piperazin-1-yl]-2-pyridin-2-yl-butan-1-one with N-[3-[2-(dimethylamino)ethoxy]-4-methoxyphenyl-2′-methyl-4′-(5-methyl-1,2,4-oxadiazo]-3-yl)-[1,1′-biphenyl]-4-carboxamide in anaesthetized rats. Data represent the disappearance time (min) of bladder contractions in rats treated with 1-cyclohexyl-4-[4-(2-methoxyphenyl)-piperazin-1-yl]-2-pyridin-2-yl-butan-1-one (Rec 0/0277) alone (0.01 mg/kg—open bar), N-[3-[2-(dimethylamino)ethoxy]-4-methoxyphenyl-2′-methyl-4′-(5-methyl-1,2,4-oxadiazol-3-yl)-[1,1′-biphenyl]-4-carboxamide hydrochloride ( SB 216641) alone (0.3 mg/kg—dashed bar) and that of the co-administration of 1-cyclohexyl-4-[4-(2-methoxyphenyl)-piperazin-1-yl]-2-pyridin-2-yl-butan-1-one (0.01 mg/kg) and N-[3-[2-(dimethylamino)ethoxy]-4-methoxyphenyl-2′-methyl-4′-(5-methyl-1,2,4-oxadiazol-3-yl)-[1,1′-biphenyl]-4-carboxamide hydrochloride (0.3 mg/kg) (solid bar). The disappearance time induced by the association was significantly (p<0.01) different from that induced by administration of the single compounds alone.

FIG. 9 depicts the effect of the i.v. administration of N-[2-[4-(2-methoxyphenyl)piperazin-1-yl] ethyl]-N-(2-pyridyl)cyclohexanecarboxamide hydrochloride (WAY 100635) 0.01 mg/kg, 3-[3-(dimethylamino)propyl]-4-hydroxy-N-[4-(4-pyridinyl)phenyl]benzamide dihydrochloride (GR 55562) 3 mg/kg and of the co-administration of N-[2-[4-(2-methoxyphenyl)piperazin-1-yl]ethyl]-N-(2-pyridyl)cyclohexanecarboxamide hydrochloride (0.01 mg/kg) and 3-[3-(dimethylamino)propyl]-4-hydroxy-N-[4-(4-pyridinyl)phenyl]benzamide dihydrochloride (3.0 mg/kg) on BVC in conscious rats. Data represent the ΔAUC values (practical AUC-theoretical AUC) calculated during the 5 hr of recording period. The increase of BVC induced by the combination was significantly (p<0.05) different from that of all the other treatments (ONE WAY ANOVA and Tukey's test).

### DETAILED DESCRIPTION OF THE INVENTION

All patents, patent applications and literature references cited in the description are herein incorporated by reference in their entirety. In′ the case of inconsistencies, the present disclosure, including definitions, will prevail. The present invention is concerned with methods of using a combination of antagonists and/or inverse agonists of 5-HT_{1A} and 5-HT_{1B} receptors for treating neuromuscular dysfunction of the lower urinary tract. The antagonists and/or inverse agonists are preferably antagonists of human 5-HT_{1A} and/or 5-HT_{1B} receptors.

The invention is based on the findings that treatment with (1) a compound endowed with antagonistic activity at 5-HT_{1A} receptors in combination with a compound endowed with antagonistic activity at 5-HT_{1B} receptors, or (2) a compound simultaneously endowed with antagonistic activity at the 5-HT_{1A} and 5-HT_{1B} receptors, gave unexpectedly superior results for treatment of neuromuscular dysfunction of the lower urinary tract.

The invention also includes metabolites having the same type of activity, hereinafter referred to as active metabolites.

The present invention also contemplates prodrugs which are metabolised in the body to generate compounds having antagonistic activity at a 5-HT_{1A} or 5-HT_{1B} receptor, or at both the 5-HT_{1A} and 5-HT_{1B} receptors.

In another embodiment, the present invention provides pharmaceutical compositions comprising compounds having the same type of activity, enantiomers, diastereomers, N-oxides, crystalline forms, hydrates, solvates or pharmaceutically acceptable salts of such compounds, in admixture with pharmaceutically acceptable diluents or carriers such as those disclosed.

In yet another embodiment, the present invention provides the use of an effective amount of a compound endowed with antagonistic activity at 5-HT_{1A} receptors in combination with an effective amount of a compound endowed with antagonistic activity at 5-HT_{1B} receptors, or an effective amount of a compound simultaneously endowed with antagonistic activity at the 5-HT_{1A} and 5-HT_{1B} receptors for reducing the frequency of bladder contractions due to bladder distension by administering said combination of compounds or said compound to a mammal, including a human, in need of such treatment.

In yet another embodiment, the present invention provides the use of an effective amount of a compound endowed with antagonistic activity at 5-HT_{1A} receptors in combination with an effective amount of a compound endowed with antagonistic activity at 5-HT_{1B} receptors, or an effective amount of a compound simultaneously endowed with antagonistic activity at the 5-HT_{1A} and 5-HT_{1B} receptors for increasing urinary bladder capacity by administering said combination of compounds or said compound to a mammal, including a human, in need of such treatment.

In yet another embodiment, the present invention provides the use of an effective amount of a compound endowed with antagonistic activity at 5-HT_{1A} receptors in combination with an effective amount of a compound endowed with antagonistic activity at 5-HT_{1B} receptors, or an effective amount of a compound simultaneously endowed with antagonistic activity at the 5-HT_{1A} and 5-HT_{1B} receptors in an amount effective for treating disorders of the urinary tract in a patient in need of such treatment to ameliorate at least one condition among urinary urgency, overactive bladder, increased urinary frequency, decreased urinary compliance (decreased bladder storage capacity), cystitis (including interstitial cystitis), incontinence, urine leakage, enuresis, dysuria, urinary hesitancy and difficulty in emptying the bladder.

For treating the above disorders, the compounds of the invention may be administered in combination with known antimuscarinic drugs such as, without limitation, oxybutynin, tolterodine, darifenacin and temiverine. Analogously, the compounds of the invention may be associated to x I-adrenergic antagonists such as, without limitation, prazosin, doxazosin, terazosin, alfuzosin and tamsulosin, for the therapy of lower urinary tract symptoms, whether or not these are associated with BPH.

Treatment may be effected by delivering to the environment of 5-HT_{1A} and 5-HT_{1B} serotonergic receptor, for example, and without limitation, to the extracellular medium, or by systemically or locally administering to a mammal possessing such receptor, an amount of a compound of the invention effective to increase the duration of bladder quiescence with no contractions. The present invention refers to a method of administering a compound or compounds of the formulas set forth above.

Combination therapy with 5-HT_{1A} and 5-HT_{1B} antagonists may further include an α1-adrenergic antagonist, for the therapy of lower urinary tract symptoms, whether or not these are associated with BPH. Preferred α1-adrenergic antagonists suitable for administration in combination with a selective 5-HT_{1A} and/or 5-HT_{1B} antagonist are, for example, and without limitation, prazosin, doxazosin, terazosin, alfuzosin, and tamsulosin.

Additional ccl-adrenergic antagonists suitable for administration in combination with 5-HT_{1A} and 5-HT_{1B} antagonist are described in U.S. Pat. Nos. 5,990,114; 6,306,861; 6,365,591; 6,387,909; and 6,403,594, incorporated herein by reference in their entireties.

Examples of 5-HT_{1A} antagonists are found in Leonardi et al., J. Pharmacol. Exp.

Ther. 299: 1027-1037, 2001(e.g., Rec 15/3079), U.S. Pat. Nos. 6,071,920, 6,399,614, 6,271,234, 5,990,114, incorporated herein by reference in their entirety. Other phenylpiperazine derivatives are described in WO 99/06383 and pending U.S. patent applications Ser. Nos. 10/266,088 and 10/266,104 filed on Oct. 7, 2002. Additional 5-HT_{1A} antagonists include 2-[4-[4-(7-chloro-2,3-dihydro-1,4-benzodioxin-5-yl)piperazin-1-yl]butyl]-1,2-benzisothiazol-3-(2H]-one-1,1 dioxide and related compounds described in U.S. Pat. No. 5,462,942 and robalzotan and related compounds described in WO 95/11891, incorporated herein by reference in their entireties.

Compounds having 5-HT_{1A} antagonist activity and α1 adrenergic receptor activity are described in U.S. Pat. Nos. 5,605,896, 5,474,994, and 5,403,842, 5,462,942, 6,127,357, incorporated herein by reference in their entireties. Preferred 5-HT_{1A} compounds of the invention are N-[2-[4-(2-methoxyphenyl)piperazin-1-yl]ethyl]-N-(2-pyridyl)cyclohexanecarboxamide, 2-[4-[4-(7-chloro-2,3-dihydro-1,4-benzodioxin-5-yl)piperazin-1-yl]butyl]-1,2-benzisothiazol-3-(2H]-one-1,1 dioxide, 1-[N-(2-nitrophenyl)-N-cyclohexylcarbonyl-2-aminoethyl]-4-(2-methoxyphenyl)piperazine, 1-[3-hydroxy-3,3 bis-(2-pyridyl)propyl]-4-(4-indolyl)piperazine.

Preferred 5HT_{1B} compounds of the invention are N-[3-[3-(dimethylamino)ethoxy]-4-methoxyphenyl-2′-methyl-4′-(5-methyl-1,2,4-oxadiazol-3-yl)-[ 1,1′-biphenyl]-4-carboxamide, 1′-methyl-5-[(2′-methyl-4′-(5-methyl-1,2,4-oxadiazol-3-yl) biphenyl-4-yl]carbonyl]-2,3,6,7-tetrahydrospiro[furo[2,3-f]indole-3,4′-piperidine, and 3-[3-dimethylamino)propyl]-4-hydroxy-N-[4-(4-pyridinyl)phenyl]benzamide. A preferred compound having both 5HT_{1A} and 5HT_{1B} activity is (Z)-4-(3,4-dichlorophenyl)-2-[2-(4-methylpiperazin-1-yl)benzylidene]thiomorpholin-3-one.

Pharmacological blocking of the 5-HT_{1A} and/or 5-HT_{1B} receptor leads to positive effects in the management of neuromuscular dysfunction of the lower urinary tract. An antagonist of the 5-HT_{1A} and/or 5-HT_{1B} receptor is a substance which diminishes or abolishes the effect of a ligand (agonist) which typically activates the 5-HT_{1A} and/or 5-HT_{1B} receptor. The antagonist may be, for example, a chemical antagonist, a pharmacokinetic antagonist, an antagonist by receptor block, a non-competitive antagonist or a physiological antagonist.

A chemical antagonist is a substance wherein the antagonist binds the ligand in solution so the effect of the ligand is lost. A pharmacokinetic antagonist is one which effectively reduces the concentration of the active ligand at its site of action, for example, by increasing the rate of metabolic degradation of the active ligand. Antagonism by receptor-block involves two important mechanisms: reversible competitive antagonism and irreversible, or non-equilibrium competitive antagonism. Reversible competitive antagonism occurs when the rate of dissociation of the antagonist molecules is sufficiently high such that, on addition of the ligand, displacement of chemical antagonist molecules from the receptors effectively occurs. Of course the ligand cannot displace a bound antagonist molecule, or vice versa. Irreversible or non-equilibrium competitive antagonism occurs when the antagonist dissociates very slowly, or not at all, from the receptor with the result that no change in the antagonist occupancy takes place when the ligand is applied. Thus, the antagonism is irreversible. Non-competitive antagonism describes the situation where the antagonist blocks at some point in the signal transduction pathway leading to the production of a response by the ligand.

Physiological antagonism is a term used loosely to describe the interaction of two substances whose opposing actions in the body tend to cancel each other out. An antagonist can also be a substance which diminishes or abolishes expression of functional 5-HT_{1A} and/or 5-HT_{1B} receptor. Thus an antagonist can be, for example, a substance which diminishes or abolishes expression of the gene encoding either the 5-HT_{1A} or 5-HT_{1B} receptor, diminishes or abolishes translation of either the 5-HT_{1A} or 5-HT_{1B} receptor RNA, diminishes or abolishes post-translational modification of either the 5-HT_{1A} or 5-HT_{1B} receptor protein or diminishes or abolishes the insertion of either the 5-HT_{1A} or 5-HT_{1B} receptor into the cell membrane.

An inverse agonist of either the 5-HT_{1A} or 5-HT_{1B} receptor is a substance which preferentially binds to the inactive state of the receptor (in contrast to the agonists that bind preferentially to the active state of the receptor), and therefore avoids the stimulation of the receptor by the agonist.

In general, the in vivo activity of inverse agonists is similar to that of antagonists and for the sake of clarity inverse agonists will be defmed as antagonists in the present application.

5-HT_{1A} or 5-HT_{1B} antagonists have the following properties.

(1) Significant 5-HT_{1A} or 5-HT_{1B} Antagonist Activity.

Useful compounds preferably exhibit antagonist potency (measured as IC₅₀ or Ki) between 1000 and 0.1 nM. Without limiting the present disclosure, as described in more detail below, potency may be measured by determining the antagonist activity of compounds in vivo or in vitro, including cell extracts or fractions of extracts. Inhibitory potency may also be determined using, as non-limiting examples, native or recombinant 5-HT_{1A} or 5-HT_{1B} receptors, that are expressed constitutively or that have been induced, and that have expressed in native or non-native species and/or cell types (Barnes N M and Sharp T. Neuropharmacology 38: 1083-1152, 1999).

Preferably, the compounds of the method of the present invention have a selectivity toward one or both of 5HT_{1A} and 5HT_{1B} receptors that is at least ten-fold compared to other 5HT receptor subtypes, e.g., 5HT₂, 5HT₃, 5HT₄.

The commonly used in vitro assays for assessing antagonist activity for 5-HT_{1A} or 5-HT_{1B} receptors are found in (Pauwels P J et al., Neuropharmacology 36: 499-512, 1997). In preferred embodiment, measurement of antagonist activity at either a 5-HT_{1A} or 5-HT_{1B} receptor is performed using one or more of the assays described in the examples set forth below. Using one or more of said assays, the antagonist activity at either a 5-HT_{1A} or 5-HT_{1B} receptor of a test compound can be measured, and the concentration inhibiting binding by 50% (IC₅₀) can be calculated using regression analysis, or equivalent computational methods that are well-known in the art (Tallarida et al., Manual of Pharmacologic Calculations. Springer-Verlag, pp. 10-12, 1981).

Once a compound is identified as a 5-HT_{1A} or 5-HT_{1B} antagonist, its pharmacological activity can be confirmed using one or more animal model systems for neuromuscular dysfunction of the lower urinary tract.

A useful animal model system for measuring such pharmacological activity is, without limitation, volume-induced rhythmic bladder voiding contractions in anesthetized rats. In this method, the urinary bladder is catheterized through the external urethra with a polyethylene tubing filled with physiological saline. The external urethra is then ligated and connected to a pressure recording device. The bladder is then filled with saline until reflex voiding contractions occur, after which the frequency of the voiding contractions is measured for 15 min. Test compounds are then administered intravenously and their effect evaluated for the following 60 min. This method is described in more detail in Example 3 below. This model has been validated by the use of different reference standards (Guarneri et al., Pharmacol. Res. 27:173-187, 1993).

Other animal models useful to assess activity of the selective 5-HT_{1A} or 5-HT_{1B} antagonists on the lower urinary tract are based on cystometric recording of bladder activity in conscious rats instrumented in order to measure bladder pressure during constant infusion of the bladder with saline or very diluted acetic acid. Velasco C. et al., J. Urol. 166: 1962-1968, 2001. These methods are widely used and accepted by researchers skilled in this field and foresee a period of infusion of about five hours after administration of test compounds with continuous monitoring of bladder performance and assessment of intervals between micturitions and peak micturition pressure.

A metabolite of a compound disclosed herein is a derivative of a compound which is formed when the compound is metabolised. The term “active metabolite” refers to a biologically active derivative of a compound which is formed when the compound is metabolised.

The term “metabolised” refers to the sum of the processes by which a particular substance is changed in the living body. In brief, all compounds present in the body are manipulated by enzymes within the body in order to derive energy and/or to remove them from the body. Specific enzymes produce specific structural alterations to the compound. For example, cytochrome P450 catalyses a variety of oxidative and reductive reactions while uridine diphosphate glucuronyltransferases catalyse the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996), pages 11-17.

Metabolites of the compounds disclosed herein can be identified either by administration of compounds to a host and analysis of tissue samples from the host, or by incubation of compounds with hepatic cells in vitro and analysis of the resulting compounds. Both methods are well known in the art.

A “prodrug” of a compound disclosed herein is an inactive substance that converts into an active form of the disclosed compounds in vivo when administered to a mammal.

Pharmaceutical Compositions

The invention provides pharmaceutical compositions comprising a compound endowed with antagonistic activity at 5-HT_{1A} receptors and a compound endowed with antagonistic activity at 5-HT_{1B} receptors or an enantiomer, etc. The invention also provides pharmaceutical compositions comprising at least one compound simultaneously endowed with antagonistic activity at the 5-HT_{1A} and 5-HT_{1B} receptors or an enantiomer, diastereomer, N-oxide, crystalline form, hydrate, solvate, active metabolite or pharmaceutically acceptable salt of the antagonists.

A pharmaceutical composition may also include optional additives, such as a pharmaceutically acceptable carrier or diluent, a flavouring, a sweetener, a preservative, a dye, a binder, a suspending agent, a dispersing agent, a colorant, a disintegrator, an excipient, a diluent, a lubricant, an absorption enhancer, a bactericide and the like, a stabiliser, a plasticizer, an edible oil, or any combination of two or more of said additives.

Suitable pharmaceutically acceptable carriers or diluents include, but are not limited to, ethanol, water, glycerol, aloe vera gel, allantoin, glycerine, vitamin-A and E oils, mineral oil, phosphate buffered saline, PPG2 myristyl propionate, magnesium carbonate, potassium phosphate, vegetable oil, animal oil and solketal.

Suitable binders include, but are not limited to, starch, gelatine, natural sugars such as glucose, sucrose and lactose, corn sweeteners, natural and synthetic gums such as acacia, tragacanth, vegetable gum, sodium alginate, carboxymethylcellulose, polyethylene glycol, waxes and the like.

Suitable disintegrators include, but are not limited to, starch such as corn starch, methyl cellulose, agar, bentonite, xanthan gum and the like.

Suitable lubricants include, but are not limited to, sodium oleate, sodium stearate, magnesium stearate, sodium benzoate, sodium acetate, sodium chloride and the like.

Suitable suspending agents include, but are not limited to, bentonite.

Suitable dispersing and suspending agents include, but are not limited to, synthetic and natural gums such as vegetable gum, tragacanth, acacia, alginate, dextran, sodium carboxymethylcellulose, methylcellulose, polyvinylpyrrolidone and gelatine.

Suitable edible oils include, but are not limited to, cottonseed oil, sesame oil, coconut oil and peanut oil.

Examples of additional additives include, but are not limited to, sorbitol, talc, stearic acid and dicalcium phosphate.

Unit Dosage Forms

The pharmaceutical composition may be formulated as unit dosage forms, such as tablets, pills, capsules, boluses, powders, granules, sterile parenteral solutions, sterile parenteral suspensions, sterile parenteral emulsions, elixirs, tinctures, metered aerosol or liquid sprays, drops, ampoules, autoinjector devices or suppositories. The unit dosage forms may be used for oral, parenteral, intranasal, sublingual or rectal administration, or for administration by inhalation or insufflation, transdermal patches, and a lyophilized composition. In general, any delivery of active ingredients that results in systemic availability of such ingredients can be used. Preferably the unit dosage form is an oral dosage form, most preferably a solid oral dosage; therefore the preferred dosage forms are tablets, pills and capsules. However, parenteral preparations are preferred too.

Solid unit dosage forms may be prepared by mixing the active agents of the present invention with a pharmaceutically acceptable carrier and any other desired additives as described above. The mixture is typically mixed until a homogeneous mixture of the active agents of the present invention is obtained and the carrier and any other desired additives are formed, i.e., the active agents are dispersed evenly throughout the composition. In this case, the composition can be formed as dry or moist granules.

Tablets or pills can be coated or otherwise prepared so as to form a unit dosage form that has delayed and/or sustained action, such as controlled release and delayed release unit dosage forms. For example, the tablet or pill can comprise an inner dosage and an outer dosage component, the latter being in the form of a layer or envelope over the former. The two components can be separated by an enteric layer which serves to resist disintegration in the stomach and permits the inner component to pass intact into the duodenum or to be delayed in release.

Biodegradable polymers for controlling the release of the active agents include, but are not limited to, polylactic acid, polyepsilon caprolactone, polyhydroxybutyric acid, polyorthoesters, polyacetals, polydihydropyrans, polycyanoacrylates and crosslinked or amphipathic block copolymers of hydrogels.

For liquid dosage forms, the active substances or their physiologically acceptable salts are dissolved, suspended or emulsified, optionally with the usually employed substances such as solubilizers, emulsifiers or other auxiliaries. Solvents for the active combinations and the corresponding physiologically acceptable salts can include water, physiological salt solutions or alcohols, e.g., ethanol, propanediol or glycerol. Additionally, sugar solutions such as glucose or mannitol solutions may be used. A mixture of the various solvents mentioned may be used in the present invention too.

A transdermal dosage form is contemplated by the present invention too. Transdermal forms may be a diffusion transdermal system (transdermal patch) using either a fluid reservoir or a drug-in-adhesive matrix system. Other transdermal dosage forms include, but are not limited to, topical gels, lotions, ointments, transmucosal systems and devices, and iontophoretic (electrical diffusion) delivery systems. Transdermal dosage forms may be used for delayed release and sustained release of the active agents of the present invention.

The pharmaceutical compositions and unit dosage forms of the present invention for parenteral administration, and in particular by injection, typically include a pharmaceutically acceptable carrier, as described above. A preferred liquid carrier is vegetable oil. Injection may be, for example, intravenous, epidural, intrathecal, intramuscular, intraluminal, intratracheal or subcutaneous.

The active agents can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

The active agents of the present invention may also be coupled with soluble polymers such as targetable drug carriers. Such polymers include, but are not limited to, polyvinylpyrrolidone, pyran copolymers, polyhydroxypropylmethacrylamidophenol, polyhydroxyethylaspartamidophenol, and polyethylenoxypolylysine substituted with palmitoyl residues.

Administration

The pharmaceutical composition or unit dosage forms of the present invention may be administered by a variety of routes, such as the oral and enteral, intravenous, intramuscular subcutaneous, transdermal, transmucosal (including rectal and buccal) and by inhalation routes. Preferably, the oral or transdermal route is used (i.e., with solid or liquid formulations or skin patches, respectively).

The pharmaceutical composition or unit dosage forms comprising an effective amount of the present invention may be administered to an animal, preferably a human, in need of treatment of neuromuscular dysfumction of the lower urinary tract described by E. J. McGuire in “Campbell's UROLOGY”, ₅th Ed. 616-638, 1986, W.B. Saunders Company, and patients affected by any physiological dysfunction related to impairment of 5-HT_{1A} and 5-HT_{1B} receptor function.

As used herein, the term “effective amount” refers to an amount that results in measurable amelioration of at least one symptom or parameter of a specific disorder. In a preferred embodiment, the compound treats disorders of the urinary tract, such as urinary urgency, overactive bladder, increased urinary frequency, reduced urinary compliance (reduced bladder storage capacity), cystitis (including interstitial cystitis), incontinence, urine leakage, enuresis, dysuria, urinary hesitancy and difficulty in emptying the bladder.

The pharmaceutical composition or unit dosage form of the present invention may be administered according to a dosage and administration regimen defined by routine testing in the light of the guidelines given above in order to obtain optimal activity while minimising toxicity or side effects for a particular patient. However, such fine tuning of the therapeutic regimen is routine in the light of the guidelines given herein.

The dosage of the active agents of the present invention may vary according to a variety of factors such as underlying disease conditions, the individual's condition, weight, sex and age, and the mode of administration. An effective amount for treating a disorder can easily be determined by empirical methods known to those of ordinary skill in the art, for example by establishing a matrix of dosages and frequencies of administration and comparing a group of experimental units or subjects at each point in the matrix. The exact amount to be administered to a patient will vary depending on the state and severity of the disorder and the physical condition of the patient. A measurable amelioration of any symptom or parameter can be determined by a person skilled in the art or reported by the patient to the physician. It will be understood that any clinically or statistically significant attenuation or amelioration of any symptom or parameter of urinary tract disorders is within the scope of the invention. Clinically significant attenuation or amelioration means perceptible to the patient and/or to the physician.

For example, a single patient may suffer from several symptoms of dysuria simultaneously, such as, for example, urgency and excessive frequency of urination or both, and these may be reduced using the methods of the present invention. In the case of incontinence, any reduction in the frequency or volume of unwanted passage of urine is considered a beneficial effect of the present method of treatment.

The amount of the agent to be administered can typically range between about 0.01 and about 25 mg/kg/day, preferably between about 0.1 and about 10 mg/kg/day and most preferably between 0.2 and about 5 mg/kg/day. It will be understood that the pharmaceutical formulations of the present invention need not necessarily contain the entire amount of the agent that is effective in treating the disorder, as such effective amounts can be reached by administration of a plurality of doses of such pharmaceutical formulations.

In a preferred embodiment of the present invention, the compounds are formulated in capsules or tablets, preferably containing 50 to 200 mg of the compounds of the invention, and are preferably administered to a patient at a total daily dose of 50 to 400 mg, preferably 150 to 250 mg and most preferably about 200 mg, for relief of urinary incontinence and dysfunctions under treatment with 5-HT_{1A} and/or 5HT_{1B} receptor ligand.

A pharmaceutical composition for parenteral administration contains from about 0.01% to about 100% by weight of the active agents of the present invention, based upon 100% weight of total pharmaceutical composition.

Generally, transdermal dosage forms contain from about 0.01% to about 100% by weight of the active agents versus 100% total weight of the dosage form.

The pharmaceutical composition or unit dosage form may be administered in a single daily dose, or the total daily dosage may be administered in divided doses. In addition, co-administration or sequential administration of another compound for the treatment of the disorder may be desirable. For example, the combinations of the invention may be administered in combination with known antimuscarinic drugs such as oxybutynin, tolterodine, darifenacin and temiverine. Analogously, the combinations of the invention may be associated to cc -adrenergic antagonists, such as prazosin, doxazosin, terazosin, alfuzosin and tamsulosin for the therapy of the lower urinary tract symptoms.

For combination treatment where the compounds are in separate dosage formulations, the compounds can be administered concurrently, or each can be administered at separate staggered times. For example, the compound of the invention may be administered in the morning and the antimuscarinic compound may be administered in the evening, or vice versa. Additional compounds may be administered at specific intervals too. The order of administration will depend upon a variety of factors including age, weight, sex and medical condition of the patient; the severity and aetiology of the disorders to be treated, the route of administration, the renal and hepatic function of the patient, the treatment history of the patient, and the responsiveness of the patient. Determination of the order of administration may be fine-tuned and such fine-tuning is routine in the light of the guidelines given herein.

Uses-Methods for Treatment

Without wishing to be bound by theory, it is believed that administration of 5-HT_{1A} and 5-HT_{1B} receptor antagonists prevents unwanted activity of the sacral reflex and/or cortical mechanisms that control micturition. Thus, it is contemplated that a wide range of neuromuscular dysfunctions of the lower urinary tract can be treated using the compounds of the present invention, including without limitation dysuria, incontinence and enuresis (overactive bladder). Dysuria includes urinary frequency, nocturia, urgency, reduced urinary compliance (reduced bladder storage capacity), difficulty in emptying the bladder, i.e., a suboptimal volume of urine is expelled during micturition. Incontinence syndromes include stress incontinence, urgency incontinence and enuresis incontinence, as well as mixed forms of incontinence. Enuresis refers to the involuntary passage of urine at night or during sleep.

### EXAMPLE 1

### Radioligand Binding to Recombinant 5-HT_{1A} Receptors

A. Method

Genomic clone G-21 coding for the human 5HT_{1A}-serotonergic receptor was stably transfected in a human cell line (HeLa). HeLa cells were grown as monolayers in Dulbecco's modified Eagle medium (DMEM), containing 10% fetal bovine serum, gentamicin (10 mg/ml) and 5% carbon dioxide, at 37° C. The cells were detached from the growth flask at 95% confluence by a cell scraper and were lysed in cold 5 mM Tris and 5 mM EDTA buffer (pH 7.4). The homogenates were centrifuged at 40000×g×20 minutes and the pellets were resuspended in a small volume of cold 5 mM Tris and 5 mM EDTA buffer (pH 7.4) and immediately frozen and stored at −70° C. until use. On the day of experiment, the cell membranes were resuspended in incubation buffer: 50 mM Tris HCl (pH 7.4), 2.5 mM MgCl₂, 10 mM pargyline (Fargin et al., Nature 335, 358, 1988). The membranes were incubated in a fmal volume of 1 ml for 30 minutes at 30° C. with 1 nM [³H]8—OH-DPAT, in the absence or presence of the test compounds. Non-specific binding was determined in the presence of 10 μM 5-HT. Incubation was stopped by addition of cold Tris-HCl buffer and rapid filtration through a 0.2%-polyethyleneimine-pretreated Whatman-GF/B or Schleicher-&-Schuell-GF52 filter.

B. Results

The affinity of the tested compounds was evaluated as inhibition of specific binding of the radioligand to 5-HT_{1A} receptors (IC₅₀) by using the non-linear curve-fitting program Allfit (De Lean et al., Am. J. Physiol. 235: E97, 1978). The IC₅₀ value was converted to an affinity constant (Ki) by the equation of Cheng & Prusoff (Biochem. Pharmacol. 22: 3099, 1973). The results are reported in Table 1.

TABLE 1
Binding affinity at 5HT_{1A} receptors. Data are expressed as Ki (nM)

| Compound | Affinity | Reference |
|---|---|---|
| N-[2-[4-(2-methoxy- | 0.33 | J. Pharmacol. Exp. Ther. |
| phenyl)piperazin-1- | | 290: 1258, 1999 |
| yl]ethyl]-N-(2- | | |
| pyridyl)cyclohexane | | |
| carboxamide | | |
| 1-[N-(2-nitrophenyl)-N- | 0.2 | J. Pharmacol. Exp. Ther. |
| cyclohexylcarbonyl-2- | | 299: 1027, 2001 |
| aminoethyl]-4-(2- | | |
| methoxyphenyl)piperazine | | |
| 1-cyclohexyl-4-[4-(2- | 0.68 | Recordati data on file |
| methoxyphenyl)piperazine- | | |
| 1-yl]-2-(2-pyridyl)butan-1- | | |
| one] | | |
| 1-cyclohexyl-4-[4-(2- | 0.15 | Recordati data on file |
| methoxy-phenyl)- | | |
| piperazin-1-yl]-2-pyridin- | | |
| 2-yl-butan-1-one | | |
| 1-[3-hydroxy-3,3 bis-(2- | 4.75 | Recordati data on file |
| pyridyl)propyl]-4-(4- | | |
| indolyl)piperazine | | |

### EXAMPLE 2

### Radioligand Binding to Recombinant 5-HT_{1B} Receptors

A. Method

C6-glial cells stably tranfected with a pcDNA3/h5-HT_{1B} plasmid were prepared as 5 monoclonal cell lines cultured (Pauwels et al., Naunyn-Schmied. Arch. Pharmacol. 353: 144, 1996), and used for radioligand binding experiments. On the day of experiments, the cell membrane expressing h5-HT_{1B}-receptors were resuspended in incubation buffer containing 50 mM Tris-HCl pH 7.7, 4 mM CaCl₂, 10 HM pargyline and 0.1% ascorbic acid. Membrane (20-80 μg protein), were incubated in a final volume of 0.5 ml for 30 min at 25° C., with 0.5 nM of [³H]corboxamidotryptamine, in absence or presence of competing drugs. Non-specific binding was determined in the presence of 10 μM serotonin. The incubation was stopped by addition of 3 ml ice-cold 50 mM Tris-HCI buffer pH 7.7 and rapid filtration over Whatman GF/B glass fibre filters using a Brandel harvester, washed and the radioactivity was counted by liquid scintillation spectrometry.

B. Results

The affinity of the tested compounds was evaluated as inhibition of specific binding of the radioligand to 5-HT_{1B} receptors (IC₅₀) and converted to an affinity constant (Ki) as in the Example 1. The results are reported in Table 2.

TABLE 2
Binding affinity at 5HT_{1B} receptors. Data are expressed as Ki (nM)

| Compound | Affinity | Reference |
|---|---|---|
| 3-[3- | 12.1 | J. Med. Chem. 40: 3542, 1997 |
| (dimethylamino)propyl]-4- | | |
| hydroxy-N-[4-(4- | | |
| pyridinyl)phenyl]benzamide | | |
| N-[3-[3- | 1 | Naunyn. Schmied. Arch. |
| (dimethylamino)ethoxy]-4- | | Pharmacol. 356(3): 312, 1997 |
| methoxyphenyl-2″-methyl- | | |
| 4′-(5-methyl-1,2,4- | | |
| oxadiazol-3-yl)-[1,1′- | | |
| biphenyl]-4-carboxamide | | |
| 1′-methyl-5-[(2′-methyl-4′- | 10 | Br. J. Pharmacol. 114: 1107, 1995 |
| (5-methyl-1,2,4-oxadiazol- | | |
| 3-yl) biphenyl-4- | | |
| yl]carbonyl]-2,3,6,7- | | |
| tetrahydrospiro[furo[2,3- | | |
| f]indole-3,4′-piperidine. | | |

### EXAMPLE 3

### Effect on Rhythmic Bladder-Voiding Contractions Induced by Bladder Filling in Anaesthetised Rats

A. Method

Female Sprague-Dawley rats weighing 225-275 g (Crl: CDo Br, Charles River Italia) were used. The animals were housed with free access to food and water and maintained on a forced 12-hour alternating light-dark cycle at 22-24° C. for at least one week, except during the experiment. The activity on rhythmic bladder voiding contractions was evaluated according to the method of Dray J., Pharmacol. Methods, 13:157, 1985, with some modifications as in Guarneri (Guarneri, Pharmacol. Res. 27:173, 1993. Briefly, the rats were anaesthetised by subcutaneous injection of 1.25 g/kg (5 ml/kg) urethane, after which the urinary bladder was catheterised via the urethra using PE 50 polyethylene tubing filled with physiological saline. The catheter was tied in place with a ligature around the external urethral orifice and was connected to conventional pressure transducers (Statham P23 ID/P23 XL). The intravesical pressure was displayed continuously on a chart recorder (Battaglia Rangoni KV 135 with DCI/TI amplifier). The bladder was then filled via the recording catheter by incremental volumes of warm (37° C.) saline until reflex bladder-voiding contractions occurred (usually 0.8-1.5 ml). For intravenous injection of bioactive compounds, PE 50 polyethylene tubing filled with physiological saline was inserted into the jugular vein. Tested compounds were administered or co-administered in solution in a final volume of 0.5 ml/kg.

From the cystometrogram, the number of contractions recorded 15 minutes before (basal values) and after treatment, as well as the mean amplitude of these contractions (mean height of the peaks in mmHg), was evaluated.

Since most compounds produced an effect that was relatively rapid in onset and led to a complete cessation of bladder contractions, bioactivity was conveniently estimated by measuring the duration of bladder quiescence (i.e., the length of the time in minutes during which no contractions occurred: disappearance time=DT). The administered doses (alone or in combination) of the tested compounds were chosen on the basis of previously published results obtained with the same compounds in the utilized model (Testa et al., J. Pharmacol. Exp. Ther. 290: 1258, 1999; Leonardi et al., J. Pharmacol. Exp. Ther. 299: 1027, 2001; Testa et al., BJU Int. 87: 256, 2001) or of their affinity for the 5HT_{1A} or 5-HT_{1B} receptor.

B. Results

The rapid distension of the urinary bladder in urethane-anaesthetised rats produced a series of rhythmic bladder-voiding contractions whose characteristics have been described (Maggi et al., Brain Res. 380:83, 1986; Maggi et al., J. Pharmacol. Exp. Ther., 230: 500, 1984).

The intravenous administration of vehicle did not block the bladder contractions.

The selective 5-HT_{1A} antagonists: a) N-[2-[4-(2-methoxy-phenyl)piperazin-1-yl]ethyl]-N-(2-pyridyl)cyclohexane carboxamidehydrochloride(0.01 mg/kg), b) 1-[N-(2-nitrophenyl)-N-cyclohexylcarbonyl-2-aminoethyl]-4-(2-methoxyphenyl)piperazine mesylate (0.03 mg/kg), c) 2-[4-[4-(7-chloro-2,3-dihydro-1,4-benzodioxin-5-yl)piperazin-1-yl]butyl]-1,2-benzisothiazol-3(2H)-one 1,1-dioxide dihydrochloride (0.01 mg/kg) , and d) 1-[3-hydroxy-3,3 bis-(2-pyridyl)propyl]-4-(4-indolyl)piperazine ( 0.1 mg/kg)alone blocked the bladder contractions for 10.39±1.45, 2.26±0.51, 6.96±1.05 and 9.29±0.80 min, respectively.

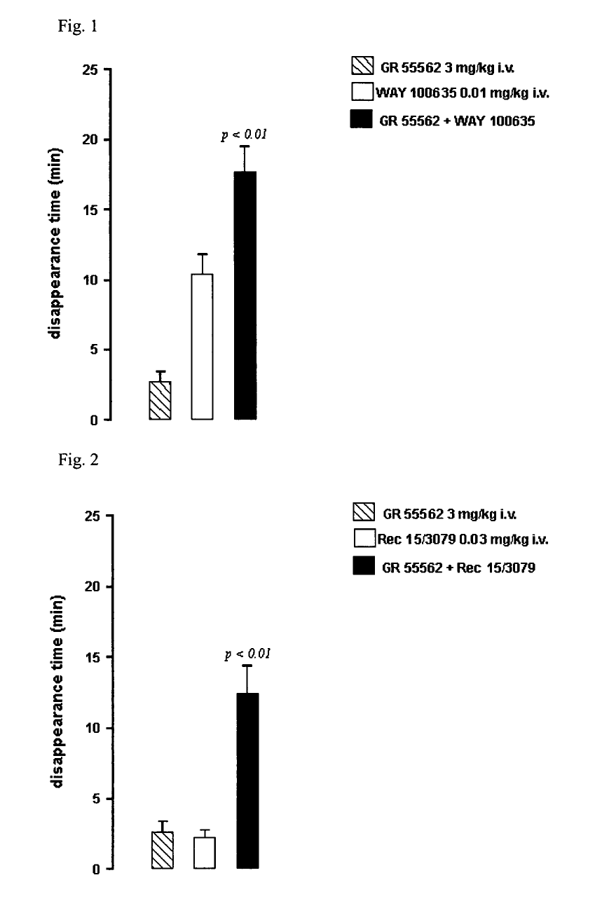

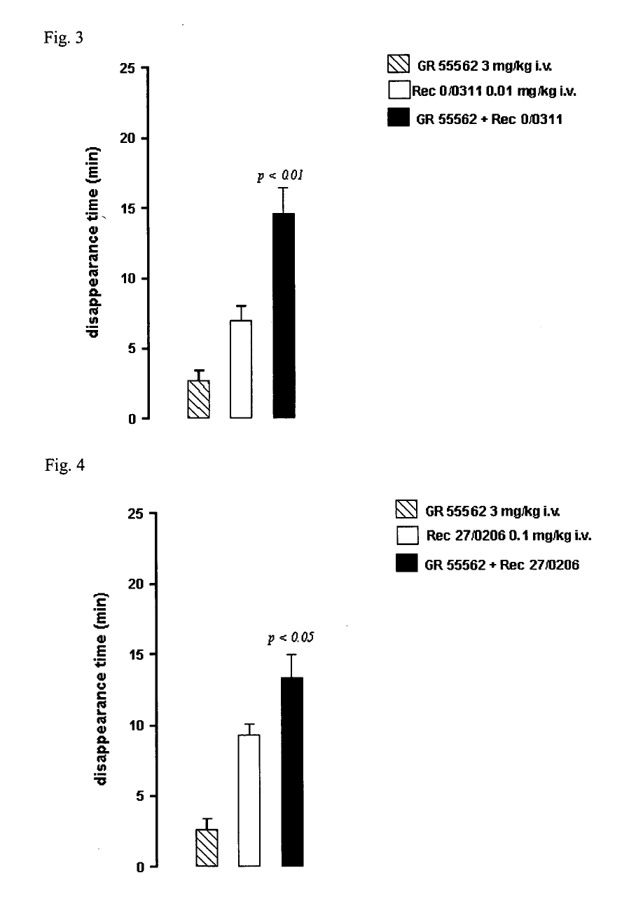

The co-administration of the above selective 5-HT_{1A} antagonists and 3-[3-(dimethylamino)propyl]-4-hydroxy-N-[4-(4-pyridinyl)phenyl]benzamide induced a block of bladder contractions (D.T.=17.7±1.83, 12.45±1.93, 14.6±1.85, and 13.31±1.67 min, respectively) significantly higher than that observed after administration of the selective antagonists alone (see FIG. 1-4).

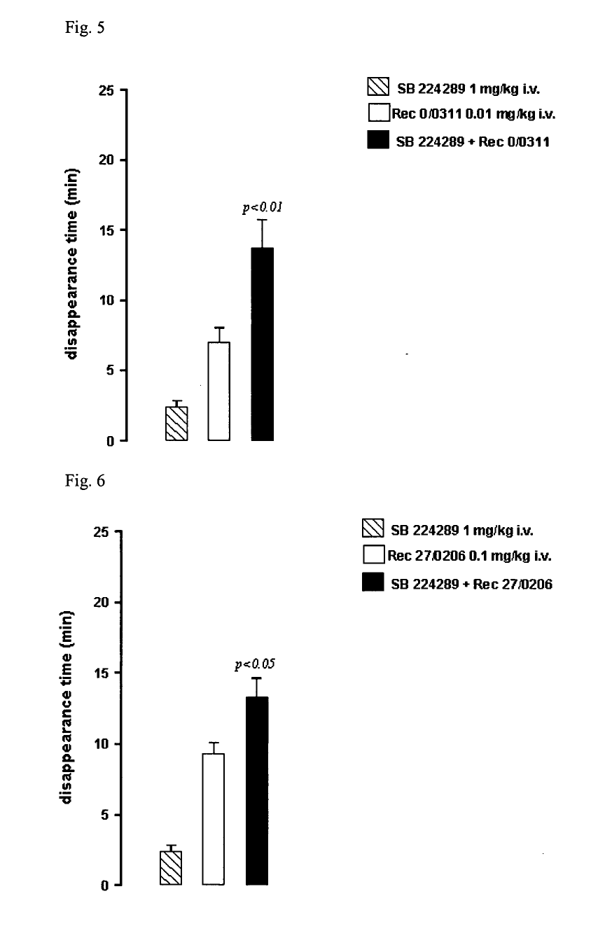

Similar results were obtained when the selective 5-HT_{1B} antagonist 1′-methyl-5-[(2′-methyl-4′-(5-methyl-1,2,4-oxadiazo]-3-yl) biphenyl-4-yl]carbonyl]-2,3,6,7-tetrahydrospiro[furo[2,3-f]indole-3,4′-piperidine hydrochloride (1.0 mg/kg) was administered alone (DT=2.40±0.46 min) or in combination with 2-[4-[4-(7-chloro-2,3-dihydro-1,4-benzodioxin-5-yl)piperazin-1-yl]butyl]-1,2-benzisothiazol-3(2H)-one 1,1-dioxide dihydrochloride (0.01 mg/kg), giving a DT of 13.69±2.05 min (p<0.01, FIG. 5), and in combination with 1-[3-hydroxy-3,3 bis-(2-pyridyl)propyl]-4-(4-indolyl)piperazine (0.1 mg/kg), giving a DT of 13.23±1.41 min (p<0.05, FIG. 6).

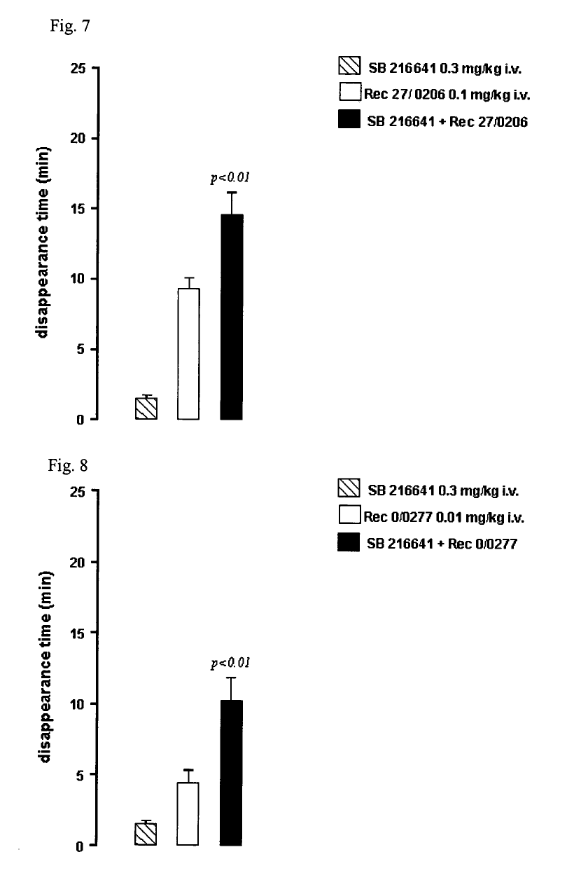

Furthermore, the selective 5-HT_{1B} antagonist N-[3-[2-(dimethylamino)ethoxy]-4-methoxyphenyl-2′-methyl-4′-(5-methyl-1,2,4-oxadiazol-3-yl)-[1,1′-biphenyl]-4-5 carboxamide hydrochloride (0.3 mg/kg) when administered alone gave a DT of 1.50±0.28 min, and when co-administered with 1-[3-hydroxy-3,3 bis-(2-pyridyl)propyl]-4-(4-indolyl)piperazine (0.1 mg/kg) and 1-cyclohexyl-4-[4-(2-methoxy-phenyl)-piperazin-1-yl]-2-pyridin-2-yl-butan-1-one gave (0.01 mg/kg) DT values of 14.58±1.62 (FIG. 7) and 10.2±1.68 (FIG. 8), respectively, both significantly (p<0.01) higher than the DT values observed after the administration of the 5-HT_{1A} antagonists alone (see FIGS. 7 and 8).

### EXAMPLE 4

### Effect on Cystometric Parameters in Conscious Rats After Intravenous Administration

A. Method

Male Sprague-Dawley rats [Crl: CD° (SD) BR] of 300-400 g supplied by Charles River Italia were used. The animals were housed with free access to food and water and maintained on a forced 12-hour-light/12-hour-dark cycle at 22-24° C. of temperature, except during the experiment. To quantify urodynamic parameters in conscious rats, cystometrographic studies were performed according to the procedure previously reported (Guarneri et al., Pharmacol. Res. 24: 175, 1991).

Briefly, the rats were anaesthesized by intraperitoneal administration of 3 ml/kg of Equithensin solution (pentobarbital 30 mg/kg and chloral hydrate 125 mg/kg) and placed in a supine position. An approximately-10-mm-long midline incision was made in the shaved and cleaned abdominal wall. The urinary bladder was gently freed from adhering tissues, emptied and then cannulated via an incision in the bladder body, using a polyethylene cannula (0.58-mm internal diameter, 0.96-mm external diameter) which was permanently sutured with silk thread. The cannula was exteriorised through a subcutaneous tunnel in the retroscapular area, where it was connected to a plastic adapter in order to avoid the risk of removal by the animal. For drug testing, the rats were utilised one day after implantation.

On the day of the experiment, the rats were placed in modified Bolhman cages, i.e., restraining cages that were large enough to permit the rats to adopt a normal crouched posture, but narrow enough to prevent turning around. After a stabilisation period of about 20 minutes, the free tip of the bladder cannula was connected through a T-shaped tube to a pressure transducer (Statham P23XL) and to a peristaltic pump (Gilson minipuls 2) for continuous infusion of a warm (37° C.) saline solution into the urinary bladder, at a constant rate of 0.1 ml/minute. The intraluminal-pressure signal during infusion of saline into the bladder was continuously recorded on a polygraph (Rectigraph-8K San-ei with BM614/2 amplifier from Biomedica Mangoni) and, from the cystometrogram, two urodynamic parameters were evaluated: bladder volume capacity (BVC) and micturition pressure (MP). BVC (in ml) is defmed as the volume of saline infused into the bladder necessary to induce detrusor contraction followed by micturition. MP (in mmHg) is defined as the maximal intravesical pressure caused by contraction during micturition. Basal BVC and MP values were evaluated as mean of the values observed in the cystometrograms recorded in an initial period of 30-60 minutes. At this point in the assay the test compounds were administered intravenously under continuous infusion of the bladder, and changes in BVC and MP were evaluated from the cystometrograms observed during 1, 2, 3, 4 and 5 hours after treatment. The compounds were administered in a volume of 2 ml/kg and groups of control animals received the same amount of vehicle.

Statistical Analysis

Data were expressed as mean±standard error. To compare the effects of the different treatments, for each rat the theoretical and practical AUC were evaluated. The theoretical AUC was the area under the curve having as abscissa the observation times and as ordinate the basal value of the considered parameter. The practical AUC was the area under the curve having as abscissa the observation times and as ordinate the observed value (at each time) of the considered parameter. For each animal, the Δ value “practical AUC-theoretical AUC” was evaluated.The difference between vehicle and active-treatments effect was evaluated by ONE WAY ANOVA followed by Tukey's test for multiple comparisons.

B. Results

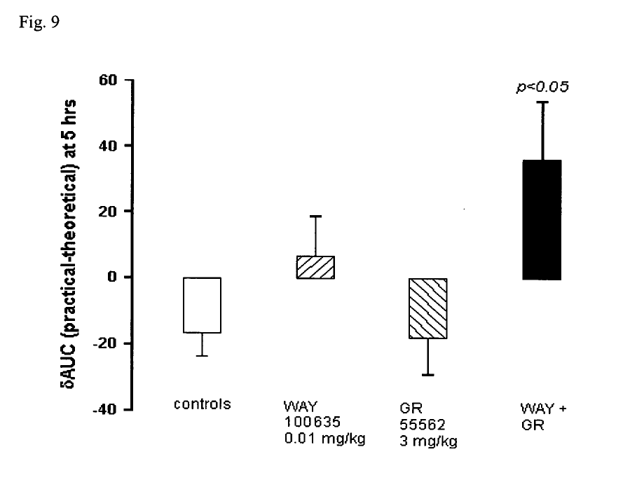

The effects of the administered doses of the tested compounds on the Δ values of BVC are shown in FIG. 9. The combination of the two antagonists induced an increase of BVC that was significantly (p<0.05) different from that induced by all the other treatments. Administration of the single antagonists alone induced changes of BVC that were not significantly different from those observed in the control group (animals treated with the vehicle). The changes induced by all the treatment on MP were not significant and practically the same observed in the control group.

1. A method for treating neuromuscular dysfunction of the lower urinary tract in a mammal in need of such treatment comprising administering an effective amount of:
(i) at least one compound that has 5HT_{1A} antagonist activity in combination with at least one compound that has 5HT_{1B} antagonist activity or
(ii) at least one compound that has both 5HT_{1A} and 5HT_{1B} antagonist activity,
or a pharmaceutically acceptable salt, enantiomer, diastereomer, N-oxide, crystalline form, hydrate, solvate, active metabolite or prodrug thereof of any of said compounds.

2. The method according to claim 1 wherein said at least one compound that has 5HT_{1A} antagonist activity is selected from the group consisting of compounds of Formula A, B, C, D, E, F, G, H, I, J and K:
wherein Formula A is a compound depicted by the formula 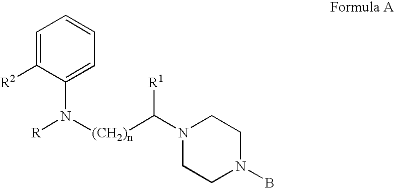
wherein for Formula A
R is a hydrogen atom, or alkylcarbonyl, cycloalkylcarbonyl, cycloalkylcarbonyl group substituted with one or more lower alkyl group or acyl group, or a monocyclic heteroarylcarbonyl group,
R¹ is a hydrogen atom or a lower alkyl group,
R² is an alkoxy, phenoxy, nitro, cyano, acyl, amino, acylarnino, alkylsulphonylamino, alkoxycarbonyl, aminocarbonyl, N-alkylaminocarbonyl, N,N-dialkylaminocarbonyl, N-acylaminocarbonyl, halogen, trifluoromethyl or polyfluoroalkoxy group,
B is a mono- or bi-cyclic aryl, each optionally substituted with one or more lower alkyl, lower alkoxy, polyhaloalkoxy, halogen, hydroxyl, nitro, cyano, amido, amino, alkylamino, acylamino, alkylsulphonylamino, lower acyloxy, lower N-alkylaminocarbonyloxy, N,N-dialkylaminocarbonyloxy or acyl group, a mono- or bicyclic heteroaryl, each optionally substituted with one or more alkyl, alkoxy, halogen, nitro, cyano, amido, amino, alkylamino, acylamino, alkylsulphonylamino or acyl group, or benzyl, optionally substituted with one or more alkyl, alkoxy, halogen, nitro, cyano, amido, amino, alkylamino, acylamino, alkylsulphonylamino, or acyl group,
and n is 1 or 2,
or a pharmaceutically acceptable salt, enantiomer, diastereomer, N-oxide, crystalline form, hydrate, solvate, active metabolite or prodrug thereof;
and wherein Formula B is a compound depicted by the formula 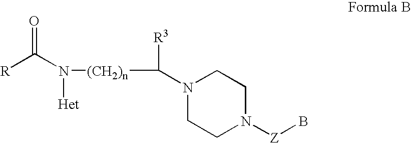
wherein for Formula B
n is 1 or 2,
Het is a monocyclic heteroaryl group,
R is a cycloalkyl or a monocyclic heteroaryl group,
R³ is a hydrogen atom or a lower alkyl group,
Z is a bond, —CH₂—, —CH₂CH₂—, —CH₂C(O)—, —CH₂CH(OH)—, —O—, —OCH₂—
or —C(O)— group, each of which is depicted with its left end being the end which attaches to the piperazine ring and the right end being the end which attaches to group B, B is selected from the group consisting of a heteroaryl, unsubstituted aryl, and substituted aryl groups, where substituted aryl is represented by the formula 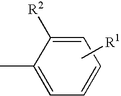
where R¹ is a single substituent selected from the group consisting of hydrogen, alkoxy, halogen, nitro, amino, acylamino, alkylamino, dialkylamino and alkylsulfonylamino, and R² is selected from the group consisting of alkoxy, polyfluoroalkoxy, cyano, halogen and aminocarbonyl,
and where the heteroaryl radical is selected from the group consisting of a mono or a bicyclic aromatic ring comprising from 5 to 12 ring atoms, where one or more of the ring atoms are selected from the group consisting of nitrogen, oxygen, and sulfur,
or a pharmaceutically acceptable salt, enantiomer, diastereomer, N-oxide, crystalline form, hydrate, solvate, active metabolite or prodrug thereof;
and wherein Formula C is a compound depicted by the formula 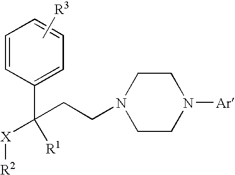
wherein for Formula C
Ar′ is a mono- or bi-cyclic aryl or heteroaryl radical, each of which may be optionally substituted independently with one to three substituents selected from the group consisting of hydrogen, C₁₋₆-alkyl, C₁₋₆-alkoxy, C₁₋₆-alkylthio, C₂₋₆-alkenyl, C₂₋₆-alkynyl, C₁₋₆-alkylhalo, C₃₋₈-cycloalkyl, C₃₋₈-cycloalkenyl or halo;
R¹ is hydrogen, C₁₋₆-alkyl, C₁₋₆-alkoxy, or C₁₋₆-alkylthio;
R² is phenyl, naphthyl or C₃₋₁₂-cycloalkyl, each of which may be optionally substituted independently with one or two substituents selected from the group consisting of hydrogen, C₁₋₆-alkyl, C₁₋₆-alkoxy, C₁₋₆-alkylthio, C₂₋₆-alkenyl, C₂₋₆-alkynyl, C₁₋₆-alkylhalo, C₃₋₈-cycloalkyl, C₃₋₈-cycloalkenyl and halo;
R³ is selected from the group consisting of hydrogen, C₁₋₆-alkyl, C₁₋₆-alkoxy, C₁₋₆-alkylthio, C₂₋₆-alkenyl, C₂₋₆-alkynyl, C₁₋₆-alkylhalo, C₃₋₈-cycloalkyl, C₃₋₈-cycloalkenyl and halo;
X is —C(═O)—, —CHOH— or —CH₂—;
or a pharmaceutically acceptable salt, enantiomer, diastereomer, N-oxide, crystalline form, hydrate, solvate, active metabolite or prodrug thereof;
and wherein Formula D is a compound depicted by the formula 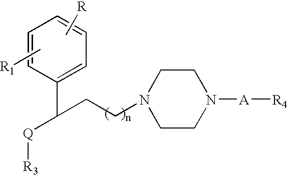
wherein for Formula D
R represents hydrogen or one or more substituents selected from the group consisting of (C₁-C₆)-alkyl, (C₁-C₆)-alkoxy, (C₁-C₆)-alkylthio, hydroxy, halo, (C₂-C₆)-alkenyl, (C₂-C₆)-alkynyl, (C₁-C₆)-haloalkyl, (C₁-C₆)-haloalkoxy, (C₁-C₆)-hydroxyalkyl, alkoxyalkyl, nitro, amino, (C₁-C₆)-aminoalkyl, (Ci-C₆)-alkylamino—(C₁-C₆)-alkyl, (C₁-C₆)-alkylamino, di-(C₁-C₆)-alkylamino, acylamino, (C₁-C₆)-alkylsulphonylamino, aminosulphonyl, (C₁-C₆)-alkylaminosulphonyl, cyano, aminocarbonyl, N—(C₁-C₆)-alkylaminocarbonyl, N,N-di-(C₁-C₆)-alkylaminocarbonyl, (C₁-C₆)-alkoxycarbonyl, (C₁-C₆)-alkylcarbonyl, alkylcarbonylalkyl, formyl, alkanoyloxyalkyl, (C₁-C₆)-alkylaminocarbonylamino, (C₁-C₆)-alkylsulphinyl, (C₁-C₆)-alkylsulphonyl, and N,N-di-(C₁-C₆)-alkylaminosulphonyl groups;
R₁ represents a member selected from the group consisting of hydrogen, cycloalkyl, aryl, aryloxy, aralkyl, aralkoxy, heterocyclic, heterocycloxy, heterocycloalkyl and heterocycloalkoxy groups, each group being optionally substituted with one or more substituent R, defmed as above;
Q represents —C(O)— or —CH(OR₂)— where R₂ represents a member selected from the group consisting of hydrogen, (C₁-C₆)-alkyl, (C₂-C₆)-alkenyl, (C₂-C₆)-alkynyl and cycloalkyl groups, wherein each group is optionally substituted with one or more groups selected from R₅ and R₆, where R₅ is selected from the group consisting of halo, (C₁-C₆)-alkoxy, (C₁-C₆)-haloalkoxy, cyano, (C₁-C₆)-alkoxycarbonyl, (C₁-C₆)-alkylcarbonyl, alkoxyalkyl, aminocarbonyl, N—(C₁-C₆)-alkylaminocarbonyl, N,N-di-(C₁-C₆)-alkylaminocarbonyl groups and R⁶ is selected from the group consisting of aryl, heteroaryl, aryloxy, heteroaryloxy, arylalkoxy, and heteroarylalkoxy groups, each optionally substituted with R, or R₂ represents —C(O)— (C₁-C₆)-alkyl, —C(O)O—(C₁-C₆)-alkyl, —C(O)NR₇R₈ or —C(S)NR₇R₈ wherein R₇ and R₈ are independently hydrogen or (C₁-C₆)-alkyl;
R₃ represents hydrogen or a (C₁-C₆)-alkyl, (C₂-C₆)-alkenyl, (C₂-C₆)-alkynyl, cycloalkyl, aryl or heterocycle group, each group being optionally substituted with one or more substituent R or R₁, defined as above;
R₄ represents an aryl or heterocyclic group, each being optionally substituted with one or more substituent R, defined as above;
A represents a bond or (CH₂),; and
n=1 or 2,
or an enantiomer, optical isomer, diastereomer, N-oxide, crystalline form, hydrate, solvate or pharmaceutically acceptable salt thereof;
and wherein Formula E is a compound depicted by the formula 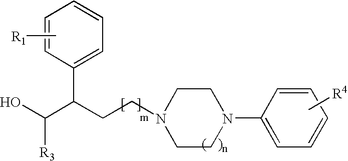
wherein for Formula E
R¹ represents a halogen atom,
R³ represents a (C₃-C₈)-cycloalkyl group,
R⁴ represents a (C₁-C₄)-alkoxy or (C₁-C₄)-haloalkoxy group,
m is 1 or 2, and
n is 1 or 2,
or a pharmaceutically acceptable salt, enantiomer, diastereomer, N-oxide, crystalline form, hydrate, solvate, active metabolite or prodrug thereof;
and wherein Formula F is a compound depicted by the formula 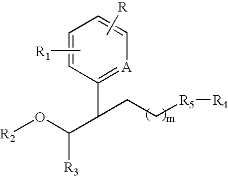
wherein for formula F
R is hydrogen or one or more substituents selected from the group consisting of alkyl, alkoxy, alkylthio, hydroxy, halo, alkenyl, alkynyl, polyhaloalkyl, monohaloalkoxy, polyhaloalkoxy, hydroxyalkyl, alkoxyalkyl, nitro, amino, aminoalkyl, alkylaminoalkyl, alkylamino, dialkylamino, acylamino, alkylsulphonylamino, aminosulphonyl, alkylaminosulphonyl, cyano, aminocarbonyl, N-alkylaminocarbonyl, N,N-dialkylaminocarbonyl, alkoxycarbonyl, alkylcarbonyl, alkylcarbonylalkyl, formyl, alkanoyloxyalkyl, alkylaminocarbonylamino, alkylsulphinyl, alkylsulphonyl, and N,N-dialkylaminosulphonyl groups;
R¹ is selected from the group consisting of hydrogen, cycloalkyl, aryl, aryloxy, aralkyl, aralkoxy, heterocyclic, heterocycloxy, heterocycloalkyl and heterocycloalkoxy groups, each group being optionally substituted with one or more substituent R, defined as above;
R² is selected from the group consisting of hydrogen, alkyl, alkenyl, alkynyl and cycloalkyl groups, wherein each group is optionally substituted with one or more groups selected from R⁸ and R⁹, where R⁸ is selected from the group consisting of halo, alkoxy, monohaloalkoxy, polyhaloalkoxy, cyano, alkoxycarbonyl, alkylcarbonyl, alkoxyalkyl, aminocarbonyl, N-alkylaminocarbonyl, N,N-dialkylaminocarbonyl groups and R₉ is selected from the group consisting of aryl, heteroaryl, aryloxy, heteroaryloxy, arylkoxy, and heteroarylkoxy groups, each optionally substituted with R¹;
R³ is alkyl, alkenyl, alkynyl, cycloalkyl, aryl or heterocycle, each being optionally substituted with one or more substituent R or R¹, defined as above;
R⁴ is aryl or heterocyclic, each being optionally substituted with one or more substituents R, defined as above;
A is CH or N,
R⁵ is 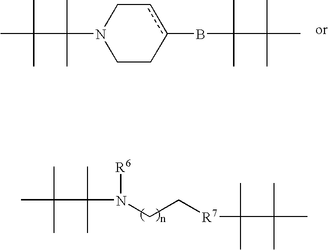
where R⁴ is bound to the right of each R⁵ group as depicted above
m and n are independently 1 or 2,
R⁶ is H or alkyl,
R⁷ is O, S, NR⁶ or CH₂; and
B is a bond, O, S, NR⁶or CH₂; and 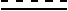
is a single or double bond,
or a pharmaceutically acceptable salt, enantiomer, diastereomer, N-oxide, crystalline form, hydrate, solvate, active metabolite or prodrug thereof;
and wherein Formula G is a compound depicted by the formula 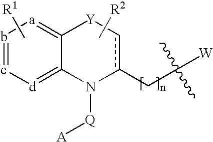
wherein for Formula G
W represents 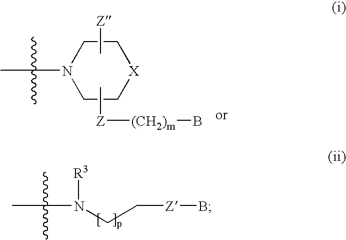
R¹ is one or more substituents selected from a group consisting of hydrogen, halogen, hydroxyl, alkyl, substituted alkyl, alkoxyl, substituted alkoxyl, nitro, aryl, substituted aryl, heterocycle, substituted heterocycle, alkenyl, substituted alkenyl, amino, alkylamino, dialkylamino, cyano, —SR³, —C(O)R³, —C(O)NR³R³, —NR³C(O)R³, —NR³SO₂R³, —NR³C(O)OR³ and —N(H)C(O)N(H)R³;
R³ is independently selected from a group consisting of hydrogen, alkyl, substituted alkyl, cycloalkyl, substituted cycloalkyl, aryl, substituted aryl, heterocycle and substituted heterocycle;
R² is one or two substituents selected from a group consisting of hydrogen, halogen, oxo, alkyl, substituted alkyl, alkenyl and substituted alkenyl groups;
Y represents a CH, CH₂, CR₂, CHR₂ group or a bond;
Q represents a carbonyl, thiocarbonyl or sulfonyl group;
A represents an alkyl, substituted alkyl, cycloalkyl, substituted cycloalkyl, alkenyl, substituted alkenyl, cycloalkenyl, substituted cycloalkenyl, aryl, substituted aryl, heterocycle, substituted heterocycle, alkylamino, substituted alkylamino, dialkylamino, substituted dialkylamino, cyclic amino, substituted cyclic amino, arylamino, substituted arylamino, arylalkylamino or substituted arylalkylamino group;
n is independently 1 or 2;
m is independently 0, 1 or 2;
p is independently 1, 2 or 3;
a, b, c and d are independently a carbon or nitrogen atom, or CH, CH₂ or NH group, with the proviso that no more than two of a, b, c and d may simultaneously be a nitrogen atom and/or NH,
X represents a bond, CH, CH₂, SO or SO₂ group or a carbon, nitrogen or sulphur atom and, when X is a nitrogen atom or CH group, the -Z-(CH₂)ₘ—B group is bound to said nitrogen atom or CH group, and when X is a carbon atom Z″ is not a hydrogen atom or oxo group and the Z-(CH₂)ₘ—B and Z″ groups are bound to said carbon;
Z represents a bond, an oxygen or sulphur atom or —CH(OH)—, —C(O)—NR³C(O)—, —NR³—C(O)—NR³—, or —NR³— group;
Z′ represents a bond or an oxygen or sulphur atom;
Z″ represents a hydrogen atom or hydroxyl, oxo, alkylcarbonyl or cyano group,
B represents a monocyclic aryl, substituted monocyclic aryl, bicyclic aryl, substituted bicyclic aryl, monocyclic heterocycle, substituted monocyclic heterocycle, bicyclic heterocycle or substituted bicyclic heterocycle; 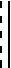
represents a single or double bond and, when Y═CH, the double bond is shifted so as to contain it;
or a pharmaceutically acceptable salt, enantiomer, diastereomer, N-oxide, crystalline form, hydrate, solvate, active metabolite or prodrug thereof;
and wherein Formula H is a compound depicted by the formula 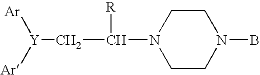
wherein for Formula H
each of Ar and Ar′ is independently selected from a group consisting of phenyl and pyridyl, each optionally substituted by one or more members selected from the group consisting of alkyl, alkoxy, cyano, nitro, amino, alkylsulfonylamino, and alkylamino;
Y is a member selected from the group consisting of nitrogen atom, CH, C—OH, C—CN and C—CONH₂;
R is a hydrogen atom or a lower alkyl group; and
B is (a) phenyl substituted with one or more substituents selected from the group consisting of alkoxy, halogen, cyano, nitro, amino, alkylsulfonylamino and alkylamino; (b) naphthyl, optionally substituted with one or more substituents selected from the group consisting of alkyl, alkoxy, halogen, cyano, nitro, amino, alkylsulfonylamino and alkylamino groups; (c) benzodioxanyl; or (d) indolyl;
or a pharmaceutically acceptable salt, enantiomer, diastereomer, N-oxide, crystalline form, hydrate, solvate, active metabolite or prodrug thereof;
and wherein Formula I is a compound depicted by the formula 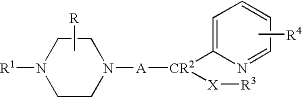
wherein for Formula I
R represents hydrogen or one or two same or different C₁₋₆-alkyl groups;
R¹ is a mono- or bicyclic aryl or heteroaryl radical;
R₂ is hydrogen or lower alkyl;
R³ is lower alkyl or cycloalkyl;
R⁴ is hydrogen or lower alkyl;
A is an alkylene chain of 1 to 3 carbon atoms optionally substituted by one or more lower alkyl groups; and
X is —CO—, —CR⁵OH— (where R⁵ is hydrogen, lower alkyl or cycloalkyl), —S—, —SO— or —SO₂— or X can also be —(CH₂)ₙ— (where n is 0, 1 or 2) when R³ is cycloalkyl;
or a pharmaceutically acceptable salt, enantiomer, diastereomer, N-oxide, crystalline form, hydrate, solvate, active metabolite or prodrug thereof;
and wherein Formula J is a compound depicted by the formula 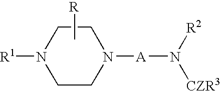
wherein for Formula J
A is an alkylene chain of 2 to 4 carbon atoms optionally substituted by one or more lower alkyl groups,
Z is oxygen or sulphur,
R is hydrogen or lower alkyl,
R¹ is a mono or bicyclic aryl or heteroaryl radical,
R² is a mono or bicyclic heteroaryl radical, and
R³ is hydrogen, lower alkyl, cycloalkyl, cycloalkenyl, cycloalkyl(lower)alkyl, aryl, aryl(lower)alkyl, heteroaryl, heteroaryl-(lower)alkyl, a group of formula —NR⁴R⁵, where R⁴ is hydrogen, lower alkyl, aryl or aryl-(lower)alkyl and R⁵ is hydrogen, lower alkyl, —CO(lower)alkyl, aryl, COaryl, aryl(lower)alkyl, cycloalkyl or cycloalkyl-(lower)alkyl or R⁴ and R⁵ together with the nitrogen atom to which they are attached represent a saturated heterocyclic ring which may contain a further hetero atom or R³ is a group of formula OR⁶, where R⁶ is lower alkyl, cycloalkyl, cycloalkyl(lower)alkyl, aryl, aryl(lower)alkyl, heteroaryl or heteroaryl(lower)alkyl;
or a pharmaceutically acceptable salt, enantiomer, diastereomer, N-oxide, crystalline form, hydrate, solvate, active metabolite or prodrug thereof;
and wherein Formula K is a compound depicted by the formula 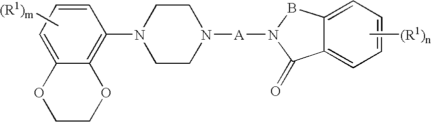
wherein for Formula K
R¹ is halogen, lower alkyl or alkoxy, hydroxy, trifluoromethyl or cyano,
m has the value 1 or 2,
n has the value 0 or 1,
A represents a C₂₋₆ alkylene chain which may be substituted with one more R substituent selected from the group consisting of lower alkyl and monocyclic (hetero)aryl groups, and
B is methylene, ethylene, carbonyl, sulfinyl, sulfonyl, or sulfur, and salts thereof.

3. The method according to claim 1 or claim 2 wherein said at least one compound that has 5HT_{1B} antagonist activity is selected from the group consisting of compounds of Formula L, M, N, O, P ,Q, R and S
wherein Formula L is a compound depicted by the formula 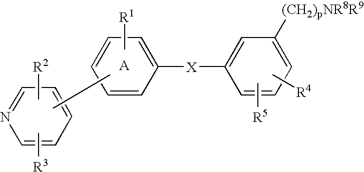
wherein for Formula L
R¹ represents a hydrogen or halogen atom, or C₁₋₆-alkyl, or C₁₋₆-alkoxy group;
R² and R³ independently represent a hydrogen or halogen atom, or a C₁₋₆-alkyl, hydroxyC₁₋₆-alkyl, C₁₋₆-alkoxyC₁₋₆-alkyl, C₁₋₆-alkoxy, hydroxy, —CN, —NO₂, —CO₂R⁶, —COR⁶, —C(O)NR⁶R⁷, OR —(CH₂)ₘOC(O)C₁₋₄alkyl group;
R⁴ and R⁵ independently represent a hydrogen or halogen atom, or a hydroxy, C₁₋₆-alkyl, or C₁₋₆-alkoxy group;
R⁶, R⁷, R⁸, and R⁹ independently represent a hydrogen atom or a C₁₋₆-alkyl group;
or —NR⁶R⁷ forms a saturated heterocyclic ring which has 5 or 6 members which, when there are 6 ring members, may optionally contain in the ring one oxygen or sulfur atom;
X represents —C(O)NH—, —NHC(O)—, —CH₂NH— or —NHCH₂—;
m represents zero or an integer from 1 to 3; and
p represents an integer from 2 to 4,
or a pharmaceutically acceptable salt, enantiomer, diastereomer, N-oxide, crystalline form, hydrate, solvate, active metabolite or prodrug thereof;
and wherein Formula M is a compound depicted by the formula 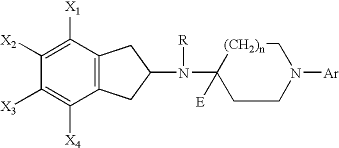
wherein for Formula M
n represents 1 or 2;
Ar represents 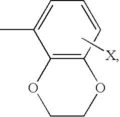
wherein X represents a hydrogen or fluorine atom, or Ar represents 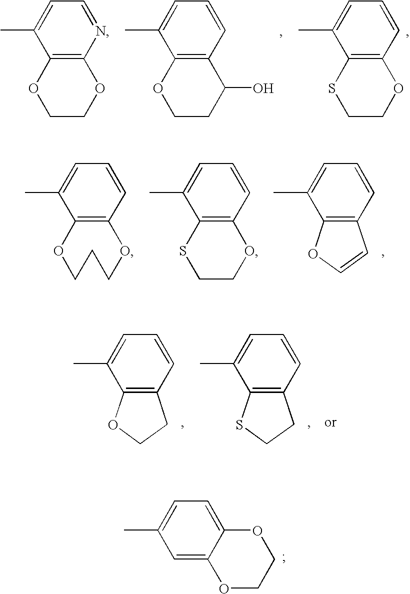
R represents a hydrogen atom, or C₁₋₅-alkyl, or aralkyl group,
E represents a hydrogen atom or methyl group, and
X₁, X₂, X₃, and X₄ independently represent a hydrogen or halogen atom, or C₁-C₅-alkyl, C₁-C₅-alkoxy, trifluoromethyl, hydroxy, cyano, nitro, —NR¹R², —C(O)NR¹R², —COOR³, —OC(O)R⁴, 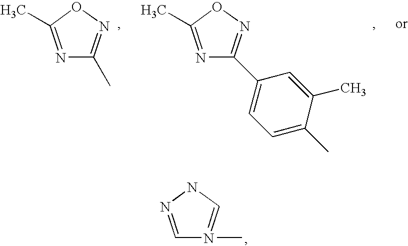
R¹, R², and R³ independently represent a hydrogen atom or C₁-C₅-alkyl group, and R⁴ represents a C₁-C₅-alkyl group,
or, independently, a pair of X₁ and X₂, X₂ and X₃, or X₃ and X₄, together with the carbon atoms of the phenyl ring to which they are attached, form a 5-membered or 6-membered ring composed of atoms selected from the atoms carbon, oxygen, nitrogen, and sulfur,
or a pharmaceutically acceptable salt, enantiomer, diastereomer, N-oxide, crystalline form, hydrate, solvate, active metabolite or prodrug thereof,
and wherein Formula N is a compound depicted by the formula 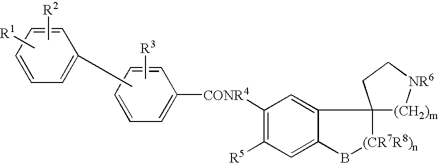
wherein for Formula N
R¹ represents a hydrogen or halogen atom, or C₁₋₆-alkyl, C₃₋₆-cycloalkyl, COC₁₋₆-alkyl, C₁₋₆-alkoxy, hydroxy, hydroxyC₁₋₆-alkyl, hydroxyC₁₋₆-alkoxy, C₁₋₆-alkoxyC₁₋₆alkoxy, acyl, nitro, trifluoromethyl, cyano, SR⁹, SOR⁹, SO₂R⁹, NR⁹CONR¹⁰R¹¹, NR¹⁰SO₂R¹¹, SO₂NR¹⁰R¹¹, CO₂R¹⁰, CONR¹⁰R¹¹, CO₂NRIOR¹¹, CONR¹⁰(CH₂)ₐCO₂R¹¹, (CH₂)ₐNR¹⁰R¹¹, (CH₂)ₐCONR¹⁰R¹¹, (CH₂)ₐNR¹⁰COR¹¹, (CH₂)aCO₂C₁₋₆-alkyl, CO₂(CH₂)ₐOR¹⁰, NR¹⁰R¹¹, N═CNR⁹NR¹⁰R¹¹, NR¹⁰CO(CH₂)ₐNR¹⁰R¹¹, NR¹⁰CO₂R¹¹, CONHNR¹⁰R¹¹, CR¹⁰═NOR¹¹, CNR¹⁰═NOR¹¹, where R⁹, R¹⁰, and R¹¹ are independently hydrogen or C₁₋₆-alkyl and “a” is an integer from 1 to 4; or R¹ is a 5- to 7-membered heterocyclic ring containing 1 to 4 heteroatoms selected from oxygen, nitrogen, and sulfur, optionally substituted with one or more substituents defined as R² or R³ below;
R² and R³ are independently hydrogen, halogen, C₁₋₆-alkyl, C₃₋₆-cycloaklyl, C₃₋₆-cycloalkenyl, C₁₋₆-alkoxy, hydroxyC₁₋₆-alkyl, C₁₋₆-alkylOC₁₋₆-alkyl, acyl, aryl, acyloxy, hydroxy, nitro, trifluoromethyl, cyano, CO₂R¹⁰, CONR¹⁰R¹¹, NR¹⁰R¹¹, where R¹⁰ and R¹¹ are independently hydrogen or C₁₋₆-alkyl;
R⁴ is hydrogen or C₁₋₆-alkyl;
R⁵ is hydrogen or C₁₋₆-alkyl, or R⁴ and R₅ together from a group -A-, where A is (CR¹³R¹⁴)_{q} where q is 2, 3, or 4, and R¹³ and R¹⁴ are independently hydrogen or C₁₋₆-alkyl or A is (CR¹³R¹⁴)ᵣ-D where r is 0, 1, 2, or 3 and D is oxygen, sulfur, or CR¹³═CR¹⁴;
R⁶ is a group —(CH₂)ₚ—R¹⁵, where R¹⁵ is OR 6 or SR¹⁶ where R¹⁶ is hydrogen or C₁₋₆-alkyl or R¹⁵ is NR¹⁰R¹¹ where R¹⁰ and R¹¹ are as defmed for R¹;
R⁷ and R⁸ are independently hydrogen or C₁₋₆-alkyl;
B is oxygen, CR¹⁷R¹⁸ or NR¹⁹ where R¹⁷, R¹⁸, and R¹⁹ are independently hydrogen or C₁₋₆-alkyl or B is a group S(O)_{b} where b is 1, 2, or 3;
m is 1,2, or 3; and
n is 1,2,or3,
or a pharmaceutically acceptable salt, enantiomer, diastereomer, N-oxide, crystalline form, hydrate, solvate, active metabolite or prodrug thereof;
and wherein Formula O is a compound depicted by the formula 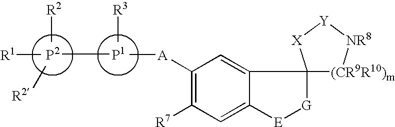
wherein for formula O
P¹ and P² are independently phenyl, bicyclic aryl, a 5- to 7-membered heterocyclic ring containing 1 to 4 heteroatoms selected from oxygen, nitrogen, or sulfur, or a bicyclic heterocyclic ring containing one to three heteroatoms selected from oxygen, nitrogen, or sulfur;
R¹ represents a hydrogen or halogen atom, or C₁₋₆-alkyl, C₃₋₆-cycloalkyl, COC₁₋₆-alkyl, C₁₋₆-alkoxy, hydroxy, hydroxyC₁₋₆-alkyl, hydroxyC₁₋₆-alkoxy, C₁₋₆-alkoxyC₁₋₆alkoxy, acyl, nitro, trifluoromethyl, cyano, SR⁹, SOR⁹, SO₂R⁹, SO₂NR¹⁰R¹¹, CO₂R¹⁰, NR¹⁰SO₂R¹¹, CONR¹⁰R₁₁, CO₂NR¹⁰R¹¹, CONR¹⁰(CH₂),CO₂R¹¹, (CH₂),NR¹⁰R¹¹, (CH₂)ₚCONR¹⁰R¹¹, (CH₂)ₚNR¹⁰COR¹¹, CONR¹⁰(CH₂)ₚCO₂C₁₋₆-alkyl, CO₂(CH₂)ₚOR¹⁰, CONHNR¹⁰R¹¹, NR¹⁰R¹¹, N═CNR⁹NR¹⁰R¹¹, NR¹⁰CO₂R¹¹, NR¹⁰CO(CH₂)ₚNR¹⁰R¹¹, NR¹⁰CONR¹⁰R¹¹, CR¹⁰═NOR¹¹, CNR¹⁰═NOR¹¹, or NR¹²COR¹³, where R⁹, R¹⁰, and R¹¹ are independently hydrogen or C₁₋₆-alkyl, p is 1 to 4, R¹² is hydrogen, C₁₋₆-alkyl or together with R² forms a group (CH₂)_{q} where q is 2, 3, or 4 and R¹³ is hydrogen, C₁₋₆-alkyl, aryl, or aryl substituted with one or more substituents selected from R² and R³, as defined below; or R¹ is a 5- to 7-membered heterocyclic ring containing 1 to 4 heteroatoms selected from oxygen, nitrogen, or sulfur, optionally substituted with one or more substituents selected from R² and R³, as defined below;
R² and R³ are independently hydrogen, halogen, C₁₋₆-alkyl, C₃₋₆-cycloalkyl, C₃₋₆-cycloalkenyl, C₁₋₆-alkoxy, hydroxyC₁₋₆-alkyl, C₁₋₆alkylOC₁₋₆-alkyl, acyl, aryl, acyloxy, hydroxy, nitro, trifluoromethyl, cyano, CO₂R¹¹, CONR¹⁰R¹¹, NR¹⁰R¹¹ where R¹⁰ and R¹¹ are independently hydrogen or C₁₋₆-alkyl, or R² and R³ together form a group —(CH₂)ᵣ—R¹⁴—(CH₂)ₛ— where R¹⁴ is O, S, CH₂, or NR¹⁵ where R¹⁵ is hydrogen or C₁₋₆-alkyl and r and s are independently 0, 1, or 2;
A is a group DR⁶—C(═B)— or a group —C(═B)-DR⁶ where B is oxygen or sulfur and D is nitrogen, carbon or a CH group; and
R⁶ is hydrogen or C₁₋₆-alkyl and R⁷ is C₁₋₆-alkyl, C₁₋₆-alkoxy, or halogen, or R⁶ and R⁷ together form a group -M- where M is (CR¹⁶R¹⁷)t where t is 1, 2, or 3 and R¹⁶ and R¹⁷ are independently hydrogen or C₁₋₆-alkyl or M is (CR1⁶R¹⁷)_-J wherein u is 0, 1, or 2 and J is oxygen, sulfur, CR¹⁶═CR¹⁷, CR¹⁶═N, or N═N;
R⁸ is hydrogen or C₁₋₆-alkyl;
R⁹ and R¹⁰ are independently hydrogen or C₁₋₆-alkyl;
E is oxygen, CR¹⁸R¹⁹, or NR²⁰ where R¹⁸, R¹⁹ and R²⁰ are independently hydrogen or C₁₋₆-alkyl or E is S(O)ᵥ where v is 0, 1, or 2;
G is C═O or CR²¹R²² where R²¹ and R²² are independently hydrogen or C₁₋₆-alkyl;
X and Y are independently CR⁹R¹⁰ where R⁹ and R¹⁰ are defmed as above; and m is 1, 2, or 3, provided that P¹ and P² are not both phenyl,
or a pharmaceutically acceptable salt, enantiomer, diastereomer, N-oxide, crystalline form, hydrate, solvate, active metabolite or prodrug thereof;
and wherein Formula P is a compound depicted by the formula 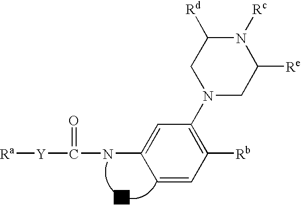
wherein for formula P
R^{a} is a group of formula (i) 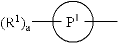
wherein
P¹ is phenyl, naphthyl, or heteroaryl;
R¹ is halogen, C₁₋₆-alkyl, C₃₋₆-cycloalkyl, COC₁₋₆-alkyl, C₁₋₆-alkoxy, hydroxy, hydroxyC₁₋₆-alkyl, nitro, trifluoromethyl, cyano, SR⁶, SOR⁶, SO₂R⁶, SO₂NR⁶R⁷, CO₂R⁶, CONR⁶R⁷, OCONR⁶R⁷, NR⁶R⁷, NR⁶CO₂R⁷, NR⁶CONR⁷R⁸, CR⁶═NOR⁷, where R⁶, R7 and R⁸ are independently hydrogen or CI.₆-alkyl;
a is 0, 1, 2 or 3;
or R^{a} is a group of formula (ii) 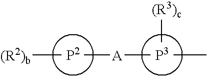
wherein
P² is phenyl, naphthyl, heteroaryl, or a 5- to 7-membered heterocyclic ring;
P³ is phenyl, naphthyl, or heteroaryl;
A is a bond or oxygen, carbonyl, CH₂ or NR⁴ where R⁴ is hydrogen or C₁₋₆-alkyl;
R² is as defined above for R¹ in formula (i) or R² is heteroaryl, optionally substituted by C₁₋₆-alkyl, halogen, or COC₁₋₆-alkyl, or is a 5- to 7-membered heterocyclic ring optionally subsituted by oxo;
R³ is halogen, C₁₋₆-alkyl, C₃-6-cycloalkyl, C₁₋₆-alkoxy, COC₁₋₆-alkyl, hydroxy, nitro, trifluoromethyl, cyano, CO₂R⁶, CONR⁶R⁷, NR⁶R⁷ where R⁶ and R⁷ are as defined above;
b and c are independently 0, 1, 2, or 3;
Y is a single bond, CH₂, 0, or NR⁵ where R⁵ is hydrogen or C₁₋₆-alkyl;
W is —(CR⁹R¹⁰)ₜ— where t is 2, 3, or 4 and R⁹ and R¹⁰ are independently hydrogen or C₁₋₆-alkyl or W is a group CH═CH;
R^{b} is hydrogen, halogen, hydroxy, C₁₋₆-alkyl, trifluoromethyl, COC₁₋₆-alkyl, cyano or C₁₋₆-alkoxy;
R^{c} is hydrogen or C₁₋₆-alkyl; and
R^{d} and R^{e} are independently C₁₋₄-alkyl,
or a pharmaceutically acceptable salt, enanfiomer, diastereomer, N-oxide, crystalline form, hydrate, solvate, active metabolite or prodrug thereof;
and wherein Formula Q is a compound depicted by the formula 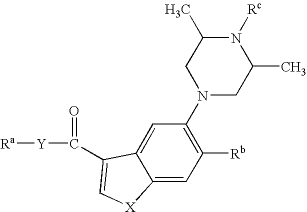
wherein for Formula Q
R^{a} is a group of formula (i) 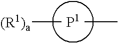
P¹ is phenyl, naphthyl, or heteroaryl;
R¹ is halogen, C₁₋₆-alkyl, C₃₋₆-cycloalkyl, COC₁₋₆-alkyl, C₁₋₆-alkoxy, hydroxy, hydroxyC₁₋₆-alkyl, nitro, haloC₁₋₆-alkyl, cyano, SR⁶, SOR⁶, SO₂R⁶, SO₂NR⁶R⁷, CO₂R, CONR⁶R⁷, OCONR⁶R⁷, NR⁶R⁷, NR⁶CO₂R⁷, NR6CONR⁷R⁸, CR⁶═NOR⁷, where R⁶ R⁷ and R⁸ are independently hydrogen or C₁₋₆-alkyl;
a is 0, 1, 2 or 3;
or R^{a} is a group of formula (ii) 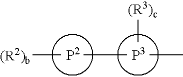
wherein
P2 is phenyl, naphthyl, heteroaryl, or a 5- to 7-membered heterocyclic ring;
P³ is phenyl, naphthyl, or heteroaryl;
R² is as defined above for R¹ in formula (i) or R² is heteroaryl, optionally substituted by C₁₋₆-alkyl, halogen, or COC₁₋₆-alkyl, or is a 5- to 7-membered heterocyclic ring optionally subsituted by oxo;
R³ is halogen, C₁₋₆-alkyl, C₃-6-cycloalkyl, C₁₋₆-alkoxy, COC₁₋₆-alkyl, hydroxy, nitro, haloC₁₋₆-alkyl, cyano, CO₂R⁶, CONR⁶R⁷, NR⁶R⁷ where R⁶ and R⁷ are as defined above;
b and c are independently 0, 1, 2, or 3;
Y is a single bond, CH₂, or NH;
X is oxygen, sulfur, or N—R⁵ where R⁵ is hydrogen or C₁₋₆-alkyl;
R^{b} is hydrogen, halogen, C₁₋₆-alkyl, haloC₁₋₆-alkyl, COC₁₋₆-alkyl, or cyano; and
R^{c} is hydrogen or C₁₋₆-alkyl,
or a pharmaceutically acceptable salt, enantiomer, diastereomer, N-oxide, crystalline form, hydrate, solvate, active metabolite or prodrug thereof;
and wherein Formula R is a compound depicted by the formula 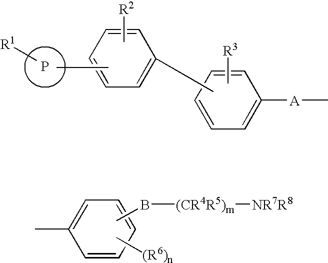
wherein for Formula R
P is a 5 to 7-membered heterocyclic ring containing 1 to 3 heteroatoms selected from oxygen, nitrogen or sulphur,
R¹, R² and R³ are independently hydrogen, halogen, C₁₋₆-alkyl, C₃-6-cycloalkyl, C₃₋₆-cycloalkenyl, C₁₋₆-alkoxy, hydroxy, C₁₋₆-alkyl, C₁₋₆-alkyl, OC₁₋₆-alkyl, acyl, aryl, acyloxy, hydroxy, nitro, trifluoromethyl, cyano, CO₂R⁹, CONR¹⁰R¹¹, NR¹⁰R¹¹ where R⁹, R¹⁰ and R are independently hydrogen or C₁₋₆-alkyl;
R⁴ and R⁵ are independently hydrogen or C₁₋₆ alkyl;
R⁶ is hydrogen, halogen, hydroxy, C₁₋₆ alkyl or C₁₋₆ alkoxy;
R⁷ and R⁸ are independently hydrogen, C₁₋₆ alkyl, aralkyl, or together with the nitrogen atom to which they are attached form an optionally substituted 5- to 7-membered heterocyclic ring containing one or two heteroatoms selected from oxygen, nitrogen or sulphur;
A is CONH or NHCO;
B is oxygen, S(O)ₚwhere p is 0, 1 or 2, NR¹² where R¹² is hydrogen, C₁₋₆-alkyl or phenylC₁₋₆-alkyl, or B is CR⁴═CR⁵ or CR⁴R⁵ where R⁴ and R⁵ are independently hydrogen or C₁₋₆-alkyl;
m is an integer from 1 to 4;
and n is an integer from 1 or 2;
or a pharmaceutically acceptable salt, enantiomer, diastereomer, N-oxide, crystalline form, hydrate, solvate, active metabolite or prodrug thereof;
and wherein Formula S is a compound depicted by the formula 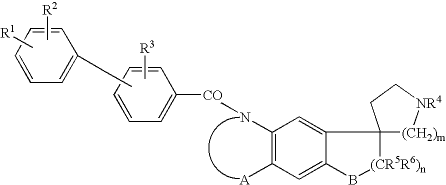
wherein for Formula S
R¹ is hydrogen, halogen, C₁₋₆-alkyl, C₃₋₆-cycloalkyl, COCI-6-alkyl, C₁₋₆-alkoxy, hydroxy, hydroxyC₁₋₆alkyl, hydroxyC₁₋₆alkoxy, C₁₋₆alkoxyC₁₋₆alkoxy, acyl, nitro, trifluoromethyl, cyano, SR⁹, SOR⁹, SO₂R⁹, SO₂NR¹⁰R¹¹, CO₂R¹⁰, NR¹⁰SO₂R¹¹, CONR¹⁰R¹¹, CO₂NR¹⁰R¹¹, CONR¹⁰(CH₂)ₚCO₂R¹¹, (CH₂)ₚNR¹⁰R¹¹, (CH₂)ₚCONR¹⁰R¹¹, (CH₂)ₚNR¹⁰COR¹¹, (CH₂)ₚCO₂C₁₋₆alkyl, CO₂(CH₂)ₚOR¹⁰, CONHNR¹⁰R¹¹, NR¹⁰R¹¹, NR¹⁰CO₂R¹¹, NR¹⁰CO₂R¹¹, NR¹⁰CO(CH₂)ₚNR¹⁰R¹¹, NR¹⁰CONR¹⁰R¹¹, CR¹⁰═NOR¹¹, CNR¹⁰═NOR¹¹, where R⁹, R¹⁰ and R¹¹ are independently hydrogen or C₁₋₆alkyl and p is 1 to 4; or R¹ is an optionally substituted 5 to 7-membered heterocyclic ring containing 1 to 4 heteroatoms selected from oxygen, nitrogen or sulphur;
R² and R³ are independently hydrogen, halogen, C₁₋₆-alkyl, C₃₋₆-cycloalkyl, C₃₋₆-cycloalkenyl, C₁₋₆-alkoxy, hydroxyC₁₋₆-alkyl, C₁₋₆-alkyl, OC₁₋₆-alkyl, acyl, aryl, acyloxy, hydroxy, nitro, trifluoromethyl, cyano, CO₂R¹⁰, CONR¹⁰R¹¹, NR¹⁰R¹¹ where R¹⁰ and R¹¹ are independently hydrogen or C₁₋₆-alkyl;
R⁴ is hydrogen or C₁₋₆-alkyl;
R⁵ and R⁶ are independently hydrogen or C₁₋₆-alkyl;
A is (CR¹³R¹⁴) q where q is 2, 3 or 4 and R¹³ and R¹⁴ are independently hydrogen or C₁₋₆-alkyl or A is (CR¹³R¹⁴)ᵣ-D where r is 0, 1, 2 or 3 and D is oxygen, sulphur or CR¹³═CR¹⁴.
B is oxygen, CR¹⁵R¹⁶ or NR¹⁷ where R¹⁵, R¹⁶ and R¹⁷ are independently hydrogen or C₁₋₆alkyl or B is S(O) b where b is 0, 1 or 2;
m is 1, 2 or 3;
n is 1, 2 or 3;
or a salt or N-oxide thereof.

4. The method according to claim 3 wherein said compound that has both 5HT_{1A} and 5HT_{1B} antagonist activity is selected from the group consisting of compounds of Formnula T, U, V or W:
wherein Formula T is a compound depicted by the formula 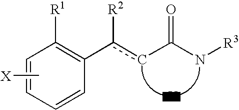
wherein for Formula T
R¹ is a member selected from the group consisting of G¹, G², G³, G⁴, G⁵, G⁶ and G⁷, 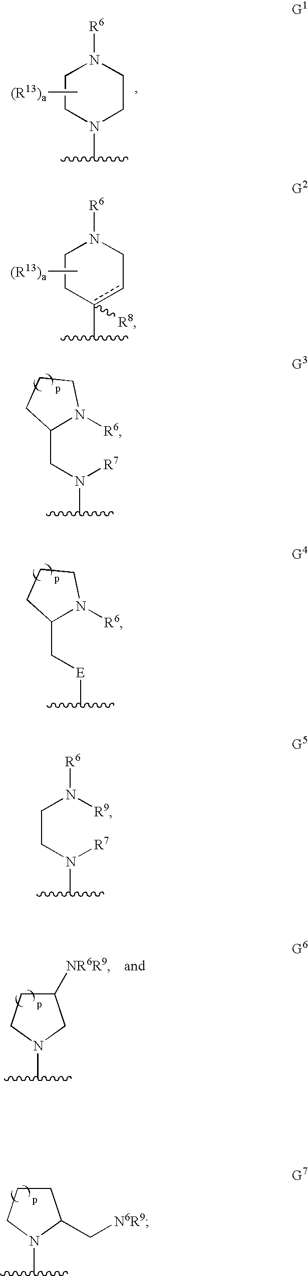
a is an integer from zero to eight;
each R¹³ is, independently, C₁₋₄-alkyl or a C₁₋₄-methylene bridge from one of the ring carbons of the piperazine or piperidine ring of G¹ or G², respectively, to the same or another ring carbon or a ring nitrogen of the piperazine or piperidine ring of G¹ or G², respectively, having an available bonding site, or to a ring carbon of R⁶ having an available bonding site;
E is oxygen, sulfur, SO or SO₂;
X is hydrogen, chloro, fluoro, bromo, iodo, cyano, C₁₋₆-alkyl, hydroxy, trifluoromethyl, C₁₋₆-alkoxy, —SOₜC₁₋₆-alkyl wherein t is zero one or two, —CO₂R¹⁰ or —CONR¹¹R¹²;
Y is an optionally substituted C₁₋₄-heteroalkyl bridge that, together with the atoms to which it is attached, forms a five to seven membered heterocycle containing two to four heteroatoms selected from the group consisting of 1,3-oxazolidin-4-on-5-yl, 1,3-oxazolidin-2,4-dion-5-yl, 4,5-dihydro-1,2-oxazolidin-3-on-4-yl, 1,3-thiazolidin-4-on-5-yl, 1,3-thiazolidin-2,4-dion-5-yl, 1,3-pyrazolidin-4-on-5-yl, 1,3-imidazolidin-2,4-dion-5-1,2-pyrazolidin-3-on-4-yl, 1,2-thiazolidin-1,1,3-trion-4-yl, 1,2-thiazolidin-3-on-4-yl, tetrahydro-1,2-oxazin-3-on-4-yl, tetrahydro-1,3-oxazin-4-on-5-yl, tetrahydro-1,3-oxazin-2,4-dion-5-yl, morpholin-3-on-2-yl, morpholin-3,5-dion-2-yl, 2,3-dihydro-1,4-oxazin-3-on-2-yl, tetrahydro-1,3-thiazin-4-on-5-yl, tetrahydro-1,3-thiazin-2,4-dion-5-yl, tetrahydro-1,2-thiazin-3-on-4-yl, thiomorpholin-3-on-2-yl, thiomorpholin-3,5-dion-2-yl, 2,3-dihydro-1,4-thiazin-3-on-2-yl, hexahydro-1,2-diazin-3-on-4-yl, 4,5-dihydro-2H-pyridazin-3-on-4-yl, hexahydro-1,3-diazin-4-on-5-yl, hexahydro-1,3-diazin-2,4-dion-5-yl, piperazin-2-on-3-yl, piperazin-2,6-dion-3-yl, tetrahydro-1,3,4-thiadiazin-5-on-6-yl, 5,6-dihydro-1,3,4-thiadiazin-5-on-6-yl, 1,3,4-oxadiazin-5-on-6-yl, 5,6-dihydro-1,2,4-oxadiazin-5-on-6-yl, tetrahydro-1,2,4-oxadiazin-5-on-6-yl, 1,2,4-triazin-5-on-6-yl, tetrahydro-1,2,4-oxadiazin-5-on-6-yl, 5,6-dihydro-1,2,4-oxadiazin-5-on-6-yl, 1,2,4-oxadiazin-3,5-dion-6-yl, 1,2,4-trazin-6-on-5-yl, hexahydro-1,2-oxazepin-3-on-2-yl, hexahydro-1,3-oxazepin4-on-5-yl, hexahydro-1,4-oxazepin-3-on-2-yl, hexahydro-1,4-oxazepin-3,5-dion-2-yl, hexahydro-1,4-oxazepin-3,5-dion-6-yl, 2,3,5,6-tetrahydro-1-4-oxazepin-5,7-dion-4-yl, hexahydro-1,4-oxazepin-5-on-6-yl, hexahydro-1,3-oxazepin-2,4-dion-5-yl, hexahydro-1,2-thiazepin-3-on-4-yl, hexahydro-1,4-thiazepin-3-on-2-yl, 2,3,4,5-tetrahydro-1,4-thiazepin-3-on-2-yl, hexahydro-1,4-thiazepin-3,5-dion-2-yl, hexahydro-1,4-thiazepin-3,5-dion-6-yl, 2,3,6,7-tetrahydro-1,4-thiazepin-5-on-6-yl, 6,7-dihydro-1,4-thiazepin-5-on-6-yl, hexahydro-1,3-thiazepin-2,4-dion-5-yl, hexahydro-1,2-diazepin-3-on-4-yl, hexahydro-1,3-diazepin-2,4-dion-5-yl, hexahydro-1,4diazepin-2-on-3-yl, hexahydro-1,4-diazepin-5-on-6-yl, hexahydro-1,4diazepin-5,7-dion-6-yl, hexahydro-1,3,5-thiadiazepin-3-on-7-yl, 4,5,6,7-tetrahydro-1,3,5-thiadiazepin-6-on-7-yl, and 2,3,5,6-tetrahydro-1,2,4-triazepin-3,5-dion-7-yl;
wherein the substituents on any of the carbon atoms capable of supporting an additional bond, of said C₁₋₄-heteroalkyl bridge, are chloro, fluoro, C₁₋₆-alkyl, C₁₋₆-alkoxy, trifluoromethyl or cyano; wherein the substituents on any of the nitrogen atoms capable of supporting an additional bond, of said C₁₋₄-heteroalkyl bridge, are C₁₋₆-alkyl or trifluoromethyl;
R is hydrogen, C₁₋₄-alkyl, phenyl or naphthyl, wherein said phenyl or naphthyl may optionally be substituted with one or more substituents independently selected from chloro, fluoro, bromo, iodo, C₁₋₆-alkyl, C₁₋₆-alkoxy, trifluoromethyl, cyano and —SOₖC₁₋₆-alkyl wherein k is zero, one or two;
R³ is —(CH₂)ₘB, wherein m is zero, one, two or three and B is hydrogen, phenyl, naphthyl or a 5 or 6 membered heteroaryl group containing from one to four heteroatoms in the ring, and wherein each of the foregoing phenyl, naphthyl and heteroaryl groups may optionally be substituted with one or more substituents independently selected from chloro, fluoro, bromo, iodo, C₁₋₆-alkyl, C₁₋₆-alkoxy, C₁₋₆-alkoxyC₁₋₆-alkyl, trifluoromethyl, trifluoromethoxy, cyano, hydroxy, —COOH and —SO,C₁₋₆-alkyl wherein n is zero, one or two;
R⁶ is selected from the group consisting of hydrogen, C₁₋₆-alkyl optionally substituted with C₁₋₆-alkoxy or one to three fluorine atoms, or (C₁₋₄-alkyl)aryl wherein the aryl moiety is phenyl, naphthyl, or heteroaryl-(CH₂)_{q}—, wherein the heteroaryl moiety is selected from the group consisting of pyridyl, pyrimidyl, benzoxazolyl, benzothiazolyl, benzisoxazolyl and benzisothiazolyl and q is zero, one, two, three or four, and wherein said aryl and heteroaryl moieties may optionally be substituted with one or more substituents independently selected from the group consisting of chloro, fluoro, bromo, iodo, C₁₋₆-alkyl, C₁₋₆-alkoxy, trifluoromethyl, cyano and —SO_{g}C₁₋₆-alkyl, wherein g is zero, one or two;
R⁷ is selected from the group consisting of hydrogen, C₁₋₆-alkyl, (C₁₋₄-alkyl)aryl wherein the aryl moiety is phenyl, naphthyl, or heteroaryl-(CH₂)ᵣ—, wherein the heteroaryl moiety is selected from the group consisting of pyridyl, pyrimidyl, benzoxazolyl. benzothiazolyl, benzisoxazolyl and benzisothiazolyl and r is zero, one, two, three or four, and wherein said aryl and heteroaryl moieties may optionally be substituted with one or more substituents independently selected from the group consisting of chloro, fluoro, bromo, iodo, C₁₋₆-alkyl, C₁₋₆-alkoxy, trifluoromethyl, —C(O)—C₁₋₆-alkyl, cyano and —SOⱼC₁₋₆-alkyl, wherein j is zero, one or two;
or R⁶ and R⁷ taken together form a 2 to 4 carbon chain;
R⁸ is hydrogen or C₁₋₃-alkyl;
R⁹ is hydrogen or C₁₋₆-alkyl;
or R⁶ and R⁹, together with the nitrogen atom to which they are attached, form a 5- to 7-membered heteroalkyl ring that may contain from zero to four heteroatoms selected from nitrogen, sulfur and oxygen;
p is one, two, or three;
each of R¹⁰, R¹¹ and R¹² is selected, independently, from the radicals set forth in the definition of R²; or R¹¹ and R¹², together with the nitrogen to which they are attached, form a 5- to 7-membered heteroalkyl ring that may contain from zero to four heteroatoms selected from nitrogen, sulfur and oxygen; and
the broken lines indicate optional double bonds, with the proviso that when the broken line in G² is a double bond that R⁸ is absent;
or a pharmaceutically acceptable salt, enantiomer, diastereomer, N-oxide, crystalline form, hydrate, solvate, active metabolite or prodrug thereof;
and wherein Formula U is a compound depicted by the formula 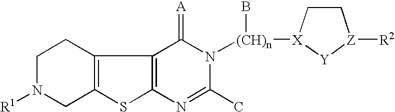
wherein for formula U
R¹ is hydrogen, C₁₋₄-alkyl, acetyl or benzoyl, a phenylalkyl C₁₋₄ radical, wherein the aromatic ring is unsubstituted or substituted by halogen, C₁₋₄-alkyl, trifluoromethyl, hydroxyl, C₁₋₄-alkoxy, amino, cyano or nitro groups, a naphthylalkyl C₁₋₃-radical, a phenylalkanone C₂₋₃-radical or a phenylcarbamoylalkyl C₂ radical, wherein the phenyl ring is unsubstituted or substituted by halogen,
R² is phenyl, pyridyl, pyrimidyl or pyrazinyl, each of which is unsubstituted or carries substituents selected from the group consisting of:
(i) one to three of the following: halogen, C₁₋₄-alkyl, trifluoromethyl, trifluoromethoxy, hydroxyl, C₁₋₄-alkoxy, amino, monomethylamino, dimethylamino, cyano and nitro, and
(ii) one phenyl-C₁₋₂-alkyl or phenyl-C₁₋₂-alkoxy, wherein the phenyl ring is unsubstituted or substituted by halogen, methyl, trilfuoromethyl or methoxy, or
is one of the foregoing unsubstituted or substituted phenyl, pyridyl, pyrimidyl or pyrazinyl radicals wherein two adjacent ring carbon atoms are bridged to form a benzo-fused or a pyridino-fused bicyclic wherein the bridging moiety is unsubstituted or substituted by one or two substituents selected from the group consisting of: halogen, C₁₋₄-alkyl, hydroxyl, trifluoromethyl, C₁₋₄-alkoxy, amino, cyano and nitro, or
is one of the foregoing unsubstituted or substituted phenyl, pyridyl, pyrimidyl or pyrazinyl radicals wherein two adjacent ring carbon atoms are bridged to form a 5- or 6-membered ring consisting of carbon ring members or carbon ring members and one or two oxygen atoms as ring members,
A is NH or an oxygen atom,
B is hydrogen or methyl,
C is hydrogen, methyl or hydroxyl,
X is a nitrogen atom,
Y is CH₂, CH₂—CH₂, CH₂—CH₂—CH₂ or CH₂—CH,
Z is a nitrogen atom, carbon atom or CH, wherein the linkage between Y and Z is a single or a double bond, and
n is 2, 3 or 4,
or a pharmaceutically acceptable salt, enantiomer, diastereomer, N-oxide, crystalline form, hydrate, solvate, active metabolite or prodrug thereof;
and wherein Formula V is a compound depicted by the formula 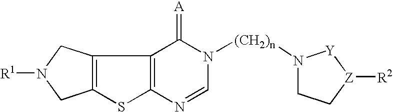
wherein for Formula V
R¹ is a hydrogen atom, a C₁₋₄-alkyl group, an acetyl group, a C₁₋₃-alkyl carboxylate radical, or is a phenyl-C₁₋₄-alkyl radical where the aromatic ring is unsubstituted or substituted by halogen, C₁₋₄-alkyl, trifluoromethyl, hydroxyl, C₁₋₄-alkoxy, amino, cyano or nitro groups,
R² is a phenyl, pyridyl, pyrimidinyl or pyrazinyl group which is unsubstituted or mono- or disubstituted by halogen atoms, C₁₋₄-alkyl, trifluoromethyl, trifluoromethoxy, hydroxyl, C₁₋₄-alkoxy, amino, monomethylamino, dimethylamino, cyano or nitro groups, and may be fused to a benzene nucleus which may be mono- or disubstituted by halogen atoms, C₁₋₄-alkyl, hydroxyl, trifluoromethyl, C₁₋₄-alkoxy, amino, cyano or nitro groups and may contain 1 nitrogen atom, or to a 5- or 6-membered ring which may contain 1-2 oxygen atoms,
A is NH or an oxygen atom,
Y is CH₂, CH₂—CH₂, CH₂—CH₂—CH₂ or CH₂—CH,
Z is a nitrogen atom, carbon atom or CH, where the linkage between Y and Z may also be a double bond, and
n is 2, 3 or 4,
or a pharmaceutically acceptable salt, enantiomer, diastereomer, N-oxide, crystalline form, hydrate, solvate, active metabolite or prodrug thereof,
and wherein Formula W is a compound depicted by the formula 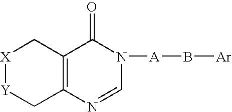
wherein for Formula W
one of the two radicals X and Y is CH₂ and the other is NR¹,
R¹ is hydrogen, C₁₋₈-alkyl, CO—C₁₋₄-alkyl, CO₂tBu, CO-aryl or phenylalkyl C₁₋₄-radical which in turn may be substituted on the aromatic system by F, Cl, Br, I, C₁₋₄-alkyl, C₁₋₄-alkoxy, trifluoromethyl, hydroxyl, amino, cyano or nitro,
A is C₁₋₁₀-alkylene or C₂₋₁₀-alkylene which comprises at least one group Z which is selected from O, S, NR², cyclopropyl, CO₂, CHOH, or a double or triple bond, R² is hydrogen and C₁₋₄-alkyl,
B is 1,4-piperidinylene, 1,2,3,6-tetrahydro-1,4-pyridinylene, 1,4-piperazinylene or the corresponding cyclic compounds enlarged by one methylene group, with the linkage to A being via an N atom of B, and
Ar is phenyl which is unsubstituted or substituted by C₁₋₆-alkyl, O—C₁₋₈-alkyl, F, Cl, Br, I, trifluoromethyl, NR², CO₂R², cyano or phenyl, or is tetratinyl, indanyl, fused aromatic systems which is unsubstituted or substituted by C₁₋₄-alkyl or O—C₁₋₄-alkyl, anthracene or 5- or 6-membered aromatic heterocycles having 1 or 2 heteroatoms which are selected, independently of one another, from O and N, which may be fused to other aromatic radicals,
or a pharmaceutically acceptable salt, enantiomer, diastereomer, N-oxide, crystalline form, hydrate, solvate, active metabolite or prodrug thereof.

5. The method according to claim 1, wherein said compound that has 5HT_{1A} antagonist activity is N-[2-[4-(2-methoxyphenyl)piperazin-1-yl]ethyl]-N-(2-pyridyl)cyclohexanecarboxamide.

6. The method according to claim 1, wherein said compound that has 5HT_{1A} antagonist activity is 2-[4-[4-(7-chloro-2,3-dihydro-1,4-benzodioxin-5-yl)piperazin-1-yl]butyl]-1,2-benzisothiazol-3-(2H)]-one-1,1dioxide.

7. The method according to claim 1, wherein said compound that has 5HT_{1B} antagonist activity is N-[3-[2-(dimethylamino)ethoxy]-4-methoxyphenyl-2′-methyl-4′-(5-methyl-1,2,4-oxadiazol-3-yl)-[1,1′-biphenyl]-4-carboxamide.

8. The method according to claim 1, wherein said compound that has 5HT_{1B} antagonist activity is 1′-methyl-5-[(2′-methyl-4′-(5-methyl-1,2,4-oxadiazo]-3-yl) biphenyl-4-yl]carbonyl]-2,3,6,7-tetrahydrospiro[furo[2,3-f]indole-3,4′-piperidine.

9. The method according to claim 1, wherein said compound that has 5HT_{1A} antagonist activity is 1-[N-(2-nitrophenyl)-N-cyclohexylcarbonyl-2-aminoethyl]-4-(2-methoxyphenyl)piperazine.

10. The method according to claim 1, wherein said compound that has 5HT_{1A} antagonist activity is 1-[3-hydroxy-3,3 bis-(2-pyridyl)propyl]-4-(4-indolyl)piperazine.

11. The method according to claim 3, wherein said compound that has 5HT_{1B} antagonist activity is 3-[3-(dimethylamino)propyl]-4-hydroxy-N-[4-(4-pyridinyl)phenyl]benzamide dihydrochloride.

12. The method according to claim 1, wherein said compound that has 5HT_{1A} antagonist activity is 1-cyclohexyl-4-[4-(2-methoxyphenyl)piperazine-1-yl]-2-(2-pyridyl)butan-1-one.

13. The method according to claim 1, wherein said compound that has both 5HT_{1A} and 5HT_{1B} antagonist activity is (Z)-4-(3,4-dichlorophenyl)-2-[2-(4-methylpiperazin-1-yl)benzylidene]thiomorpholin-3-one.

14. The method according to claim 1 wherein said neuromuscular dysfunction is chosen from the group consisting of urinary urgency, overactive bladder, increased urinary frequency, decreased urinary compliance, cystitis, incontinence, urine leakage, enuresis, dysuria, urinary hesitancy and difficulty in emptying the bladder.

15. The method according to claim 14 wherein said neuromuscular dysfunction is decreased urinary complaince.

16. The method according to claim 14 wherein said neuromuscular dysfunction is cystitis.

17. The method according to claim 1 wherein said compounds are administered in a pharmaceutically acceptable composition.

18. The method according to claim 1 wherein said pharmaceutically acceptable composition is administered via an oral parentemal, intranasal, sublingual, rectal, insufflation, inhalatory route, trasndermal patches or lyophilized composition.

19. The method according to claim 1, wherein each of said compounds are administered in an amount of between about 0.01 to about 25 mg/kg/day.

20. The method according to claim 1 wherein said mammal is human.
